(19) United States
(12) Patent Application Publication
(10) Pub. No.: US 2026/0015351 A1
(43) Pub. Date: Jan. 15, 2026

(54) METHODS AND COMPOUNDS FOR INHIBITING MKK7 ENZYMES

(71) Applicant: AXONIS THERAPEUTICS, INC., Boston, MA (US)

(72) Inventors: Joanna STANICKA, Canton, MA (US); Amanda ZIMMERMAN, Brookline, MA (US); Michael ROWLEY, Chelmsford (GB); Matthew Alexander James DUNCTON, San Bruno, CA (US); Shane HEGARTY, canton, MA (US); Neil Derek MILLER, London (GB)

(21) Appl. No.: 18/880,127

(22) PCT Filed: Jun. 28, 2023
(86) PCT No.: PCT/US2023/026480

§ 371 Date: Dec. 30, 2024

Related U.S. Application Data
(60) Provisional application No. 63/357,190, filed on Jun. 30, 2022.

(51) Int. Cl. C07D 471/04; A61K 31/416; A61K 31/437; A61K 31/4709; A61K 31/538; C07D 401/04; C07D 403/04; C07D 413/04

(52) U.S. Cl. CPC C07D 471/04; A61K 31/416; A61K 31/437; A61K 31/4709; A61K 31/538; C07D 401/04; C07D 403/04; C07D 413/04

(57) ABSTRACT

The disclosure provides compounds of Formula (I) and pharmaceutically acceptable salts thereof. Also provided are compositions and methods of using the compounds, e.g., for the inhibition of MKK7 enzymes, in which inhibition results in treatment of disorders associated with MKK7-related pathway regulation.

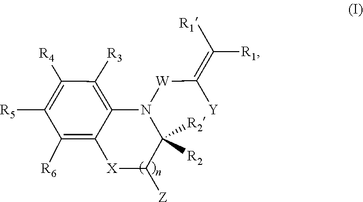

### BACKGROUND

Neurological, inflammatory and cancer disorders are detrimental to individuals due to the impact on a number of essential functions such as neurological (e.g., sensory, motor, cognitive) and/or immune, metabolic, homeostatic, and other organ functions in the affected individual. These disorders often result in profound and irreversible neurological, inflammatory, and cancer-related effects that pose severe challenges to an afflicted patient's everyday life. Few therapeutics have been studied or utilized to treat these disorders, which cause severe challenges and suffering for these patients. Additionally, the few that have been studied or utilized are not adequately sufficient to reduce the individual's suffering or improve recovery from these neurological, inflammatory, and cancer disorders.

Accordingly, there is a need for novel therapeutic agents for the treatment of neurological, inflammatory, and cancer disorders.

### SUMMARY OF THE DISCLOSURE

The present disclosure provides compounds, compositions, and methods for inhibiting MKK7 enzymes in a patient suffering from a disorder associated with MKK7-related pathways and cellular processes, including but not limited to MAPK and JNK signaling pathways and cellular stress responses.

The disclosed methods include administration to a subject suffering from a neurological, inflammatory or cancer disorder of a compound disclosed herein in an amount effective. The disclosure further provides pharmaceutical compositions containing one of the compounds described herein.

In a first aspect, the disclosure provides a compound of formula (I):

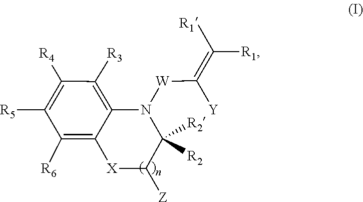

or a pharmaceutically acceptable salt thereof, in which
W is C(O) or S(O)₂, wherein one of R₁ or R₁′ is H, and the other R₁ or R₁′ is H; cyano; optionally substituted C₁-C₄ alkyl; CH₂N(Rₐ)₂, in which each Rₐ is independently H or optionally substituted C₁-C₄ alkyl; or C(O)ORₐ′, in which Rₐ′ is optionally substituted C₁-C₄ alkyl or H,
Y is halo or H;
each of R₂ and R₂′ is independently H, optionally substituted C₁-C₄ alkyl, or optionally substituted C₃-C₆ cycloalkyl;
Z, if present, is C₁-C₄ alkyl;
n is 0 or 1;
X is C(R_{b})₂; O; or NR_{b}′;
each of R_{b} and R_{b}′ is independently H or optionally substituted C₁-C₄ alkyl;
each of R₃ and R₅ is independently H, halo, optionally substituted C₁-C₄ alkyl, optionally substituted C₁-C₄ alkoxy, optionally substituted C₁-C₄ haloalkyl, optionally substituted C₁-C₄ haloalkoxy, or cyano;
one of R₄ and R₆ is H, halo, optionally substituted C₁-C₄ alkyl, optionally substituted C₁-C₄ alkoxy, optionally substituted C₁-C₄ haloalkyl, optionally substituted C₁-C₄ haloalkoxy, or cyano; and the remaining R₄ or R₆ is

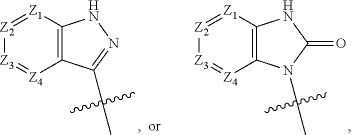

in which
each of Z₁, Z₂, Z₃, and Z₄ is independently CR_{c} or N, in which R_{c} is H, halo, optionally substituted C₁-C₄ alkyl, optionally substituted C₁-C₄ alkoxy, optionally substituted C₁-C₄ haloalkyl, optionally substituted C₁-C₄ haloalkoxy, or cyano.

In some embodiments, R₄ or R₆ is

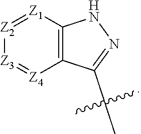

In some embodiments, the compound is a compound of formula (I-2):

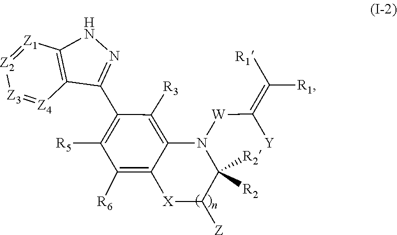

or a pharmaceutically acceptable salt thereof.

In some embodiments, the compound is a compound of formula (I-3):

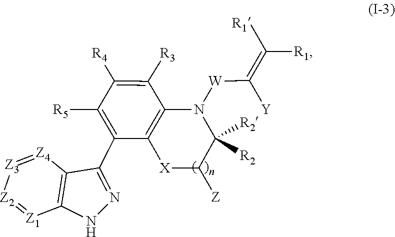

or a pharmaceutically acceptable salt thereof.

In some embodiments, R₄ or R₆ is

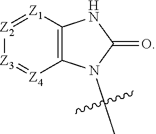

In some embodiments, the compound is a compound of formula (I-4):

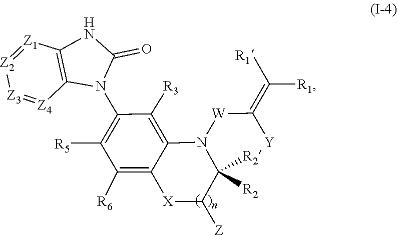

or a pharmaceutically acceptable salt thereof.

In some embodiments, the compound is a compound of formula (I-5):

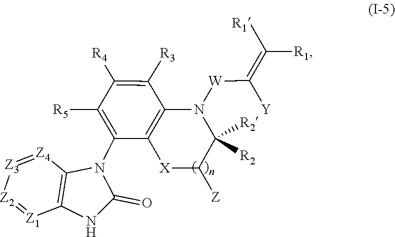

or a pharmaceutically acceptable salt thereof.

In some embodiments of any of the aspects described herein (e.g., formula (I), (I-2), or (I-4)), R₆ is H.

In some embodiments of any of the aspects described herein (e.g., formula (I), (I-3), or (I-5)), R₄ is H.

In some embodiments of any of the aspects described herein (e.g., formula (I), (I-2), (I-3), (I-4), or (I-5)), Z₁ is CR_{c}, in which R_{c} is H, cyano, or optionally substituted C₁-C₄ alkyl, e.g., CH or CCH₃.

In some embodiments of any of the aspects described herein (e.g., formula (I), (I-2), (I-3), (I-4), or (I-5)), Z₁ is N. In some embodiments of any of the aspects described herein (e.g., formula (I), (I-2), (I-3), (I-4), or (I-5)), Z₁ is N, W is S(O)₂, and R₁, R₁′ and Y are each H. In some embodiments of any of the aspects described herein (e.g., formula (I), (I-2), (I-3), (I-4), or (I-5)), Z₁ is N, W is C(O), R₁, R₁′ and Y are each H, and R₂ is C₁-C₄ alkyl, e.g., methyl. In some embodiments of any of the aspects described herein (e.g., formula (I), (I-2), (I-3), (I-4), or (I-5)), Z₁ is N, W is C(O), R₁, R₁′ and Y are each H, and R₂′ is C₁-C₄ alkyl, e.g., methyl. In some embodiments of any of the aspects described herein (e.g., formula (I), (I-2), (I-3), (I-4), or (I-5)), Z₁ is N, W is C(O), R₁, R₁′ and Y are each H, and R₃ is halo, e.g., fluoro.

In some embodiments of any of the aspects described herein (e.g., formula (I), (I-2), (I-3), (I-4), or (I-5)), Z₂ is CR_{c}, in which R_{c} is H, cyano, or optionally substituted C₁-C₄ alkyl, e.g., CH or CCH₃.

In some embodiments of any of the aspects described herein (e.g., formula (I), (I-2), (I-3), (I-4), or (I-5)), Z₂ is N.

In some embodiments of any of the aspects described herein (e.g., formula (I), (I-2), (I-3), (I-4), or (I-5)), Z₃ is CR_{c}, in which R_{c} is H, cyano, or optionally substituted C₁-C₄ alkyl, e.g., CH or CCH₃.

In some embodiments of any of the aspects described herein (e.g., formula (I), (I-2), (I-3), (I-4), or (I-5)), Z₃ is N.

In some embodiments of any of the aspects described herein (e.g., formula (I), (I-2), (I-3), (I-4), or (I-5)), Z₄ is CR_{c}, in which R_{c} is H, cyano, or optionally substituted C₁-C₄ alkyl, e.g., CH or CCH₃.

In some embodiments of any of the aspects described herein (e.g., formula (I), (I-2), (I-3), (I-4), or (I-5)), Z₄ is N.

In some embodiments of any of the aspects described herein (e.g., formula (I), (I-2), (I-3), (I-4), or (I-5)), W is C(O).

In some embodiments of any of the aspects described herein (e.g., formula (I), (I-2), (I-3), (I-4), or (I-5)), W is S(O)₂.

In some embodiments of any of the aspects described herein (e.g., formula (I), (I-2), (I-3), (I-4), or (I-5)), R₁ is cyano.

In some embodiments of any of the aspects described herein (e.g., formula (I), (I-2), (I-3), (I-4), or (I-5)), R₁ is CH₂N(Rₐ)₂, in which each Rₐ is optionally substituted C₁-C₄ alkyl, e.g., methyl.

In some embodiments of any of the aspects described herein (e.g., formula (I), (I-2), (I-3), (I-4), or (I-5)), R₁ is C(O)ORₐ′, in which Rₐ′ is optionally substituted C₁-C₄ alkyl, e.g., ethyl.

In some embodiments of any of the aspects described herein (e.g., formula (I), (I-2), (I-3), (I-4), or (I-5)), R₁ is optionally substituted C₁-C₄ alkyl, e.g., methyl.

In some embodiments of any of the aspects described herein (e.g., formula (I), (I-2), (I-3), (I-4), or (I-5)), R₁′ is cyano.

In some embodiments of any of the aspects described herein (e.g., formula (I), (I-2), (I-3), (I-4), or (I-5)), R₁′ is CH₂N(Rₐ)₂, in which each Rₐ is optionally substituted C₁-C₄ alkyl, e.g., methyl.

In some embodiments of any of the aspects described herein (e.g., formula (I), (I-2), (I-3), (I-4), or (I-5)), R₁′ is C(O)ORₐ′, in which Rₐ′ is optionally substituted C₁-C₄ alkyl, e.g., ethyl.

In some embodiments of any of the aspects described herein (e.g., formula (I), (I-2), (I-3), (I-4), or (I-5)), R₁′ is optionally substituted C₁-C₄ alkyl, e.g., methyl.

In some embodiments of any of the aspects described herein (e.g., formula (I), (I-2), (I-3), (I-4), or (I-5)), Y is halo, e.g., fluoro.

In some embodiments of any of the aspects described herein (e.g., formula (I), (I-2), (I-3), (I-4), or (I-5)), Y is H.

In some embodiments of any of the aspects described herein (e.g., formula (I), (I-2), (I-3), (I-4), or (I-5)), R₂ is H.

In some embodiments of any of the aspects described herein (e.g., formula (I), (I-2), (I-3), (I-4), or (I-5)), R₂ is optionally substituted C₁-C₄ alkyl, e.g., methyl.

In some embodiments of any of the aspects described herein (e.g., formula (I), (I-2), (I-3), (I-4), or (I-5)), R₂ is optionally substituted C₃-C₆ cycloalkyl.

In some embodiments of any of the aspects described herein (e.g., formula (I), (I-2), (I-3), (I-4), or (I-5)), R₂′ is H. In some embodiments of any of the aspects described herein (e.g., formula (I), (I-2), (I-3), (I-4), or (I-5)), R₂′ is optionally substituted C₁-C₄ alkyl, e.g., methyl.

In some embodiments of any of the aspects described herein (e.g., formula (I), (I-2), (I-3), (I-4), or (I-5)), R₂′ is optionally substituted C₃-C₆ cycloalkyl.

In some embodiments of any of the aspects described herein (e.g., formula (I), (I-2), (I-3), (I-4), or (I-5)), n is 1.

In some embodiments of any of the aspects described herein (e.g., formula (I), (I-2), (I-3), (I-4), or (I-5)), n is 0.

In some embodiments of any of the aspects described herein (e.g., formula (I), (I-2), (I-3), (I-4), or (I-5)), X is C(R_{b})₂, in which each R_{b} is independently H.

In some embodiments of any of the aspects described herein (e.g., formula (I), (I-2), (I-3), (I-4), or (I-5)), X is C(R_{b})₂, in which each R_{b} is independently optionally substituted C₁-C₄ alkyl.

In some embodiments of any of the aspects described herein (e.g., formula (I), (I-2), (I-3), (I-4), or (I-5)), X is O.

In some embodiments of any of the aspects described herein (e.g., formula (I), (I-2), (I-3), (I-4), or (I-5)), X is NR_{b}′, in which R_{b}′ is H.

In some embodiments of any of the aspects described herein (e.g., formula (I), (I-2), (I-3), (I-4), or (I-5)), X is NR_{b}′, in which R_{b}′ is optionally substituted C₁-C₄ alkyl.

In some embodiments of any of the aspects described herein (e.g., formula (I), (I-2), (I-3), (I-4), or (I-5)), R₃ is H.

In some embodiments of any of the aspects described herein (e.g., formula (I), (I-2), (I-3), (I-4), or (I-5)), R₅ is H.

In another aspect, the present disclosure provides compounds of Table 1:

TABLE 1

| Compound | Structure |
|---|---|
| 1 | 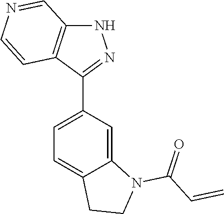 [SMILES: C=CC(=O)N1CCc2ccc(-c3n[nH]c4cnccc34)cc21] |
| 2 | 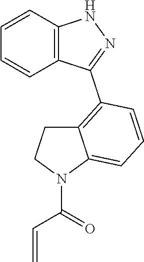 [SMILES: C=CC(=O)N1CCc2c(-c3n[nH]c4ccccc34)cccc21] |
| 3 | 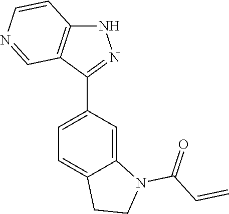 [SMILES: C=CC(=O)N1CCc2ccc(-c3n[nH]c4ccncc34)cc21] |
| 4 | 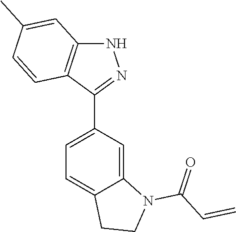 [SMILES: C=CC(=O)N1CCc2ccc(-c3n[nH]c4cc(C)ccc34)cc21] |
| 5 | 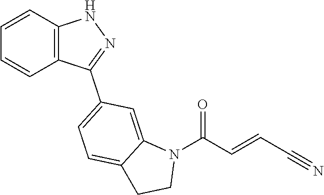 [SMILES: N#C/C=C/C(=O)N1CCc2ccc(-c3n[nH]c4ccccc34)cc21] |
| 6 | 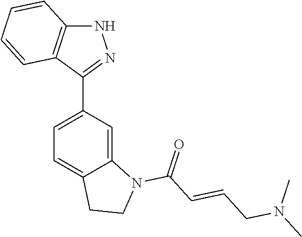 [SMILES: CN(C)C/C=C/C(=O)N1CCc2ccc(-c3n[nH]c4ccccc34)cc21] |
| 7 | 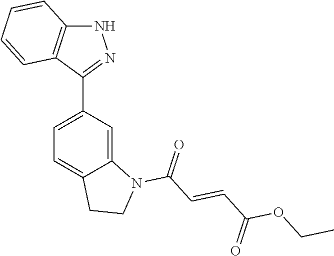 [SMILES: CCOC(=O)/C=C/C(=O)N1CCc2ccc(-c3n[nH]c4ccccc34)cc21] |
| 8 | 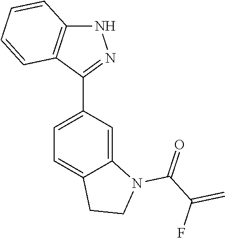 [SMILES: C=C(F)C(=O)N1CCc2ccc(-c3n[nH]c4ccccc34)cc21] |
| 9 | 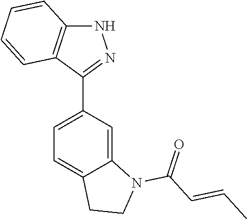 [SMILES: C/C=C/C(=O)N1CCc2ccc(-c3n[nH]c4ccccc34)cc21] |
| 10 | 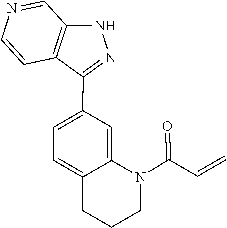 [SMILES: C=CC(=O)N1CCCc2ccc(-c3n[nH]c4cnccc34)cc21] |
| 11 | 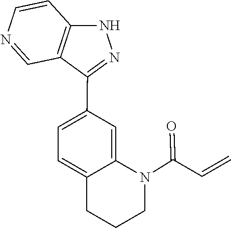 [SMILES: C=CC(=O)N1CCCc2ccc(-c3n[nH]c4ccncc34)cc21] |
| 12 | 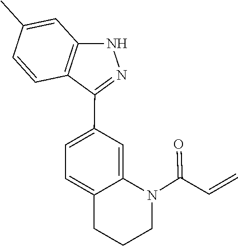 [SMILES: C=CC(=O)N1CCCc2ccc(-c3n[nH]c4cc(C)ccc34)cc21] |
| 13 | 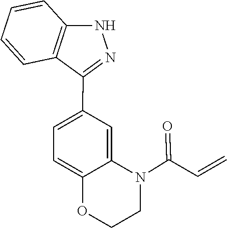 [SMILES: C=CC(=O)N1CCOc2ccc(-c3n[nH]c4ccccc34)cc21] |
| 14 | 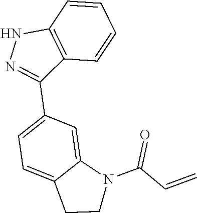 [SMILES: C=CC(=O)N1CCc2ccc(-c3n[nH]c4ccccc34)cc21] |
| 15 | 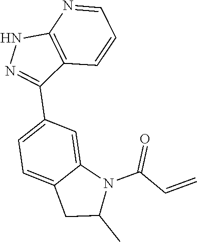 [SMILES: C=CC(=O)N1c2cc(-c3n[nH]c4ncccc34)ccc2CC1C] |
| 15-A | 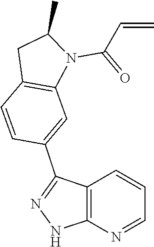 [SMILES: C=CC(=O)N1c2cc(-c3n[nH]c4ncccc34)ccc2C[C@H]1C] |
| 15-B | 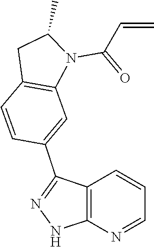 [SMILES: C=CC(=O)N1c2cc(-c3n[nH]c4ncccc34)ccc2C[C@@H]1C] |
| 16 | 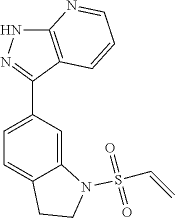 [SMILES: C=CS(=O)(=O)N1CCc2ccc(-c3n[nH]c4ncccc34)cc21] |
| 17 | 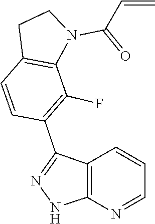 [SMILES: C=CC(=O)N1CCc2ccc(-c3n[nH]c4ncccc34)c(F)c21] |
| 18 | 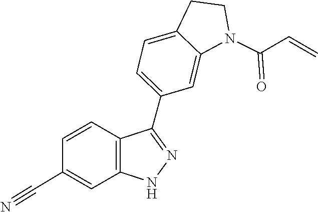 [SMILES: C=CC(=O)N1CCc2ccc(-c3n[nH]c4cc(C#N)ccc34)cc21] |

In another aspect, the disclosure provides a pharmaceutically composition including a compound described herein (e.g., any one of the compounds of Formulas (I), (I-2), (I-3), (I-4), (I-5), and Table 1) or a pharmaceutically acceptable salt thereof and a pharmaceutically acceptable excipient.

In another aspect, the disclosure provides a method for inhibiting an MKK7 enzyme, which includes administering to a subject in need thereof a therapeutically effective amount of a compound described herein (e.g., any one of the compounds of Formulas (I), (I-2), (I-3), (I-4), (I-5), and Table 1) or a pharmaceutically acceptable salt thereof.

In another aspect, the disclosure provides a method for increasing survival, or reducing death or degeneration of a damaged or degenerating neuron, which includes contacting the damaged or degenerating neuron by administering to a subject in need thereof a therapeutically effective amount of a compound described herein (e.g., any one of the compounds of Formulas (I), (I-2), (I-3), (I-4), (I-5), and Table 1) or a pharmaceutically acceptable salt thereof.

In another aspect, the disclosure provides a method for treating an inflammatory disorder or cancer by administering to a subject in need thereof a therapeutically effective amount of a compound described herein (e.g., any one of the compounds of Formulas (I), (I-2), (I-3), (I-4), (I-5), and Table 1) or a pharmaceutically acceptable salt thereof.

In some embodiments, the inflammatory disorder is intraocular inflammation. In some embodiments, the cancer is neuroblastoma or glioblastoma.

In another aspect, the disclosure provides a method for treating a disorder associated with MKK7-related pathway regulation, which includes administering to a subject in need thereof a therapeutically effective amount of a compound described herein (e.g., any one of the compounds of Formulas (I), (I-2), (I-3), (I-4), (I-5), and Table 1) or a pharmaceutically acceptable salt thereof.

In some embodiments, the disorder is associated with an MKK7 enzyme.

In some embodiments, the disorder is a neurodegenerative disease, a neurotraumatic disorder, a neurodevelopmental disorder, or an affective disorder.

In some embodiments, the disorder is amyotrophic lateral sclerosis (ALS), glaucoma, chemotherapy-induced peripheral neuropathy, Alzheimer's disease, Parkinson's disease, Huntington's disease, multiple sclerosis (MS), optic neuropathy, neuroblastoma, glioblastoma, lysosomal storage disorders, traumatic brain injury, spinal cord injury, spinal cord crush, optic nerve injury, or a combination thereof.

### Definitions

To facilitate the understanding of the present disclosure, a number of terms are defined below. Terms defined herein have meanings as commonly understood by a person of ordinary skill in the areas relevant to the disclosure. Terms such as “a,” “an,” and “the” are not intended to refer to only a singular entity, but include the general class of which a specific example may be used for illustration. The terminology herein is used to describe specific embodiments of the disclosure, but their usage does not limit the disclosure, except as outlined in the claims.

As used herein, the term “about” is used to indicate that a value includes the standard deviation of error for the method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used herein, any values provided in a range of values include both the upper and lower bounds, and any values contained within the upper and lower bounds.

As used herein, the terms “administer” and “administering” are used to indicate the process of providing a therapeutic, pharmaceutical, housing compartment, medication, or the like thereof to a subject. In some embodiments, a pharmaceutical is provided via oral administration.

As used herein, the terms “improve” and “improving,” in reference to recovery from a disease or condition, e.g., a disorder, refer to an enhancement of recovery in one or more parameters measuring or quantifying the severity of the disorder relative to the recovery in these parameters in or prior to treatment with the compounds or compositions described herein. Alternatively, improvement may be measured with respect to a reference subject having the same diagnosis as the subject but that did not receive treatment with a compound or composition of the disclosure. For disorders, such parameters may include motor and sensory function in a subject. Methods for assessing motor and sensory function in a subject suffering from a disorder are known in the art and are further described herein.

As used herein, the term “degenerating” or “degeneration,” refers to a biological state of becoming degenerate or declining. A neuron or cell may be degenerating or declining when there is a reduced likelihood of cell viability, which may be measured by one or more cell viability measurements, e.g., metabolic assays (e.g., MTT assay, XTT assay, etc.), ATP measurement assays, etc. Degeneration may be determined by determining a number of cells undergoing apoptosis via an apoptosis assay, annexin V assay, caspase assay, chromatin condensation assay, TUNEL assay, cytochrome c release assay, etc.

As used herein, the term “pharmaceutical composition” refers to an active compound, formulated together with one or more pharmaceutically acceptable excipients. In some embodiments, a compound of the disclosure is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions) or tablets, e.g., those targeted for buccal, sublingual, and systemic absorption, boluses, powders, granules, or pastes for application to the tongue.

The term “pharmaceutically acceptable excipient,” as used herein, refers to any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes, emollients, emulsifiers, diluents, film formers or coatings, flavors, fragrances, glidants, lubricants, preservatives, printing inks, sorbents, suspending or dispersing agents, sweeteners, or waters of hydration. Excipients include, but are not limited to: butylated optionally substituted hydroxytoluene (e.g., BHT), calcium carbonate, calcium phosphate dibasic, calcium stearate, croscarmellose, crosslinked polyvinyl pyrrolidone, citric acid, crospovidone, cysteine, ethylcellulose, gelatin, optionally substituted hydroxypropyl cellulose, optionally substituted hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch, stearic acid, stearic acid, sucrose, talc, titanium dioxide, vitamin A, vitamin E, vitamin C, and xylitol. Those of ordinary skill in the art are familiar with a variety of agents and materials useful as excipients.

As used herein, the term “pharmaceutically acceptable salt” represents those salts of the compounds described that are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in Handbook of Pharmaceutical Salts: Properties, Selection, and Use, (Eds. P. H. Stahl and C. G. Wermuth), Wiley-VCH, 2008. These salts may be acid addition salts involving inorganic or organic acids. The salts can be prepared in situ during the final isolation and purification of the compounds described herein or separately by reacting the free base group with a suitable acid. Methods for preparation of the appropriate salts are well-established in the art.

The term “subject,” as used herein, can be a human, non-human primate, or other mammal, such as but not limited to dog, cat, horse, cow, pig, goat, monkey, rat, mouse, and sheep. In preferred embodiments, the subject is a human.

As used herein, the term “survival,” refers to a biological state of continuing to exist or remain alive. A neuron or cell may have an increased survival comprising an increased likelihood of cell viability, which may be measured by one or more cell viability measurements, e.g., metabolic assays (e.g., MTT assay, XTT assay, etc.), ATP measurement assays, etc. Survival may be determined by determining a number of cells undergoing apoptosis via an apoptosis assay, annexin V assay, caspase assay, chromatin condensation assay, TUNEL assay, cytochrome c release assay, etc.

As used herein, the term “therapeutically effective amount” refers to an amount sufficient to effect beneficial or desired results, such as clinical results, and, as such, a “therapeutically effective amount” depends upon the context in which it is being applied. For example, in the context of administering a compound disclosed herein (e.g., a compound of any one of formulas (I), (I-2), (I-3), (I-4), (I-5), and other compounds disclosed herein) to treat a neurological disorder, a therapeutically effective amount of a compound is, for example, an amount sufficient to reverse alleviate the neurological disorder.

As used herein, the term “treat,” or “treating,” refers to a therapeutic treatment of a disorder in a subject. The effect of treatment can include reversing, alleviating, reducing severity of, inhibiting the progression of, reducing the likelihood of recurrence of the disorder or one or more symptoms or manifestations of the disorder, stabilizing (i.e., not worsening) the state of the disorder as compared to the state and/or the condition of the disease or disorder in the absence of the therapeutic treatment.

The term “alkenyl,” as used herein, refers to a branched or straight-chain monovalent unsaturated aliphatic radical containing at least one carbon-carbon double bond and no carbon-carbon triple bonds, and only C and H when unsubstituted. Monovalency of an alkenyl group does not include the optional substituents on the alkenyl group. For example, if an alkenyl group is attached to a compound, monovalency of the alkenyl group refers to its attachment to the compound and does not include any additional substituents that may be present on the alkenyl group. In some embodiments, the alkenyl group may contain, e.g., 2-20, 2-18, 2-16, 2-14, 2-12, 2-10, 2-8, 2-6, or 2-4 carbon atoms (e.g., C₂-C₂₀, C₂-C₁₈, C₂-C₁₆, C₂-C₁₄, C₂-C₁₂, C₂-C₁₀, C₂-C₈, C₂-C₆, or C₂-C₄). Examples include, but are not limited to, ethenyl, 1-propenyl, 2-propenyl, 1-methylethenyl, 1-butenyl, 2-butenyl, 3-butenyl, and the like.

The term “alkoxy,” as used herein, refers to a monovalent radical of formula —OR, in which R is alkyl.

The term “alkyl,” as used herein, refers to a branched or straight-chain monovalent saturated aliphatic radical containing only C and H when unsubstituted. The monovalency of an alkyl group does not include the optional substituents on the alkyl group. For example, if an alkyl group is attached to a compound, monovalency of the alkyl group refers to its attachment to the compound and does not include any additional substituents that may be present on the alkyl group. In some embodiments, the alkyl group may contain, e.g., 1-20, 1-18, 1-16, 1-14, 1-12, 1-10, 1-8, 1-6, 1-4, or 1-2 carbon atoms (e.g., C₁-C₂₀, C₁-C₁₈, C₁-C₁₆, C₁-C₁₄, C₁-C₁₂, C₁-C₁₀, C₁-C₈, C₁-C₆, C₁-C₄, or C₁-C₂). Examples include, but are not limited to, methyl, ethyl, isobutyl, sec-butyl, and tert-butyl.

The term “alkynyl,” as used herein, refers to a branched or straight-chain monovalent unsaturated aliphatic radical containing at least one carbon-carbon triple bond and only C and H when unsubstituted.

Monovalency of an alkynyl group does not include the optional substituents on the alkynyl group. For example, if an alkynyl group is attached to a compound, monovalency of the alkynyl group refers to its attachment to the compound and does not include any additional substituents that may be present on the alkynyl group. In some embodiments, the alkynyl group may contain, e.g., 2-20, 2-18, 2-16, 2-14, 2-12, 2-10, 2-8, 2-6, or 2-4 carbon atoms (e.g., C₂-C₂₀, C₂-C₁₈, C₂-C₁₆, C₂-C₁₄, C₂-C₁₂, C₂-C₁₀, C₂-C₈, C₂-C₆, or C₂-C₄). Examples include, but are not limited to, ethynyl, 1-propynyl, and 3-butynyl.

The term “aryl,” as used herein, refers to any monocyclic or fused ring bicyclic or multicyclic system containing only carbon atoms in the ring(s), which has the characteristics of aromaticity in terms of electron distribution throughout the ring system, e.g., phenyl, naphthyl, or phenanthryl. An aryl group may have, e.g., six to sixteen carbons or six to fourteen carbons (e.g., six carbons, ten carbons, thirteen carbons, fourteen carbons, or sixteen carbons).

The term “carbocycle,” as used herein, refers to a monovalent, saturated (“cycloalkyl”) or unsaturated, non-aromatic cyclic group containing only C and H when unsubstituted. A carbocycle may have, e.g., three to twenty carbons (e.g., a C₃-C₇, C₃-C₈, C₃-C₉, C₃-C₁₀, C₃-C₁₁, C₃-C₁₂, C₃-C₁₄, C₃-C₁₆, C₃-C₁₈, or C₃-C₂₀ carbocycle).

The term “cyano,” as used herein, refers to a monovalent radical of formula —CN.

The term “cycloalkyl,” as used herein, refers to a monovalent, saturated cyclic group containing only C and H when unsubstituted. A cycloalkyl may have, e.g., three to twenty carbons (e.g., a C₃-C₇, C₃-C₈, C₃-C₉, C₃-C₁₀, C₃-C₁₁, C₃-C₁₂, C₃-C₁₄, C₃-C₁₆, C₃-C₁₈, or C₃-C₂₀ cycloalkyl). Examples of cycloalkyls include, but are not limited to, cyclopropyl, cyclobutyl, cyclopentyl, cyclohexyl, and cycloheptyl.

The term “cycloalkyl” also includes cyclic groups having a bridged multicyclic structure in which one or more carbons bridges two non-adjacent members of a monocyclic ring, e.g., bicyclo[2.2.1]heptyl and adamantyl. The term “cycloalkyl” also includes bicyclic, tricyclic, and tetracyclic fused ring structures, e.g., decalin and spiro-cyclic compounds.

The term “halo” or “halogen,” as used herein, refers to a fluorine (fluoro), chlorine (chloro), bromine (bromo), or iodine (iodo) radical.

The term “haloalkyl,” as used herein, refers to an alkyl group, as defined herein, in which one or more hydrogens are replaced with halogen atoms (i.e., alkyl substituted with one or more halo). An example of a haloalkyl group is CF₃.

The term “haloalkoxy,” as used herein, refers to an alkoxy group, as defined herein, in which one or more hydrogen atoms are replaced with halogen atoms (i.e., alkoxy substituted with one or more halo).

An example of a haloalkoxy group is OCF₃.

The term “heterocycle,” as used herein, represents a monocyclic or fused ring bicyclic or multicyclic system having at least one heteroatom as a ring atom. For example, a heterocycle ring may have, e.g., one to fifteen carbons ring atoms (e.g., a C₁-C₂, C₁-C₃, C₁-C₄, C₁-C₅, C₁-C₆, C₁-C₇, C₁-C₈, C₁-C₉, C₁-C₁₀, C₁-C₁, C₁-C₁₂, C₁-C₁₃, C₁-C₁₄, or C₁-C₁₅ heterocycle) and one or more (e.g., one, two, three, four, or five) ring heteroatoms independently selected from the group consisting of nitrogen, oxygen, and sulfur. Heterocycle groups may or may not include a ring that is aromatic. An aromatic heterocycle group is referred to as a “heteroaryl” group. In preferred embodiments of the disclosure, a heterocycle group is a 3- to 8-membered ring, a 3- to 6-membered ring, a 4- to 6-membered ring, a 6- to 10-membered ring, a 6- to 12-membered ring, a 5-membered ring, or a 6-membered ring. Exemplary 5-membered heterocycle groups may have zero to two double bonds, and exemplary 6-membered heterocycle groups may have zero to three double bonds. Exemplary 5-membered groups include, for example, optionally substituted pyrrole, optionally substituted pyrazole, optionally substituted isoxazole, optionally substituted pyrrolidine, optionally substituted imidazole, optionally substituted thiazole, optionally substituted thiophene, optionally substituted thiolane, optionally substituted furan, optionally substituted tetrahydrofuran, optionally substituted diazole, optionally substituted triazole, optionally substituted tetrazole, optionally substituted oxazole, optionally substituted 1,3,4-oxadiazole, optionally substituted 1,3,4-thiadiazole, optionally substituted 1,2,3,4-oxatriazole, and optionally substituted 1,2,3,4-thiatriazole. Exemplary 6-membered heterocycle groups include, for example, optionally substituted pyridine, optionally substituted piperidine, optionally substituted piperazine, optionally substituted pyrimidine, optionally substituted pyrazine, optionally substituted pyridazine, optionally substituted triazine, optionally substituted 2H-pyran, optionally substituted 4H-pyran, and optionally substituted tetrahydropyran. Exemplary 7-membered heterocycle groups include optionally substituted azepine, optionally substituted 1,4-diazepine, optionally substituted thiepine, and optionally substituted 1,4-thiazepine.

As used herein, the term “disorder” refers to any damage or dysfunction in a subject that is associated with Mitogen-activated protein kinase kinase 7 (MKK7)-related pathway regulation, e.g., c-Jun-NH₂-terminal kinase (JNK) pathway regulation. The JNK pathway is a signaling cassette of the mitogen-activated protein kinase (MAPK) signaling pathway, wherein damage or dysfunction may include reduced proliferation of cells, reduced embryonic development, spontaneous apoptosis, or reduced apoptosis. A disorder may include any damage or dysfunction such as enhanced oncogenic transformation. A disorder may include a neurological disorder. A neurological disorder may include any damage or dysfunction that prevents and/or inhibits one or more electrical and/or chemical transmissions of a sensory and/or motor function signal. A neurological disorder may include any damage or dysfunction that results in a transmission of one or more electrical and/or chemical transmissions of a nerve cell uncontrollably by the subject. A neurological disorder may include damage or dysfunction of one or more nerves located within the central nervous system and/or peripheral nervous system of a subject. A neurological disorder may include damage or dysfunction of a somatic, autonomic, and/or enteric nervous system of a subject. A neurological disorder may include damage or dysfunction of an afferent and/or efferent nervous system of a subject. A neurological disorder may include damage or dysfunction of a sympathetic and/or parasympathetic nervous system of a subject.

The term “oxo,” as used herein, refers to a divalent oxygen atom represented by the structure ═O.

The phrase “optionally substituted X,” as used herein, is intended to be equivalent to “X, in which X is optionally substituted” (e.g., “alkyl, in which said alkyl is optionally substituted”). It is not intended to mean that the feature “X” (e.g. alkyl) per se is optional. The term “optionally substituted,” as used herein, refers to having 0, 1, or more substituents (e.g., 0-25, 0-20, 0-10, or 0-5 substituents).

Alkyl, alkylene, alkenyl, alkynyl, carbocycle, cycloalkyl, aryl, and heterocycle groups may be substituted with carbocycle (e.g., cycloalkyl); aryl; heterocycle; halo; OR^{a}, in which Rₐ is H, alkyl, alkenyl, alkynyl, carbocycle (e.g., cycloalkyl), aryl, or heterocycle; SR^{a}, in which Rₐ is as defined herein; CN; NO₂; N₃; NR^{b}R^{c}, in which each of R^{b} and R^{c} is, independently, H, alkyl, alkenyl, alkynyl, carbocycle (e.g., cycloalkyl), aryl, or heterocycle; SO₂R^{d}, in which R^{d} is H, alkyl or aryl; SO₂NR^{e}R^{f}, in which each of R^{e} and R^{f} is, independently, H, alkyl, or aryl; SOR⁹, in which R₉ is H, alkyl, or aryl; or P(O)(OR^{h})₂, in which each R^{h} is, independently, H or alkyl. Aryl, carbocycle (e.g., cycloalkyl), heteroaryl, and heterocycle groups may also be substituted with alkyl, alkenyl, or alkynyl. Alkyl, alkylene, alkenyl, alkynyl, carbocycle (e.g., cycloalkyl), and heterocycle groups may also be substituted with oxo or =NRJ, in which R₁ is H or alkyl. In some embodiments, a substituent is further substituted as described herein. For example, a C₁ alkyl group, i.e., methyl, may be substituted with oxo to form a formyl group and further substituted with —OH or —NR^{b}R^{c} to form a carboxyl group (—COOH) or an amido group (—C(O)NR^{b}R_{c}).

### DETAILED DESCRIPTION

Described herein are compounds, compositions, and methods for inhibiting MKK7 enzymes. Without wishing to be bound by theory, the compounds described herein, such as Compound 1 may function as orally-available drugs of Mitogen-activated protein kinase kinase 7 (MKK7) inhibitors:

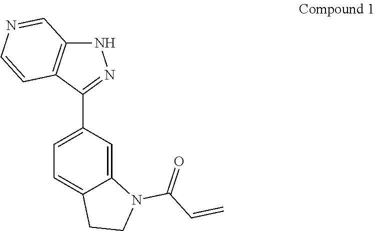 [SMILES: C=CC(=O)N1CCc2ccc(-c3n[nH]c4cnccc34)cc21]

Also described herein are compounds, compositions, and methods for treating disorders associated with MKK7-related pathway regulation, or cellular stress pathways, e.g., neurodegenerative diseases, neurotraumatic disorders, neurodevelopmental disorders, inflammatory, cancer, or affective disorders, by inhibiting one or more MKK7 enzymes.

### Compounds

The present disclosure provides compounds and compositions that can be administered to a subject (e.g., a human) in order to treat a disorder associated with MKK7-related pathway regulation or cellular stress pathways (e.g., a neurodegenerative disease, neurotraumatic disorder, a neurodevelopmental disorder, inflammatory, cancer, or an affective disorder).

In one aspect, the present disclosure provides a compound of Formula (I):

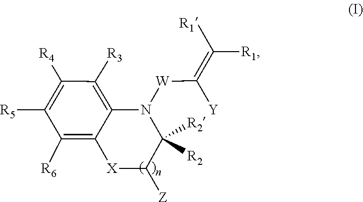

or a pharmaceutically acceptable salt thereof, in which
W is C(O) or S(O)₂, wherein one of R₁ or R₁′ is H, and the other R₁ or R₁′ is H; cyano; optionally substituted C₁-C₄ alkyl; CH₂N(Rₐ)₂, in which each Rₐ is independently H or optionally substituted C₁-C₄ alkyl; or C(O)ORₐ′, in which Rₐ′ is optionally substituted C₁-C₄ alkyl or H,
Y is halo or H;
each of R₂ and R₂′ is independently H, optionally substituted C₁-C₄ alkyl, or optionally substituted C₃-C₆ cycloalkyl;
Z, if present, is C₁-C₄ alkyl;
n is 0 or 1;
X is C(R_{b})₂, O; or NR_{b}′;
each of R_{b} and R_{b}′ is independently H or optionally substituted C₁-C₄ alkyl;
each of R₃ and R₅ is independently H, halo, optionally substituted C₁-C₄ alkyl, optionally substituted C₁-C₄ alkoxy, optionally substituted C₁-C₄ haloalkyl, optionally substituted C₁-C₄ haloalkoxy, or cyano;
one of R₄ and R₆ is H, halo, optionally substituted C₁-C₄ alkyl, optionally substituted C₁-C₄ alkoxy, optionally substituted C₁-C₄ haloalkyl, optionally substituted C₁-C₄ haloalkoxy, or cyano; and the remaining R₄ or R₆ is

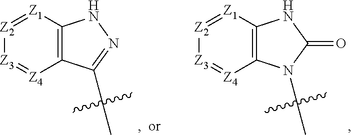

each of Z₁, Z₂, Z₃, and Z₄ is independently CR_{c} or N, in which R_{c} is H, halo, optionally substituted C₁-C₄ alkyl, optionally substituted C₁-C₄ alkoxy, optionally substituted C₁-C₄ haloalkyl, optionally substituted C₁-C₄ haloalkoxy, or cyano.

In some embodiments, R₄ or R₆ is

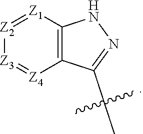

In some embodiments, the compound is a

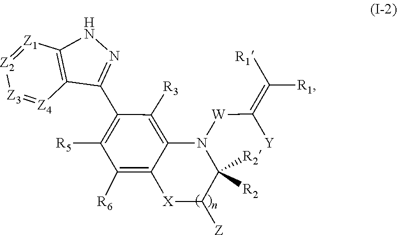

or a pharmaceutically acceptable salt thereof. In some embodiments, the compound is a compound of formula (I-3):

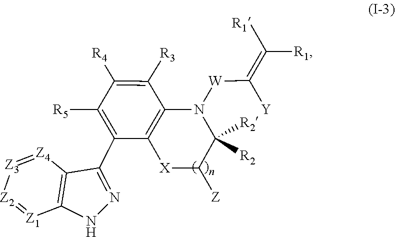

or a pharmaceutically acceptable salt thereof.

In some embodiments, R₄ or R₆ is

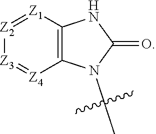

In some embodiments, the compound is a compound of formula (I-4):

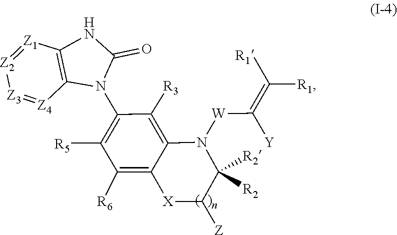

or a pharmaceutically acceptable salt thereof. In some embodiments, the compound is a compound of formula (I-5):

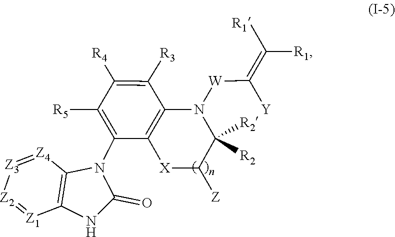

or a pharmaceutically acceptable salt thereof.

Exemplary compounds are provided in Table 1.

### Pharmaceutical Compositions

A pharmaceutical composition of the disclosure contains one or more of the compounds disclosed herein (e.g., one or more of the compounds of any one of formulas (I), (I-2), (I-3), (I-4), (I-5), and other compounds disclosed herein) as the therapeutic compound. In addition to a therapeutically effective amount of the compound, the pharmaceutical compositions also contain a pharmaceutically acceptable excipient, which can be formulated by methods known to those skilled in the art. The compounds disclosed herein (e.g., the compounds of formulas (I), (I-2), (I-3), (I-4), (I-5), and other compounds disclosed herein) may also be administered with or without other therapeutics for a particular condition, formulated in the same composition or different compositions for administration via the same or different routes.

The compounds disclosed herein (e.g., the compounds of formulas (I), (I-2), (I-3), (I-4), (I-5), and the compounds of Table 1) may be used in the form of free base, or in the form of salts or solvates. All forms are within the scope of the disclosure.

Exemplary routes of administration of the pharmaceutical compositions (or the compounds of the composition) include oral, sublingual, buccal, transdermal, intradermal, intramuscular, parenteral, intravenous, intra-arterial, intracranial, subcutaneous, intracerebroventricular, intraorbital, intraventricular, intrathecal (intraspinal), intraperitoneal, intranasal, inhalation, and topical administration.

### Formulations for Oral Administration

The pharmaceutical compositions of the disclosure include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers; granulating and disintegrating agents; binding agents; and lubricating agents, glidants, and antiadhesives. Other pharmaceutically acceptable excipients can be colorants, flavoring agents, plasticizers, humectants, buffering agents, and the like.

Pharmaceutical compositions for oral administration may also be presented as chewable tablets, as hard gelatin capsules where the active ingredient is mixed with an inert solid diluent, or as soft gelatin capsules where the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

The liquid forms in which the compounds and compositions of the present disclosure can be incorporated for administration orally include aqueous solutions, suitably flavored syrups, aqueous or oil suspensions, and flavored emulsions with edible oils, as well as elixirs and similar pharmaceutical vehicles.

Disorders Associated with MKK7-Related Pathway Regulation or Cellular Stress Pathways

### Neurological Disorders

Neurological disorders are disorders that affect the brain, as well as nerves throughout the body and also the spinal cord. Common symptoms of neurological disorders include numbness, tingling, muscle weakness, loss of muscle tone, loss of sensation, disruption or loss of autonomic function, numbness, bowel, or bladder incontinence, paralysis, confusion, pain, altered levels of consciousness, mood disorders, and sexual dysfunction. Certain primary symptoms, such as impaired movement and sensation, can further lead to secondary symptoms including muscle atrophy, loss of voluntary motor control and spasticity at sites of the body innervated by the neurological disorder, pressure (e.g., bed) sores, infections, and respiratory problems. Furthermore, cell death at the neurological disorder may continue long after the initial insult that precipitated the neurological disorder as a result of stress and inflammatory signaling that leads to further ischemia, inflammation, swelling, and disruption of synaptic signaling. Neurological disorder may result in total loss of motor and sensory function distal to the neurological disorder, or incomplete, resulting in partial loss of motor and sensory function.

Neurological disorders may present as various distinct conditions, depending on the site and severity of the condition. For example, peripheral neurological disorder results from damage to peripheral nerves that extend to the extremities of an individual, leading to numbness and/or loss of sensory function. Proximal neurological disorder results from damage to peripheral and/or central nerves, leading to muscle weakness in the upper part of the legs, buttocks, and/or hips in a subject. Autonomic neurological disorder results from damage and/or dysfunction of autonomic nerves that least to reduced and/or uncontrolled body homeostasis of an individual. Focal neurological disorder and/or polyneurological disorder results from damage to one nerve and/or a plurality of nerves, respectively. Central cord syndrome frequently results from damage to the cervical spinal cord, resulting in weakness in the upper extremities with relative sparing of function in the legs and spared sensation in sacral dermatomes (e.g., urinary sphincter, anal sphincter, and genitalia).

Examples of neurological disorders include, but are not limited to neurotraumatic disorders such as spinal cord injury (SCI), traumatic brain injury (TBI), stroke (e.g., hemorrhagic or ischemic stroke), peripheral nerve injury (PNI), myelopathy, hypoxic-ischemic encephalopathy, tumor-associated epilepsy, spasticity, multiple sclerosis, ischemia, amyotrophic lateral sclerosis (ALS), Parkinson's disease (PD), Alzheimer's disease (AD), and peripheral neuropathy (PN); neurodevelopmental disorders such as autism, Rett syndrome, Fragile X syndrome, Angelman syndrome, cerebral palsy, Down syndrome, pain (neuropathic pain, chronic pain, or inflammatory pain), Dravet syndrome, epilepsy (e.g., temporal lobe epilepsy), and sudden unexpected death in epilepsy); and affective disorders, such as schizophrenia, bipolar disorder, anxiety disorder, and major depressive disorder (MDD).

Neurotraumatic disorders are disorders of the nervous system that result from neurological trauma, such as, e.g., TBI, SCI, PNI, PN, stroke, ischemia, hypoxic-ischemic encephalopathy, tumor-associated epilepsy, and spasticity. In the U.S., roughly 1.7 million people are estimated to suffer TBI every year from causes such as falls, motor vehicle-related incidents, sports injuries, and violence, roughly 52,000 of which succumb to such injuries. Survivors of neurological trauma often face prolonged or indefinite disability.

TBI (also known as intracranial injury) usually results from an external force suddenly impacting the head of an individual, with the severity of the from mild (e.g., concussion) to severe (e.g., penetrating injury, coma-inducing injury). Sequalae of TBI often includes loss of consciousness, physical, cognitive, social, emotional, and behavioral impairments, but can also be fatal.

A SCI refers to any insult to the any region of the spinal cord, e.g., the cervical vertebrae, the thoracic vertebrae, the lumbar vertebrae, the sacral vertebrae, the sacrum, or the coccyx, that causes a negative effect on the function of the spinal cord, e.g., reduce mobility of feeling in limbs. The severity of a spinal cord injury is measured in levels of the injury's outcome, e.g., ranging from no effect on mobility, e.g., retained walking capacity, to paraplegia (e.g., paralysis of legs and lower region of body), and tetraplegia (e.g., loss of muscle strength in all four extremities).

PNI refers to any disorder resulting from a nerve injury caused by a traumatic event. Peripheral nerve injury is generally divided into three distinct events, namely, (1) Wallerian degeneration; (2) axon regeneration/growth; and (3) nerve innervation. Types of PNI include, from least severe to most severe: neurapraxia (axon remains intact, but myelin is damaged), axonotmesis (disruption of the axon with maintenance of the epineurium), and neurotmesis (loss of axon continuity/axon transection).

Stroke is a condition which occurs when the blood supply to a part of the brain is interrupted (i.e., ischemic stroke) by obstruction of a blood vessel by a blood clot, an embolism, systemic hypoperfusion, or cerebral venous sinus thrombosis or when a blood vessel in the brain bursts and releases blood into the spaces surrounding the brain cells (i.e., hemorrhagic stroke) as a result of an intracerebral or a subarachnoid hemorrhage. Stroke poses a substantial public burden as nearly 77.2 million people experienced an ischemic stroke, and 29.1 million people experienced a hemorrhagic stroke in 2019.

Depending on the area of the brain affected by the stroke, the symptoms of a stroke may include numbness or weakness, especially on one side of the body corresponding to the contralateral side of the stroke, muscle flaccidity or spasticity, confusion, trouble understanding or producing speech, impaired vision in both eyes, impaired mobility, dizziness, severe headache, or loss of balance or coordination.

Neurological trauma may also result from progressive neurodegenerative disorders that result in damage to neural tissue of the CNS. Non-limiting examples of neurodegenerative disorders contemplated for treatment using the presently disclosed compositions and methods include, but are not limited to, ALS, PD, AD, and PN.

Neurodevelopmental disorders refer to neurological disorders resulting from abnormal development of the nervous system and are characterized by abnormal brain function, including, but not limited to, impairments in emotional regulation, learning and memory, impulse control, and cognition. This class of neurological disorders is characterized by diverse etiologies that may account for the multeity of symptoms and their degree of severity. Generally, neurodevelopmental disorders are caused by disruptions the neurotypical developmental trajectory of the nervous system, which can produce pathological anatomical architecture and connectivity in the nervous system. Causes of neurodevelopmental disorders may include genetic and metabolic diseases, social isolation, inflammatory and autoimmune disorders, infectious diseases, malnutrition, physical trauma, as well as environmental factors. The present disclosure contemplates treatment of neurodevelopmental disorders such as, e.g., autism spectrum disorders, Rett syndrome, Fragile X syndrome, Angelman syndrome, cerebral palsy, Down syndrome, pain (e.g., neuropathic pain, chronic pain, or inflammatory pain), Dravet syndrome, epilepsy (e.g., epilepsy related to one or more KCC2 mutations or epilepsy of infancy with migrating focal seizures (EIMFS) or temporal lobe epilepsy), and sudden unexpected death in epilepsy by administering a composition of the disclosure to the afflicted subject, thereby treating the subject.

Affective disorders (also known as mood disorders) are a class of neurological conditions characterized by dysregulation of normal affect and mood. Disorders of affect may feature mania or hypomania (e.g., schizophrenia and bipolar disorder), depressed mood (e.g., schizophrenia, bipolar disorder, and MDD), and moods that cycle between mania and depression (e.g., bipolar disorder). Affective disorders that may be treated using the disclosed methods and compositions include schizophrenia, bipolar disorder, and MDD.

Schizophrenia is a psychiatric disease characterized by recurrent psychosis. Symptoms of schizophrenia may include (1) positive symptoms related to hallucinations and reality distortion; (2) disorganized symptoms characterized by attentional impairment and thought disorder; and (3) negative symptoms such as apathy, anhedonia, avolition and loss of verbal fluency. Dysfunction of the limbic-cortical system may be implicated in all three types of symptoms. Causes of schizophrenia have been attributed to biological sex, genetic mutations, environmental factors, malnutrition during pregnancy, and age of parents, among other factors. Several hypotheses exist as to the etiology of schizophrenia, one being the glutamate hypothesis in which reduced glutamatergic drive to inhibitory interneurons is thought to result in reduced cortical inhibition and altered cortical network dynamics that lead to presentation of clinical symptoms.

Bipolar disorder is an affective disorder that features recurrent bouts of depression and mania (i.e., abnormally elevated mood) spanning from days to weeks each. Causes of bipolar disorder may be manifold, but genetic and environmental factors have been implicated. Generally, two types of bipolar disorder exist, namely, bipolar I disorder, in which there has been at least one manic episode with or without depressive episodes, and bipolar II disorder, in which there has been at least one hypomanic episode and one major depressive episode.

MDD is a neurological disorder that is often characterized by the patient having at least two weeks of sustained low mood, low self-esteem, loss of interest in routine activities, hyperalgesia, and low psychomotor activity. Depression in MDD may last for periods of time (weeks, days, months, or years) separated by years or may be continuous. MDD may pose a substantial risk to the afflicted patient as the patient may be at a substantially higher risk for suicide. Etiological causes of the disorder have been attributed to substance abuse, other medical conditions (e.g., neurological disorders, metabolic disorders, gastrointestinal disorders, endocrine disorders, cardiovascular disease, pulmonary disease, cancer, and autoimmune disease), and genetic and environmental factors.

A neurological disorder may also be caused by infection, ischemia, and tumors. Owing to the physiological barriers to regeneration in the central nervous system (CNS), neurological disorders have been a notoriously difficult condition to treat, with most treatments being palliative and rehabilitative. Most treatments involve imposing limitations to movement, maintenance of proper blood pressure by frequent repositioning of the subject, and physical and occupation therapy.

### Cancer Disorders

In some embodiments of any of the above aspects, the composition may treat one or more cancer disorders, including one or more conditions characterized by unregulated or abnormal cell growth. The abnormal cell growth may be the result of a cancer cell, which refers to an abnormal cell, mass, or population of cells that result from excessive division that may be malignant or benign and all pre-cancerous and cancerous cells and tissues. In some embodiments, the cancer disorder is a solid tumor (e.g., Melanoma, small cell lung cancer, non-small cell lung cancer, gastric cancer, colorectal cancer, head and neck cancer, ovarian cancer, kidney cancer, prostate cancer, breast cancer, hepatocellular carcinoma, or pancreatic cancer), a cancer that is treated with immunotherapy (e.g., melanoma, non-small cell lung cancer, kidney cancer, renal cell carcinoma, bladder cancer, head and neck cancer, Hodgkin's lymphoma, leukemia, urothelial carcinoma, gastric cancer, microsatellite instability-high cancer, colorectal cancer, or hepatocellular carcinoma), or a cancer that does not respond to immunotherapy (e.g., a cancer that does not respond to immunotherapy or a cancer that did not respond to prior treatment with immunotherapy, e.g., a cancer for which immunotherapy is not effective).

The cancer may be any solid or liquid cancer and includes benign or malignant tumors, and hyperplasias, including gastrointestinal cancer (such as non-metastatic or metastatic colorectal cancer, pancreatic cancer, gastric cancer, esophageal cancer, hepatocellular cancer, cholangiocellular cancer, oral cancer, lip cancer); urogenital cancer (such as hormone sensitive or hormone refractory prostate cancer, renal cell cancer, bladder cancer, penile cancer); gynecological cancer (such as ovarian cancer, cervical cancer, endometrial cancer); lung cancer (such as small-cell lung cancer and non-small-cell lung cancer); head and neck cancer (e.g., head and neck squamous cell cancer); CNS cancer including malignant glioma, astrocytomas, retinoblastomas and brain metastases; malignant mesothelioma; non-metastatic or metastatic breast cancer (e.g., hormone refractory metastatic breast cancer); skin cancer (such as malignant melanoma, basal and squamous cell skin cancers, Merkel Cell Carcinoma, lymphoma of the skin, Kaposi Sarcoma); thyroid cancer; bone and soft tissue sarcoma; and hematologic neoplasias (such as multiple myeloma, acute myelogenous leukemia, chronic myelogenous leukemia, myelodysplastic syndrome, acute lymphoblastic leukemia, Hodgkin's lymphoma).

Additional cancers include breast cancer, lung cancer, stomach cancer, colon cancer, liver cancer, renal cancer, colorectal cancer, prostate cancer, pancreatic cancer, cervical cancer, anal cancer, vulvar cancer, penile cancer, vaginal cancer, testicular cancer, pelvic cancer, thyroid cancer, uterine cancer, rectal cancer, brain cancer, head and neck cancer, esophageal cancer, bronchus cancer, gallbladder cancer, ovarian cancer, bladder cancer, oral cancer, oropharyngeal cancer, larynx cancer, biliary tract cancer, skin cancer, a cancer of the central nervous system, a cancer of the respiratory system, and a cancer of the urinary system. Examples of breast cancers include, but are not limited to, triple-negative breast cancer, triple-positive breast cancer, HER2-negative breast cancer, HER2-positive breast cancer, estrogen receptor-positive breast cancer, estrogen receptor-negative breast cancer, progesterone receptor-positive breast cancer, progesterone receptor-negative breast cancer, ductal carcinoma in situ (DCIS), invasive ductal carcinoma, invasive lobular carcinoma, inflammatory breast cancer, Paget disease of the nipple, and phyllodes tumor.

Other cancers include leukemia (e.g., B-cell leukemia, T-cell leukemia, acute myeloid leukemia (AML), chronic myeloid leukemia (CML), acute lymphocytic (lymphoblastic) leukemia (ALL), chronic lymphocytic leukemia (CLL), and erythroleukemia), sarcoma (e.g., angiosarcoma, chondrosarcoma, Ewing's sarcoma, fibrosarcoma, gastrointestinal stromal tumor, leiomyosarcoma, liposarcoma, malignant peripheral nerve sheath tumor, malignant fibrous cytoma, osteosarcoma, pleomorphic sarcoma, rhabdomyosarcoma, synovial sarcoma, vascular sarcoma, Kaposi's sarcoma, dermatofibrosarcoma, epithelioid sarcoma, leyomyosarcoma, and neurofibrosarcoma), carcinoma (e.g., basal cell carcinoma, large cell carcinoma, small cell carcinoma, non-small cell lung carcinoma, renal carcinoma, hepatocarcinoma, gastric carcinoma, choriocarcinoma, adenocarcinoma, hepatocellular carcinoma, giant (or oat) cell carcinoma, squamous cell carcinoma, adenosquamous carcinoma, anaplastmic carcinoma, adrenocortical carcinoma, cholangiocarcinoma, Merkel cell carcinoma, ductal carcinoma in situ (DCIS), and invasive ductal carcinoma), blastoma (e.g., hepatoblastoma, medulloblastoma, nephroblastoma, neuroblastoma, pancreatoblastoma, pleuropulmonary blastoma, retinoblastoma, and glioblastoma multiforme), lymphoma (e.g., Hodgkin's lymphoma, non-Hodgkin's lymphoma, and Burkitt lymphoma), myeloma (e.g., multiple myeloma, plasmacytoma, localized myeloma, and extramedullary myeloma), melanoma (e.g., superficial spreading melanoma, nodular melanoma, lentigno maligna melanoma, acral lentiginous melanoma, and amelanotic melanoma), neuroma (e.g., ganglioneuroma, Pacinian neuroma, and acoustic neuroma), glioma (e.g., astrocytoma, oligoastrocytoma, ependymoma, brainstem glioma, optic nerve glioma, and oligoastrocytoma), pheochromocytoma, meningioma, malignant mesothelioma, and virally induced cancer.

In some embodiments, the cancer is a paraneoplastic cancer (e.g., a cancer that causes a paraneoplastic syndrome). Paraneoplastic syndromes are rare disorders that are triggered by an altered immune system response to a neoplasm, and are mediated by humoral factors such as hormones, cytokines, or auto-antibodies produced by the tumor. Symptoms of paraneoplastic syndrome may be endocrine, neuromuscular, or musculoskeletal, cardiovascular, cutaneous, hematologic, gastrointestinal, renal, or neurological. Paraneoplastic syndromes commonly present with lung, breast, and ovarian cancer and cancer of the lymphatic system (e.g., lymphoma). Paraneoplastic neurological disorders are disorders that affect the central or peripheral nervous system, and can include symptoms such as ataxia (difficulty with walking and balance), dizziness, nystagmus (rapid uncontrolled eye movements), difficulty swallowing, loss of muscle tone, loss of fine motor coordination, slurred speech memory loss, vision problems, sleep disturbances, dementia, seizures, or sensory loss in the limbs. Breast, ovarian, and lung cancers are most commonly associated with paraneoplastic neurological disorders. Other common types of paraneoplastic syndromes include paraneoplastic cerebellar degeneration, paraneoplastic pemphigus, paraneoplastic autonomic neuropathy, paraneoplastic encephalomyelitis, and cancer-associated autoimmune retinopathy.

Endocrine paraneoplastic syndromes include Cushing syndrome (caused by ectopic ACTH), which is most commonly caused by small cell lung cancer, pancreatic carcinoma, neural tumors, or thymoma; SIADH (caused by antidiuretic hormone), which is most commonly caused by small cell lung cancer and CNS malignancies; hypercalcemia (caused by PTHrp, TGFα, TNF, or IL-1), which is most commonly caused by lung cancer, breast carcinoma, renal and bladder carcinoma, multiple myeloma, adult T cell leukemia/lymphoma, ovarian carcinoma, and squamous cell carcinoma (e.g., lung, head, neck, or esophagus carcinoma); hyperglycemia (caused by insulin insulin-like substance, or “big” IGF-II), which is most commonly caused by fibrosarcoma, mesenchymal sarcomas, insulinoma, and hepatocellular carcinoma; carcinoid syndrome (caused by serotonin or bradykinin), which is most commonly caused by bronchial adenoma, pancreatic carcinoma, and gastric carcinoma; and hyperaldosteronism (caused by aldosterone), which is most commonly caused by adrenal adenoma/Conn's syndrome, non-Hodgkin's lymphoma, ovarian carcinoma, and pulmonary cancer.

Neurological paraneoplastic syndromes include Lambert-Eaton myasthenic syndrome (LEMS), which is most commonly caused by small cell lung cancer; paraneoplastic cerebellar degeneration, which is most commonly caused by lung cancer, ovarian cancer, breast carcinoma, and Hodgkin's lymphoma; encephalomyelitis; limbic encephalitis, which is most commonly caused by small cell lung carcinoma; myasthenia gravis, which is most commonly caused by thymoma; brainstem encephalitis; opsoclonus myoclonus ataxia (caused by autoimmune reaction against Nova-1), which is most commonly caused by breast carcinoma, ovarian carcinoma, small cell lung carcinoma, and neuroblastoma; anti-NMDA receptor encephalitis (caused by autoimmune reaction against NMDAR subunits), which is most commonly caused by teratoma; and polymyositis, which is most commonly caused by lung cancer, bladder cancer, and non-Hodgkin's lymphoma. Mucotaneous paraneoplastic syndromes include acanthosis nigricans, which is most commonly caused by gastric carcinoma, lung carcinoma, and uterine carcinoma; dermatomyositis, which is most commonly caused by bronchogenic carcinoma, breast carcinoma, ovarian cancer, pancreatic cancer, stomach cancer, colorectal cancer, and Non-Hodgkin's lymphoma; Leser-Trelat sign; necrolytic migratory erythema, which is most commonly caused by glucoganoma; Sweet's syndrome; florid cutaneous papillomatosis; pyoderma gangrenosum; and acquired generalized hypertrichosis.

Hematological syndromes include granulocytosis (caused by G-CSF); polycythemia (caused by erythropoietin), which is commonly caused by renal carcinoma, cerebellar hemangioma, and heptatocellular carcinoma; Trousseau sign (caused by mucins), which is commonly caused by pancreatic carcinoma and bronchogenic carcinoma; nonbacterial thrombotic endocarditis, which is caused by advanced cancers; and anemia, which is most commonly caused by thymic neoplasms. Other paraneoplastic syndromes include membranous glomerular nephritis; neoplastic fever; Staffer syndrome, which is caused by renal cell carcinoma; and tumor-induced osteomalacia (caused by FGF23), which is caused by hemangiopericytoma and phosphaturic mesenchymal tumor.

The cancer may be highly innervated, metastatic, non-metastatic cancer, or benign (e.g., a benign tumor). The cancer may be a primary tumor or a metastasized tumor.

### Inflammatory Disorders

In some embodiments of any of the above aspects, the composition may treat one or more inflammatory disorder, including one or more conditions characterized by unregulated immune system responses. The unregulated immune system responses may be the result of an individual's immune system attacking one or more self-cells present within the individual's body. The unregulated immune system responses may lead to one or more cardinal signs of inflammation, such as redness, swelling, heat, pain, or loss of function.

In some embodiments, the inflammatory disorder is ankylosing spondylitis, antiphospholipid antibody syndrome, gout, inflammatory arthritis center, myositis, rheumatoid arthritis, scleroderma, Sjogren's syndrome, systemic lupus erythematosus, vasculitis, or the like. In some embodiments, the inflammatory disorders may be the result of one or more autoimmune diseases or such as unregulated inflammation resulting from fatty liver disease, endometriosis, type 2 diabetes mellitus, type 1 diabetes mellitus, inflammatory bowel disease, asthma, obesity, Alzheimer's disease, Parkinson's disease, or the like.

### Methods

### Inhibiting an MKK7 Enzyme

The compounds disclosed herein (e.g., the compounds of formulas (I), (I-2), (I-3), (I-4), (I-5), and other compounds disclosed herein) are, in general, suitable for use in inhibiting an MKK7 enzyme. MKK7 enzymes belong to a member of the mitogen-activated protein kinase kinase family, in which the protein has six distinct isoforms, three possible N-termini, and two possible C-termini. MKK7 enzymes are involved in the stress-activated protein kinase (SAP)/JNK signaling pathway, in which MKK7 enzymes phosphorylate JNK isoforms. The compounds disclosed herein may irreversibly bind to one or more cysteine residues of the MKK7 enzyme via covalent bonding and inhibit the enzyme from operating in the SAP/JNK pathway.

### Increasing Survival

The compounds disclosed herein (e.g., the compounds of formulas (I), (I-2), (I-3), (I-4), (I-5), and other compounds disclosed herein) are, in general, suitable for use in increasing survival, or reducing death or degeneration of a damaged or degenerating neuron. A neuron or cell may be degenerating or declining when there is a reduced likelihood of cell viability, which may be measured by one or more cell viability measurements, e.g., metabolic assays (e.g., MTT assay, XTT assay, etc.), ATP measurement assays, etc. Degeneration may be determined by determining a number of cells undergoing apoptosis via an apoptosis assay, annexin V assay, caspase assay, chromatin condensation assay, TUNEL assay, cytochrome c release assay, etc. The compounds disclosed herein (e.g., the compounds of formulas (I), (I-2), (I-3), (I-4), (I-5), and other compounds disclosed herein) may increase survival by irreversibly binding to an MKK7 enzyme. The irreversibly bound compound to MKK7 may inhibit the MKK7, in which inhibition of MKK7 may reduce apoptosis signaling along the JNK signaling pathway.

Treating a Disorder Associated with MKK7-Related Pathway Regulation or Cellular Stress Pathways

The compounds disclosed herein (e.g., the compounds of formulas (I), (I-2), (I-3), (I-4), (I-5), and other compounds disclosed herein) are, in general, suitable for use in treating a disorder associated with MKK7-related pathway regulation or cellular stress pathways. The disorder may be associated with an MKK7 enzyme. Non-limiting examples of disorders associated with an MKK7 enzyme include a neurodegenerative disease, a neurotraumatic, neurodevelopmental, and/or affective disorder, or complications resulting therefrom. Non-limiting examples of neurodegenerative diseases include glaucoma, neuroblastoma, glioblastoma, and lysosomal storage disorders. Non-limiting examples of neurotraumatic disorders include spinal cord injury (SCI), optic nerve injury, traumatic brain injury (TBI), stroke (e.g., hemorrhagic or ischemic stroke), peripheral nerve injury (PNI), multiple sclerosis (MS), ischemia, amyotrophic lateral sclerosis (ALS), Parkinson's disease (PD), Alzheimer's disease (AD), peripheral neuropathy (PN), hypoxic-ischemic encephalopathy, tumor-associated epilepsy, chemotherapy-induced peripheral neuropathy, and spasticity. Neurodevelopmental disorders may include, but are not limited to autism, Rett syndrome, Fragile X syndrome, Angelman syndrome, cerebral palsy, Down syndrome, pain (e.g., neuropathic pain, chronic pain, or inflammatory pain), Dravet syndrome, epilepsy (e.g., epilepsy related to one or more KCC2 mutations or epilepsy of infancy with migrating focal seizures (EIMFS) or temporal lobe epilepsy), and sudden unexpected death in epilepsy. Non-limiting examples of affective disorders include schizophrenia, bipolar disorder, anxiety disorder, and major depressive disorder (MDD).

The methods described herein can be used to treat cancer disorders or inflammatory disorders in a subject by administering to the subject an effective amount of any of the compositions described herein.

The methods described herein can also be used to potentiate or increase an MKK7-related pathway or cellular stress pathways in a subject in need thereof. For example, the subject has cancer, such as a cancer described herein.

The compositions described herein may inhibit proliferation or disrupt the function of non-neural cells that promote cancer growth that are associated with the cancer, e.g., the method includes administering to the subject an effective amount of the compositions described herein for a time sufficient to inhibit proliferation or disrupt the function of non-neural cells that promote cancer growth that are associated with the cancer. Non-neural cells that promote cancer growth that are associated with the cancer include malignant cancer cells, malignant cancer cells in necrotic and hypoxic areas, M2 macrophages, tumor associated macrophages, T regulatory cells, myeloid derived suppressor cells, adipocytes, B10 cells, B regulatory cells, endothelial cells, cancer associated fibroblasts, fibroblasts, mesenchymal stem cells, red blood cells, or extracellular matrix. The proliferation of non-neural cells that promote cancer growth that are associated with the cancer may be decreased in the subject at least 1%, 2%, 5%, 10%, 15%, 20%, 25%, 30%, 35%, 40%, 50%, 60%, 70%, 80%, 90%, 95% or more, compared to before the administration. The proliferation of non-neural cells that promote cancer growth that are associated with the cancer can be decreased in the subject between 5-20%, between 5-50%, between 10-50%, between 20-80%, between 20-70%.

The compositions described herein may promote proliferation or enhance the function of non-neural cells that disrupt cancer growth that are associated with the cancer, e.g., the method includes administering to the subject an effective amount of any one of the compositions described herein for a time sufficient to promote proliferation or enhance the function of non-neural cells that disrupt cancer growth that are associated with the cancer. Non-neural cells that disrupt cancer growth that are associated with the cancer include NK cells, NKT cells, M1 macrophages, TH1 helper cells, TH2 helper cells, CD8 cytotoxic T cells, TH17 cells, tumor associated neutrophils, terminally differentiated myeloid dendritic cells, T lymphocytes, B lymphocytes, lymphatic endothelial cells, pericytes, dendritic cells, mesenchymal stem cells, red blood cells, or extracellular matrix. The proliferation of non-neural cells that disrupt cancer growth that are associated with the cancer may be increased in the subject at least 1%, 2%, 5%, 10%, 15%, 20%, 25%, 30%, 35%, 40%, 50%, 60%, 70%, 80%, 90%, 95% or more, compared to before the administration. The proliferation of non-neural cells that disrupt cancer growth that are associated with the cancer can be increased in the subject between 5-20%, between 5-50%, between 10-50%, between 20-80%, between 20-70%.

The composition can be administered in an amount sufficient to treat cancer. For example, the stroma associated with the tumor, e.g., fibroblasts, is disrupted such that an essential function, e.g., the production of matrix metalloproteases, is altered to inhibit tumor survival or promote tumor control.

The composition can have one or more of the following activities: (a) inhibits an immune checkpoint, (b) activates anti-tumor immune response, (c) activate tumor-specific T cells from draining lymph nodes, and/or (d) stimulates a neoantigen-specific immune response. The activity can be modulated as appropriate in the subject (e.g., a human subject or animal model) at least 1%, 2%, 5%, 10%, 15%, 20%, 25%, 30%, 35%, 40%, 50%, 60%, 70%, 80%, 90%, 95% or more, compared to before the administration. The activity can be modulated as appropriate in the subject between 5-20%, between 5-50%, between 10-50%, between 20-80%, between 20-70%.

The composition can treat cancer by increasing cancer cell death in a subject (e.g., a human subject or animal model) or in a cancer cell culture (e.g., a culture generated from a patient tumor sample, a cancer cell line, or a repository of patient samples). Any one of the compositions described herein can increase cancer cell death by at least 1%, 2%, 5%, 10%, 15%, 20%, 25%, 30%, 35%, 40%, 50%, 60%, 70%, 80%, 90%, 95% or more compared to before administration to a subject or cancer cell culture. Any one of the compositions described herein can increase cancer cell death in a subject or cancer cell culture between 5-20%, between 5-50%, between 10-50%, between 20-80%, between 20-70%.

The composition can also act to inhibit cancer cell growth, proliferation, metastasis, migration, or invasion, e.g., the method includes administering to the subject (e.g., a human subject or animal model) or a cancer cell culture (e.g., a culture generated from a patient tumor sample, a cancer cell line, or a repository of patient samples) any one of the compositions described herein in an amount (e.g., an effective amount) and for a time sufficient to inhibit cancer cell growth, proliferation, metastasis, migration, or invasion. Cancer cell growth, proliferation, metastasis, migration, or invasion can be decreased in the subject or cancer cell culture at least 1%, 2%, 5%, 10%, 15%, 20%, 25%, 30%, 35%, 40%, 50%, 60%, 70%, 80%, 90%, 95% or more, compared to before the administration Cancer cell growth, proliferation, metastasis, migration, or invasion can be decreased in the subject or cancer cell culture between 5-20%, between 5-50%, between 10-50%, between 20-80%, between 20-70%.

### Composition and Dosage

The dosage of the pharmaceutical compositions of the disclosure depends on factors including, but are not limited to, the route of administration, the severity of the condition to be treated, and physical characteristics, e.g., age, weight, and general health, of the subject. Typically, the amount of a compound disclosed herein (e.g., a compound of any one of formulas (I), (I-2), (I-3), (I-4), (I-5), and other compounds disclosed herein) contained within a single dose may be an amount that effectively imparts the desired therapeutic effect without inducing significant toxicity. The dosage may be adapted by the clinician in accordance with conventional factors such as the extent of the disease and different parameters of the subject.

Pharmaceutical compositions of the disclosure that contain a compound disclosed herein (e.g., a compound of any one of formulas (I), (I-2), (I-3), (I-4), (I-5), and other compounds disclosed herein may be administered to a subject in need thereof one or more times (e.g., 10 times or more) daily, or as medically necessary. The timing between administrations may decrease as the medical condition improves or increase as the health of the subject declines.

The following examples are merely illustrative and should not be construed as limiting the scope of this disclosure in any way as many variations and equivalents will become apparent to those skilled in the art upon reading the present disclosure. The contents of all references, patents, and patent applications cited throughout this application are expressly incorporated herein by reference.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. Synthesis of 1-[6-(6-methyl-1H-indazol-3-yl)-2,3-dihydroindol-1-yl]prop-2-en-1-one (Compound 4)

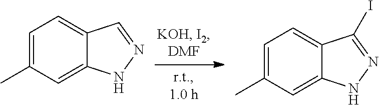

A solution of 6-methyl-1H-indazole (5.00 g, 38 mmol, 1.00 equiv), KOH (5.30 g, 94.58 mmol, 2.50 equiv) and I₂ (19.24 g, 75.76 mmol, 2 equiv) in DMF (80 mL) was stirred for 1.0 h at room temperature. The reaction mixture was subsequently poured into 200 mL ice/sat. Na₂S₂O₃ (aq.) solution. The solid was filtered out and washed by water (50 mL) and dried under vacuum to provide 3-iodo-6-methyl-1H-indazole (9.0 g, 83% yield) as an off-white solid.

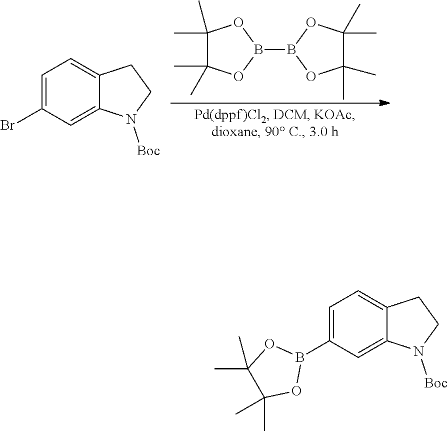

To a stirred solution of tert-butyl 6-bromo-2,3-dihydroindole-1-carboxylate (1.00 g, 3.4 mmol, 1.0 equiv) in dioxane (30 mL) was added bis(pinacolato)diboron (1.3 g, 5.0 mmol, 1.5 equiv), Pd(dppf)Cl₂·DCM (250 mg, 0.34 mmol, 0.10 equiv), and KOAc (990 mg, 10 mmol, 3.0 equiv) at room temperature under nitrogen atmosphere. The resulting mixture was stirred for 3.0 h at 90° C. under nitrogen atmosphere. The mixture was allowed to cool down to room temperature. The reaction was quenched by the addition of water (10 mL) at 0° C. The resulting mixture was extracted with EtOAc (3×10 mL). The combined organic layers were washed with water (2×5 mL) and dried over anhydrous Na₂SO₄. After filtration, the filtrate was concentrated under reduced pressure to provide tert-butyl 6-(4,4,5,5-tetramethyl-1,3,2-dioxaborolan-2-yl)-2,3-dihydroindole-1-carboxylate (1.0 g, 73%) as a black solid. The crude product was used in the next step directly without further purification.

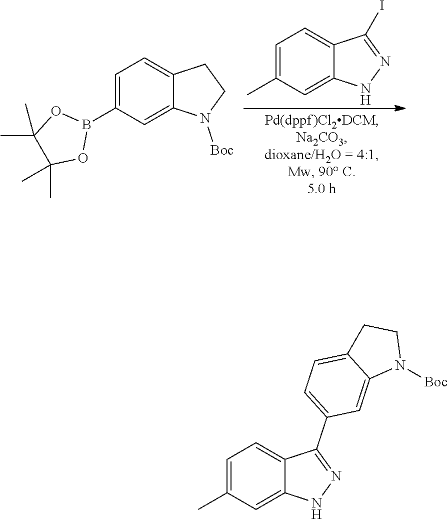

To a solution of tert-butyl 6-(4,4,5,5-tetramethyl-1,3,2-dioxaborolan-2-yl)-2,3-dihydroindole-1-carboxylate (270 mg, 0.78 mmol, 1.0 equiv) and 3-iodo-6-methyl-1H-indazole (200 mg, 0.78 mmol, 1.0 equiv) in dioxane (4.0 mL) and H₂O (1.0 mL) were added Na₂CO₃ (410 mg, 3.9 mmol, 5.0 equiv) and Pd(dppf)Cl₂·CH₂Cl₂ (64 mg, 0.08 mmol, 0.10 equiv). The final reaction mixture was irradiated with microwave radiation for 5.0 h at 90° C. After reaction, the resulting mixture was filtered, and the filter cake was washed with EtOAc (3×10 mL). The filtrate was concentrated under reduced pressure. The residue was purified by silica gel column chromatography, eluted with PE/EA (3:1) to afford tert-butyl 6-(6-methyl-1H-indazol-3-yl)-2,3-dihydroindole-1-carboxylate (200 mg, 66%) as a brown solid.

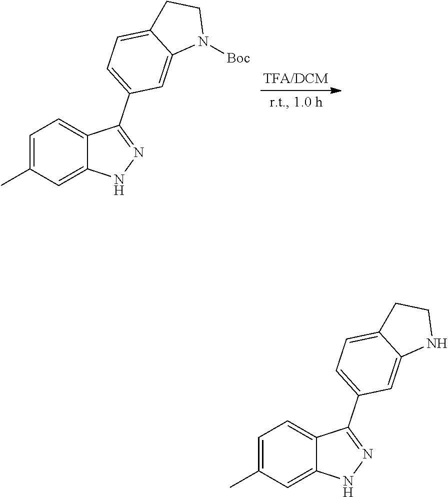 [SMILES: Cc1ccc2c(-c3ccc4c(c3)N(C(=O)OC(C)(C)C)CC4)n[nH]c2c1 ; Cc1ccc2c(-c3ccc4c(c3)NCC4)n[nH]c2c1]

To a stirred solution of tert-butyl 6-(6-methyl-1H-indazol-3-yl)-2,3-dihydroindole-1-carboxylate (100 mg, 0.29 mmol, 1.0 equiv) in DCM (1.0 mL) was added TFA (0.50 mL) dropwise at 0° C. The resulting mixture was stirred for 1.0 h at room temperature. After reaction, the resulting mixture was concentrated under reduced pressure to provide (3-(2,3-dihydro-1H-indol-6-yl)-6-methyl-1H-indazole) (100 mg, crude) as a brown oil. The crude product was used in the next step directly without further purification.

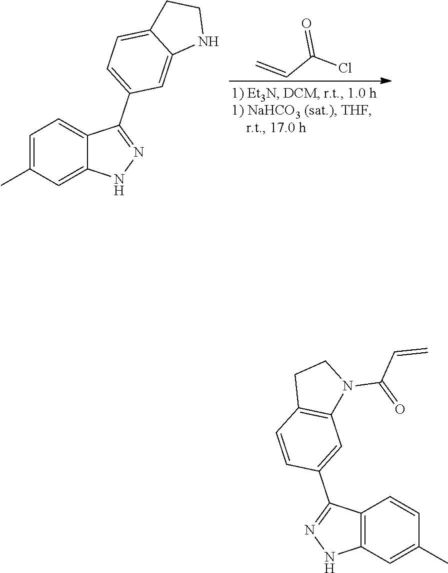

To a stirred solution of 3-(2,3-dihydro-1H-indol-6-yl)-6-methyl-1H-indazole (100 mg, 0.40 mmol, 1.0 equiv) in DCM (1.0 mL) was added acryloyl chloride (36 mg, 0.40 mmol, 1.0 equiv) at 0° C. The resulting mixture was stirred for 1.0 h at room temperature. Afterwards, the resulting mixture was concentrated under reduced pressure. The residue was dissolved in THE (1.0 mL). To the above mixture was added sat. NaHCO₃ (aq. 1 mL) dropwise at 0° C. The resulting mixture was stirred for additional overnight at room temperature. After reaction, the resulting mixture was concentrated under reduced pressure. The residue was dissolved in DCM (5 mL). The resulting mixture was filtered, the filter cake was washed with DCM (3×5 mL). The filtrate was concentrated under reduced pressure. The residue was purified by Prep-TLC (PE/EA 1:1) to afford 1-[6-(6-methyl-1H-indazol-3-yl)-2,3-dihydroindol-1-yl]prop-2-en-1-one (60 mg) as a light yellow solid, which was then was dissolved in DMF (1.0 mL). The solution was filtered and the filtration in DMF (1.0 mL) was purified by Prep-HPLC: Column: XBridge Shield RP18 OBD Column, 30*150 mm, 5 μm; Mobile Phase A: Water(10 mmol/L NH₄HCO₃), Mobile Phase B: ACN; Flow rate: 60 mL/min; Gradient: 32% B to 55% B in 7 min, 55% B; wavelength: 254 nm; RT1(min): 6.83; Number Of Runs: 0. The fractions were combined and lyophilized directly to afford 1-[6-(6-methyl-1H-indazol-3-yl)-2,3-dihydroindol-1-yl]prop-2-en-1-one (27.8 mg, 22%) as a white solid.

LC/MS: mass calcd. For C₁₉H₁₇N₃O: 303.14, found: 304.15 [M+H]⁺.

¹H NMR (300 MHz, DMSO-d6) δ: 12.97 (br s, 1H), 8.74 (s, 1H), 7.90 (d, J=8.4 Hz, 1H), 7.65 (d, J=7.5 Hz, 1H), 7.37 (m, 2H), 7.06 (d, J=8.7 Hz, 1H), 6.75-6.84 (m, 1H), 6.32-6.38 (m, 1H), 5.83-5.87 (m, 1H), 4.28 (t, J=8.1 Hz, 2H), 3.22 (t, J=8.4 Hz, 2H), 2.46 (s, 3H).

### Example 2. Synthesis of 1-[7-(6-methyl-1H-indazol-3-yl)-3,4-dihydro-2H-quinolin-1-yl]prop-2-en-1-one (Compound 12)

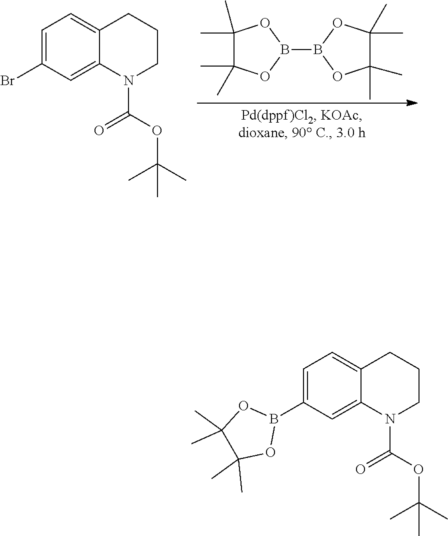

Into a 100 mL flask, tert-butyl 7-bromo-3,4-dihydro-2H-quinoline-1-carboxylate (500 mg, 1.6 mmol, 1.0 equiv), bis(pinacolato)diboron (610 mg, 2.4 mmol, 1.5 equiv), KOAc (320 mg, 3.2 mmol, 2.0 equiv), and Pd(dppf)Cl₂CH₂Cl₂ (130 mg, 0.16 mmol, 0.10 equiv), dioxane (15 mL) was added. The reaction was stirred at 90° C. for 3.0 h under nitrogen atmosphere and quenched with water (30 mL) at room temperature. The resulting mixture was extracted with EtOAc (3×30 mL). The combined organic layers were washed with brine (2×20 mL) and dried over anhydrous Na₂SO₄. After filtration, the filtrate was concentrated under reduced pressure, resulting in tert-butyl-7-(4,4,5,5-tetramethyl-1,3,2-dioxaborolan-2-yl)-3,4-dihydro-2H-quinoline-1-carboxylate (970 mg, crude) as a black solid.

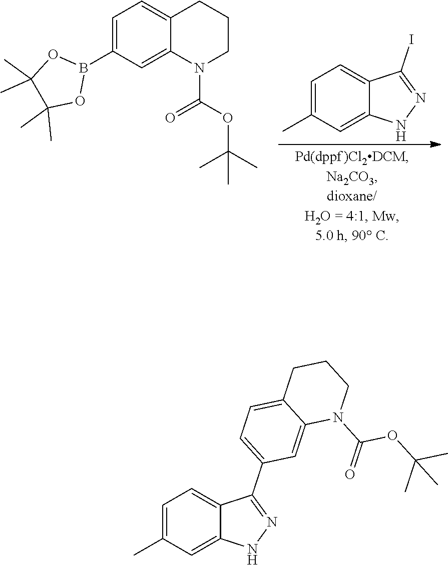

Tert-butyl 7-(4,4,5,5-tetramethyl-1,3,2-dioxaborolan-2-yl)-3,4-dihydro-2H-quinoline-1-carboxylate (600 mg, 1.7 mmol, 1.5 equiv), 3-iodo-6-methyl-1H-indazole (290 mg, 1.1 mmol, 1.0 equiv), Na₂CO₃ (360 mg, 3.4 mmol, 3.0 equiv), Pd(dppf)Cl₂·CH₂Cl₂ (91 mg, 0.11 mmol, 0.10 equiv), dioxane (12 mL), and H₂O (3.0 mL) was added in a 40 mL tube and the reaction was stirred at 90° C. under N₂ atmosphere and under Mw. for 5.0 h. The reaction mixture was filtered and washed with MeOH, the organic phase was concentrated. The residue was purified by silica gel column chromatography, and eluted with PE/EA (3:1) to afford tert-butyl 7-(6-methyl-1H-indazol-3-yl)-3,4-dihydro-2H-quinoline-1-carboxylate (310 mg, 62% yield) as a yellow oil.

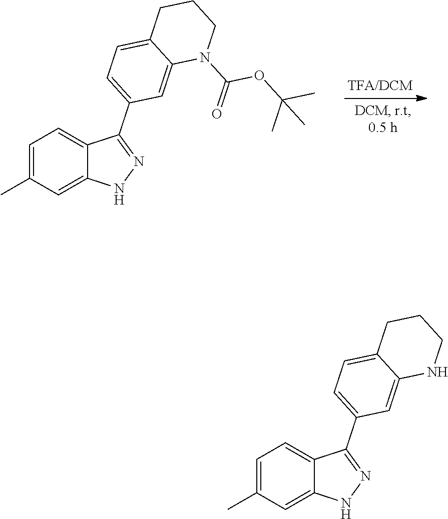

Into a 25 mL flask was added tert-butyl 7-(6-methyl-1H-indazol-3-yl)-3,4-dihydro-2H-quinoline-1-carboxylate (120 mg, 0.33 mmol, 1.0 equiv), DCM (2.5 mL), TFA (0.50 mL), the reaction was stirred at room temperature for 0.50 h. The reaction was concentrated directly. This was afforded 7-(6-methyl-1H-indazol-3-yl)-1,2,3,4-tetrahydroquinoline (120 mg, crude) as a yellow oil.

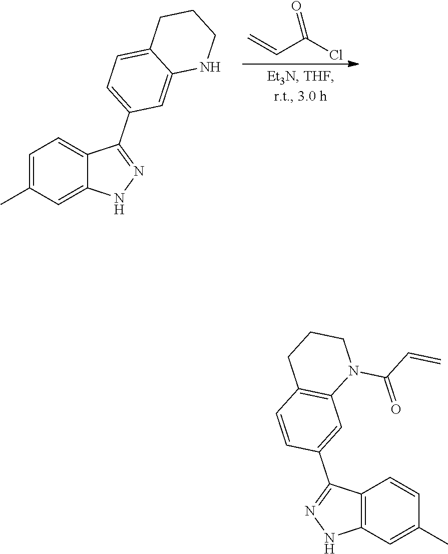

Into a 25 mL flask was added 7-(6-methyl-1H-indazol-3-yl)-1,2,3,4-tetrahydroquinoline (80.00 mg, 0.30 mmol, 1.0 equiv), THE (2.0 mL), Et₃N (93 mg, 0.92 mmol, 3.0 equiv), and acryloyl chloride (33 mg, 0.37 mmol, 1.2 equiv), in which the reaction was stirred at room temperature for 3.0 h. The reaction mixture was concentrated, and the residue was diluted in DMF (2 mL), and purified by reverse flash chromatography with the following conditions: column, silica gel; mobile phase, MeCN in water (0.05% in TFA), 10% to 50% gradient in 50 min; detector, UV 254 nm. The fractions were combined and concentrated and purified by Prep-HPLC using a XBridge Prep OBD C18 Column, 30*150 mm, Sum; Mobile Phase A: Water(10 mmol/L NH₄HCO₃+0.1% NH₃H₂O), Mobile Phase B: ACN; Flow rate: 60 mL/min; Gradient: 33% B to 63% B in 7 min, 63% B; wave length: 254 nm; RT1(min): 6. The fractions were combined and lyophilized directly, resulting in 1-[7-(6-methyl-1H-indazol-3-yl)-3,4-dihydro-2H-quinolin-1-yl]prop-2-en-1-one (19 mg, 19% yield) as a white solid.

LC/MS: mass calcd. For C₂₀H₁₉N₃O: 317.15, found: 318.15[M+H]⁺.

¹HNMR (300 MHz, DMSO-d6) δ: 13.05 (s, 1H), 7.88 (d, J=8.4 Hz, 1H), 7.72 (d, J=6.9 Hz, 2H), 7.33-7.36 (m, 2H), 7.04 (d, J=8.4 Hz, 1H), 6.66-6.75 (m, 1H), 6.27 (d, J=16.8 Hz, 1H), 5.78 (d, J=10.2 Hz, 1H), 3.81 (t, J=6.3 Hz, 2H), 2.78 (t, J=6.6 Hz, 2H), 2.45 (s, 3H), 1.90-1.98 (m, 2H).

### Example 3. Synthesis of 1-(6-{1H-pyrazolo[3,4-c]pyridin-3-yl}-2,3-dihydroindol-1-yl)prop-2-en-1-one (Compound 1)

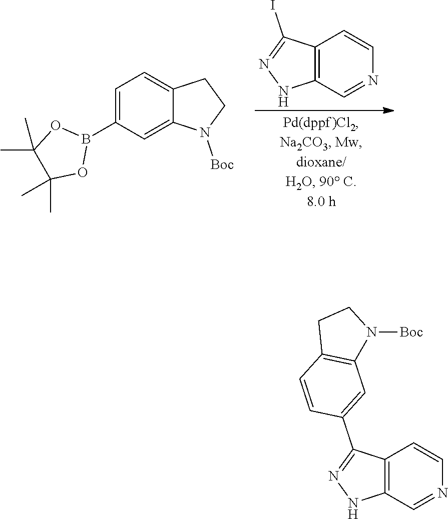 [SMILES: CC(C)(C)OC(=O)N1CCc2ccc(B3OC(C)(C)C(C)(C)O3)cc21 ; Ic1n[nH]c2cnccc12 ; CC(C)(C)OC(=O)N1CCc2ccc(-c3n[nH]c4cnccc34)cc21]

A solution of tert-butyl 6-(4,4,5,5-tetramethyl-1,3,2-dioxaborolan-2-yl)-2,3-dihydroindole-1-carboxylate (170 mg, 0.49 mmol, 1.0 equiv), Pd(dppf)Cl₂·CH₂Cl₂ (80 mg, 0.10 mmol, 0.20 equiv), Na₂CO₃ (260 mg, 2.5 mmol, 5.0 equiv) and 3-iodo-1H-pyrazolo[3,4-c]pyridine (120 mg, 0.49 mmol, 1.0 equiv) in 1,4-dioxane (4.0 mL) and H₂O (1.0 mL) was heated in a sealed tube in the microwave at 90° C. for 8.0 h under N₂ atmosphere. The solution was allowed to cool to room temperature and the reaction mixture was filtered, in which the filtrate was concentrated. The residue was purified by silica gel column chromatography and eluted with PE/EtOAc (1:1) to afford tert-butyl 6-{1H-pyrazolo[3,4-c]pyridin-3-yl}-2,3-dihydroindole-1-carboxylate (100 mg, 57%) as a brown solid.

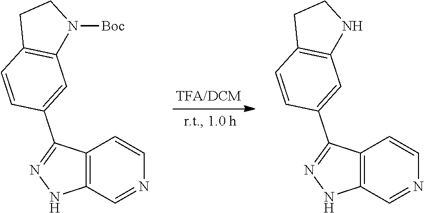 [SMILES: CC(C)(C)OC(=O)N1CCc2ccc(-c3n[nH]c4cnccc34)cc21 ; c1cc2c(-c3ccc4c(c3)NCC4)n[nH]c2cn1]

To a stirred solution of tert-butyl 6-{1H-pyrazolo[3,4-c]pyridin-3-yl}-2,3-dihydroindole-1-carboxylate (80 mg, 0.24 mmol, 1.0 equiv) in DCM (2.0 mL) was added TFA (0.50 mL) dropwise at room temperature. The resulting mixture was stirred for 1.0 h at room temperature and concentrated under vacuum to afford 6-{1H-pyrazolo[3,4-c]pyridin-3-yl}-2,3-dihydro-1H-indole (80 mg, crude) as a yellow oil.

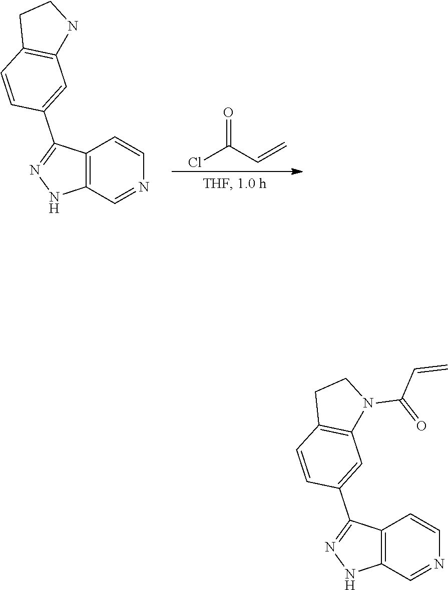

To a stirred solution of 6-{1H-pyrazolo[3,4-c]pyridin-3-yl}-2,3-dihydro-1H-indole (80 mg, 0.34 mmol, 1.0 equiv) in THE (2.0 mL) was added acryloyl chloride (37 mg, 0.41 mmol, 1.2 equiv) at 0° C. The resulting mixture was stirred for 1.0 h at room temperature and filtered in DMF (3.0 mL). The filtrate was purified by Prep-HPLC: Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 16% B to 46% B in 7 min, 46% B; wavelength: 254 nm; RT1(min): 6. The fractions were combined and lyophilized, resulting in 1-(6-{1H-pyrazolo[3,4-c]pyridin-3-yl}-2,3-dihydroindol-1-yl)prop-2-en-1-one (9.00 mg, 9.01%) as a light yellow solid.

LC/MS: mass calcd. For C₁₇H₁₄N₄O: 290.12, found: 291.10 [M+H]⁺.

¹H NMR (300 MHz, DMSO) δ: 13.33 (brs, 1H), 9.10 (s, 1H), 8.90 (s, 1H), 8.33 (d, J=5.7 Hz, 1H), 7.98 (d, J=5.7 Hz, 1H), 7.71 (d, J=7.8 Hz, 1H), 7.41 (d, J=7.8 Hz, 1H), 6.75-6.84 (m, 1H), 6.35 (d, J=16.5 Hz, 1H), 5.86 (d, J=10.2 Hz, 1H), 4.30 (t, J=8.4 Hz, 2H), 3.24 (t, J=8.4 Hz, 2H).

### Example 4. Synthesis of 1-(7-{1H-pyrazolo[3,4-c]pyridin-3-yl}-3,4-dihydro-2H-quinolin-1-yl)prop-2-en-1-one (Compound 10)

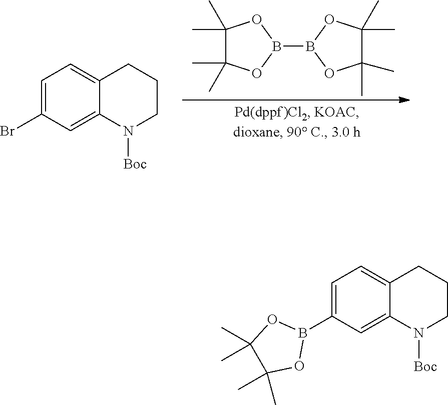

Into a 100 mL flask was added tert-butyl 7-bromo-3,4-dihydro-2H-quinoline-1-carboxylate (500 mg, 1.6 mmol, 1.0 equiv), bis(pinacolato)diboron (610 mg, 2.4 mmol, 1.5 equiv), KOAc (320 mg, 3.2 mmol, 2.0 equiv), Pd(dppf)Cl₂·CH₂Cl₂ (130 mg, 0.16 mmol, 0.10 equiv), and dioxane (15 mL). The reaction was stirred at 90° C. for 3.0 h under N₂ atmosphere. The reaction was quenched with water (30 mL) at room temperature and extracted with EtOAc (3×n30 mL). The combined organic layers were washed with brine (2×20 mL), and dried over anhydrous Na₂SO₄. The filtrate was then concentrated under reduced pressure, resulting in tert-butyl-7-(4,4,5,5-tetramethyl-1,3,2-dioxaborolan-2-yl)-3,4-dihydro-2H-quinoline-1-carboxylate (970 mg, crude) as a black solid.

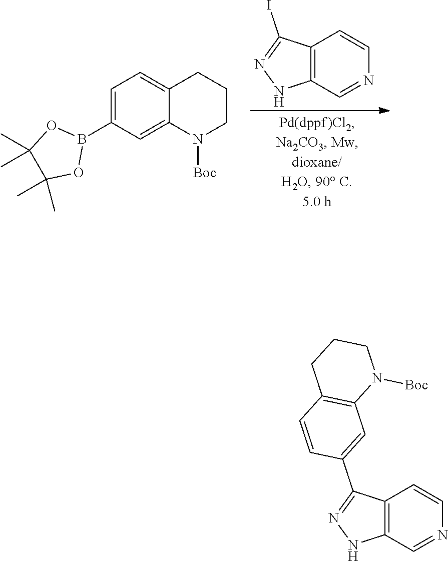

Into a 10 mL tube was added tert-butyl 7-(4,4,5,5-tetramethyl-1,3,2-dioxaborolan-2-yl)-3,4-dihydro-2H-quinoline-1-carboxylate (330 mg, 0.92 mmol, 1.5 equiv), 3-iodo-1H-pyrazolo[3,4-c]pyridine (150 mg, 0.61 mmol, 1.0 equiv), Na₂CO₃ (200 mg, 1.9 mmol, 3.0 equiv), Pd(dppf)Cl₂·CH₂Cl₂ (55 mg, 0.06 mmol, 0.10 equiv), dioxane (4.0 mL), and H₂O (1.0 mL). The reaction was stirred at 90° C. for 5.0 h and under N₂ atmosphere by a microwave reactor. The resulting mixture was filtered, and the filter cake was washed with MeOH (3×10 mL). The filtrate was concentrated under reduced pressure, and the residue was purified by silica gel column chromatography, and eluted with CH₂Cl₂/MeOH (15:1) to afford tert-butyl 7-{1H-pyrazolo[3,4-c]pyridin-3-yl}-3,4-dihydro-2H-quinoline-1-carboxylate (170 mg, 55% yield) as a brown solid.

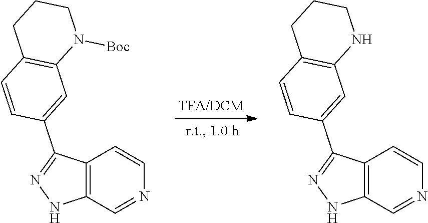 [SMILES: CC(C)(C)OC(=O)N1CCCc2ccc(-c3n[nH]c4cnccc34)cc21 ; c1cc2c(-c3ccc4c(c3)NCCC4)n[nH]c2cn1]

Into a 50 mL flask was added tert-butyl 7-{1H-pyrazolo[3,4-c]pyridin-3-yl}-3,4-dihydro-2H-quinoline-1-carboxylate (150 mg, 0.43 mmol, 1.0 equiv), DCM (2.5 mL), TFA (0.5 mL), the reaction was stirred at room temperature for 1.0 h. The reaction was concentrated, resulting in 7-{1H-pyrazolo[3,4-c]pyridin-3-yl}-1,2,3,4-tetrahydroquinoline (150 mg, crude) as a yellow oil.

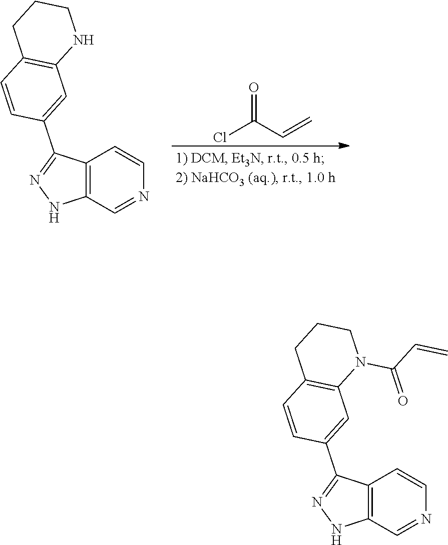

Into a 25 mL flask was added 7-{1H-pyrazolo[3,4-c]pyridin-3-yl}-1,2,3,4-tetrahydroquinoline (100 mg, 0.40 mmol, 1.0 equiv), DCM (5.0 mL), Et₃N (120 mg, 1.2 mmol, 3.0 equiv), and acryloyl chloride (44 mg, 0.49 mmol, 1.2 equiv), in which the reaction was stirred at room temperature for 0.5 h. Then 1 mL NaHCO₃ (aq) was added and stirred at room temperature for 1.0 h and concentrated directly. The residue was dissolved in DMF (2 mL) and purified by Prep-HPLC: Column: Xselect CSH C18 OBD Column 30*150 mm Sum; Mobile Phase A: Water(0.05% TFA), Mobile Phase B: ACN; Flow rate: 60 mL/min; Gradient: 10% B to 33% B in 7 min, 33% B; wavelength: 254 nm; RT1(min): 5.45. The fractions were combined and lyophilized directly, resulting in 1-(7-{1H-pyrazolo[3,4-c]pyridin-3-yl}-3,4-dihydro-2H-quinolin-1-yl)prop-2-en-1-one (8.6 mg, 6.8%) as a white solid.

LC/MS: mass calcd. For C₁₈H₁₆N₄O: 304.13, found: 305.15[M+H]⁺.

¹HNMR (300 MHz, DMSO-d6) δ: 14.17 (s, 1H), 9.29 (s, 1H), 8.38 (d, J=5.7 Hz, 1H), 8.18 (s, 1H), 7.79-7.86 (m, 2H), 7.41 (d, J=8.1 Hz, 1H), 6.69-6.78 (m, 1H), 6.30 (d, J=13.8 Hz, 1H), 5.80 (d, J=10.2 Hz, 1H), 3.82 (t, J=6.6 Hz, 2H), 2.82 (t, J=6.6 Hz, 2H), 1.91-2.00 (m, 2H).

### Example 5. Synthesis of 1-(6-{1H-pyrazolo[4,3-c]pyridin-3-yl}-2,3-dihydroindol-1-yl)prop-2-en-1-one (Compound 3)

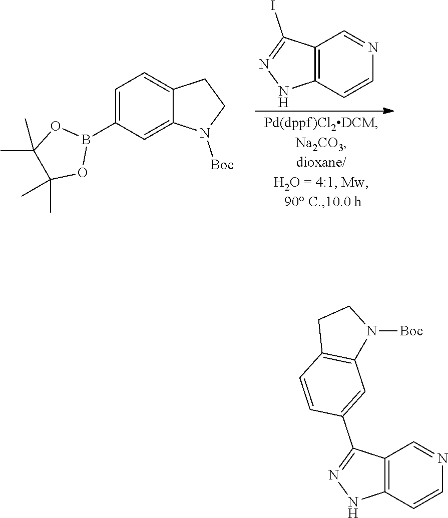 [SMILES: CC(C)(C)OC(=O)N1CCc2ccc(B3OC(C)(C)C(C)(C)O3)cc21 ; Ic1n[nH]c2ccncc12 ; CC(C)(C)OC(=O)N1CCc2ccc(-c3n[nH]c4ccncc34)cc21]

To a solution of 3-iodo-1H-pyrazolo[4,3-c]pyridine (100 mg, 0.41 mmol, 1.0 equiv) and tert-butyl 6-(4,4,5,5-tetramethyl-1,3,2-dioxaborolan-2-yl)-2,3-dihydroindole-1-carboxylate (280 mg, 0.82 mmol, 2.0 equiv) in dioxane (2.0 mL) and H₂O (0.50 mL), Pd(dppf)Cl₂·CH₂Cl₂ (66 mg, 0.08 mmol, 0.20 equiv) and Na₂CO₃ (220 mg, 2.0 mmol, 5.0 equiv) was added. The final reaction mixture was irradiated with microwave radiation for 10 h at 90° C. The resulting mixture was filtered, and the filter cake was washed with EtOAc (3×10 mL). 10 mL of water was added and extracted with EtOAc (3×10 mL). The combined organic layers were washed with brine (1×10 mL), dried over anhydrous Na₂SO₄. The filtrate was concentrated under reduced pressure, and the residue was purified by silica gel column chromatography, eluted with PE/EA (0-80% EA) to afford tert-butyl 6-{1H-pyrazolo[4,3-c]pyridin-3-yl}-2,3-dihydroindole-1-carboxylate (60 mg, 39%) as an off-white solid.

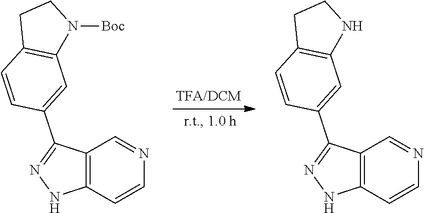 [SMILES: CC(C)(C)OC(=O)N1CCc2ccc(-c3n[nH]c4ccncc34)cc21 ; c1cc2[nH]nc(-c3ccc4c(c3)NCC4)c2cn1]

To a stirred solution of tert-butyl 6-{1H-pyrazolo[4,3-c]pyridin-3-yl}-2,3-dihydroindole-1-carboxylate (50 mg, 0.15 mmol, 1.0 equiv) in DCM (1.0 mL) was added TFA (0.25 mL) dropwise at 0° C. The resulting mixture was stirred for 1.0 h at room temperature. After reaction, the resulting mixture was concentrated under vacuum, resulting in 6-{1H-pyrazolo[4,3-c]pyridin-3-yl}-2,3-dihydro-1H-indole (50 mg, crude) as a brown yellow oil.

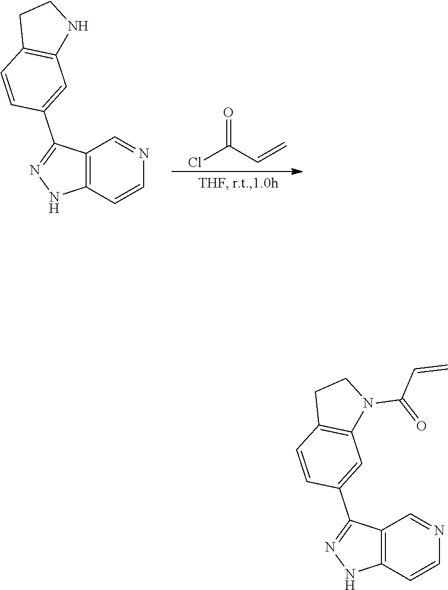

To a stirred solution of 6-{1H-pyrazolo[4,3-c]pyridin-3-yl}-2,3-dihydro-1H-indole (50 mg, 0.21 mmol, 1.0 equiv) in THE (1.0 mL) was added acryloyl chloride (29 mg, 0.32 mmol, 1.5 equiv) dropwise at 0° C. The resulting mixture was stirred for 1.0 h at room temperature, and concentrated under vacuum. The residue was dissolved in DMF (1.0 mL), in which the solid was filtered out. The filtrate was purified by Prep-HPLC directly with the following conditions: Column: XBridge Prep OBD C18 Column, 19*250 mm, 5 μm; Mobile Phase A: Water (0.05% TFA), Mobile Phase B: ACN; Flow rate: 25 mL/min; Gradient: 13% B to 31% B in 10 min, 31% B; wavelength: 254 nm; RT1(min): 8.18. The fractions were combined and lyophilized directly. This resulted in 1-(6-{1H-pyrazolo[4,3-c]pyridin-3-yl}-2,3-dihydroindol-1-yl)prop-2-en-1-one (1.9 mg, 3.1%) as a white solid.

LC/MS: mass calcd. For C₁₇H₁₄N₄O: 290.12, found: 291.15.15 [M+H]⁺.

¹H NMR (300 MHz, DMSO-d6) δ: 14.48 (brs, 1H), 9.66 (s, 1H), 8.90 (s, 1H), 8.54 (d, J=6.6 Hz, 1H), 8.02 (d, J=6.6 Hz, 1H), 7.78 (d, J=8.4 Hz, 1H), 7.46 (d, J=8.4 Hz, 1H), 6.75-6.85 (m, 1H), 6.35 (d, J=16.5 Hz, 1H), 5.87 (d, J=10.2 Hz, 1H), 4.31 (t, J=8.4 Hz, 2H), 3.26 (t, J=8.4 Hz, 2H).

### Example 6. Synthesis of 1-(7-{1H-pyrazolo[4,3-c]pyridin-3-yl}-3,4-dihydro-2H-quinolin-1-yl)prop-2-en-1-one (Compound 11)

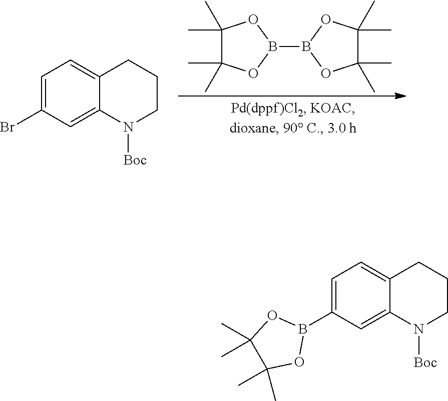

Into a 100 mL flask was added tert-butyl 7-bromo-3,4-dihydro-2H-quinoline-1-carboxylate (500 mg, 1.6 mmol, 1.0 equiv), bis(pinacolato)diboron (610 mg, 2.4 mmol, 1.5 equiv), KOAc (320 mg, 3.2 mmol, 2.0 equiv), Pd(dppf)Cl₂·CH₂Cl₂ (130 mg, 0.16 mmol, 0.10 equiv), dioxane (15 mL). The reaction was stirred at 90° C. for 3.0 h under N₂ atmosphere, and quenched with water (30 mL) at room temperature. The resulting mixture was extracted with EtOAc (3×30 mL), and the organic layers were combined and washed with brine (2×20 mL), dried over anhydrous Na₂SO₄. After filtration, the filtrate was concentrated under reduced pressure, resulting in tert-butyl-7-(4,4,5,5-tetramethyl-1,3,2-dioxaborolan-2-yl)-3,4-dihydro-2H-quinoline-1-carboxylate (970 mg, crude) as a black solid.

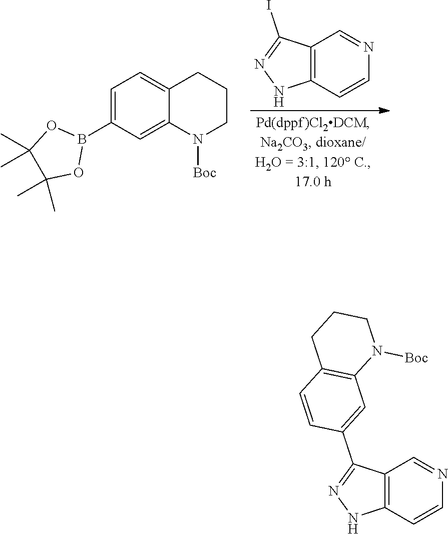

To a solution of 3-iodo-1H-pyrazolo[4,3-c]pyridine (150 mg, 0.61 mmol, 1.0 equiv) and tert-butyl-7-(4,4,5,5-tetramethyl-1,3,2-dioxaborolan-2-yl)-3,4-dihydro-2H-quinoline-1-carboxylate (220 mg, 0.61 mmol, 1.0 equiv) in dioxane (3.0 mL) and H₂O (1.0 mL) were added Pd(dppf)Cl₂·CH₂Cl₂ (100 mg, 0.12 mmol, 0.20 equiv) and Na₂CO₃ (320 mg, 3.1 mmol, 5.0 equiv) under N₂. The final reaction mixture was stirred for 17 h at 120° C., and the resulting mixture was filtered, in which the filter cake was washed with EtOAc (3×10 mL). The filtrate was extracted with EtOAc (30 mL) and water (30 mL). The combined organic layers were washed with brine (1×10 mL), and dried over anhydrous Na₂SO₄. After filtration, the filtrate was concentrated under reduced pressure, and the residue was purified by silica gel column chromatography, eluted with PE/EA (1:8) to afford tert-butyl 7-{1H-pyrazolo[4,3-c]pyridin-3-yl}-3,4-dihydro-2H-quinoline-1-carboxylate (100 mg, 42%) as off-white solid.

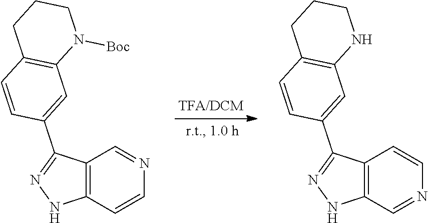 [SMILES: CC(C)(C)OC(=O)N1CCCc2ccc(-c3n[nH]c4ccncc34)cc21 ; c1cc2c(-c3ccc4c(c3)NCCC4)n[nH]c2cn1]

To a stirred solution of tert-butyl 7-{1H-pyrazolo[4,3-c]pyridin-3-yl}-3,4-dihydro-2H-quinoline-1-carboxylate (90 mg, 0.26 mmol, 1.0 equiv) in DCM (2.0 mL) was added TFA (0.50 mL) dropwise at 0° C. The resulting mixture was stirred for 1.0 h at room temperature, and the resulting mixture was concentrated under vacuum. The product 6-{1H-pyrazolo[4,3-c]pyridin-3-yl}-2,3-dihydro-1H-indole (90 mg, crude) was obtained as a brown yellow oil.

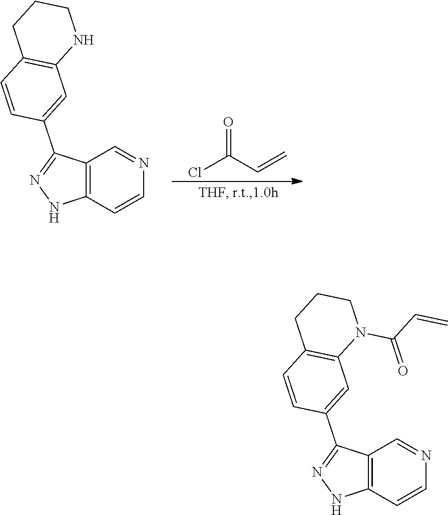

To a stirred solution of 7-{1H-pyrazolo[4,3-c]pyridin-3-yl}-1,2,3,4-tetrahydroquinoline (70 mg, 0.28 mmol, 1.0 equiv) in THE (1.0 mL) was added acryloyl chloride (38 mg, 0.42 mmol, 1.5 equiv) dropwise at 0° C. The resulting mixture was stirred for 1.0 h at room temperature and concentrated under vacuum. The residue was dissolved in DMF (1 mL) and purified by Prep-HPLC directly: Column: XBridge Prep Phenyl OBD Column, 19*250 mm, 5 μm; Mobile Phase A: Water (0.05% TFA), Mobile Phase B: ACN; Flow rate: 25 mL/min; Gradient: 26% B to 56% B in 7 min, 56% B; wavelength: 254 nm; RT1(min): 6. The fractions were combined and lyophilized directly, resulting in 1-(7-{1H-pyrazolo[4,3-c]pyridin-3-yl}-3,4-dihydro-2H-quinolin-1-yl)prop-2-en-1-one (11 mg, 12%) as a yellow solid.

LC/MS: mass calcd. For C₁₈H₁₆N₄O: 304.13, found: 305.15 [M+H]⁺.

¹H NMR (300 MHz, DMSO-d6) δ: 14.59 (s, 1H), 9.67 (s, 1H), 8.57 (d, J=6.6 Hz, 1H), 8.07 (d, J=6.6 Hz, 1H), 7.93 (s, 1H), 7.86 (d, J=8.1 Hz, 1H), 7.45 (d, J=7.8 Hz, 1H), 6.70-6.78 (m, 1H), 6.29 (d, J=16.8 Hz, 1H), 5.80 (d, J=10.2 Hz, 1H), 3.83 (t, J=6.6 Hz, 2H), 2.83 (t, J=6.3 Hz, 2H), 1.92-2.01 (m, 2H).

¹H NMR (300 MHz, DMSO-d6+D₂O) δ: 9.61 (s, 1H), 8.51 (d, J=6.9 Hz, 1H), 8.07 (d, J=6.9 Hz, 1H), 7.90 (s, 1H), 7.82 (d, J=7.8 Hz, 1H), 7.45 (d, J=8.1 Hz, 1H), 6.65-6.74 (m, 1H), 6.28 (d, J=16.8 Hz, 1H), 5.80 (d, J=10.2 Hz, 1H), 3.83 (t, J=6.6 Hz, 1H), 2.81 (t, J=6.3 Hz, 2H), 1.89-1.98 (m, 2H).

### Example 7. Synthesis of 1-[4-(1H-indazol-3-yl)-2,3-dihydroindol-1-yl]prop-2-en-1-one (Compound 2)

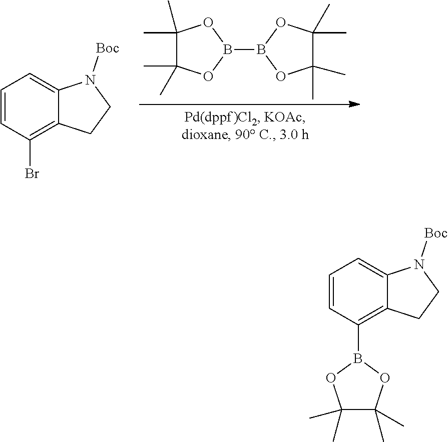

To a stirred solution of tert-butyl 4-bromo-2,3-dihydroindole-1-carboxylate (300 mg, 1.0 mmol, 1.0 equiv) in 1,4-dioxane (5.0 mL), bis(pinacolato)diboron (380 mg, 1.5 mmol, 1.5 equiv), Pd(dppf)Cl₂·CH₂Cl₂ (82 mg, 0.10 mmol, 0.10 equiv) and KOAc (197 mg, 2.0 mmol, 2.0 equiv) was added at room temperature under nitrogen atmosphere. The resulting mixture was stirred for 3.0 h at 90° C. under nitrogen atmosphere. The mixture was allowed to cool down to room temperature and quenched by the addition of water (10 mL) at 0° C. The resulting mixture was extracted with EtOAc (3×10 mL). The combined organic layers were washed with water (2×5 mL), and dried over anhydrous Na₂SO₄. The filtrate was concentrated under reduced pressure, resulting in tert-butyl 4-(4,4,5,5-tetramethyl-1,3,2-dioxaborolan-2-yl)-2,3-dihydroindole-1-carboxylate (300 mg, 86%) as a dark yellow oil.

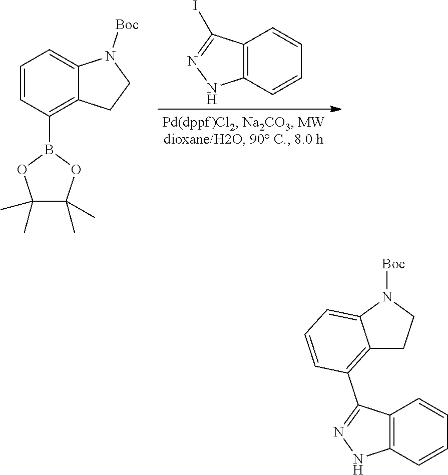 [SMILES: CC(C)(C)OC(=O)N1CCc2c(B3OC(C)(C)C(C)(C)O3)cccc21 ; Ic1n[nH]c2ccccc12 ; CC(C)(C)OC(=O)N1CCc2c(-c3n[nH]c4ccccc34)cccc21]

A solution of tert-butyl 4-(4,4,5,5-tetramethyl-1,3,2-dioxaborolan-2-yl)-2,3-dihydroindole-1-carboxylate (170 mg, 0.49 mmol, 1.0 equiv), 3-iodo-1H-indazole (120 mg, 0.49 mmol, 1.0 equiv), Pd(dppf)Cl₂·CH₂Cl₂ (80 mg, 0.10 mmol, 0.20 equiv) and Na₂CO₃ (261 mg, 2.5 mmol, 5.0 equiv) in 1,4-dioxane (4.0 mL) and H₂O (1.0 mL) were heated in a sealed tube in the microwave at 90° C. for 8.0 hour under N₂ atmosphere. The solution was allowed to cool to room temperature. The reaction mixture was filtered and the filtrate was concentrated. The residue was purified by silica gel column chromatography, eluted with PE/EtOAc (1:1) to afford tert-butyl 4-(1H-indazol-3-yl)-2,3-dihydroindole-1-carboxylate (90 mg, 50%) as a light pink solid.

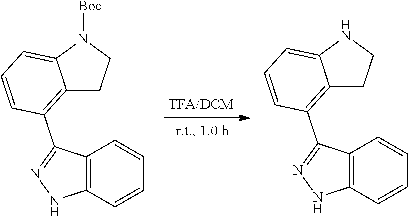 [SMILES: CC(C)(C)OC(=O)N1CCc2c(-c3n[nH]c4ccccc34)cccc21 ; c1cc2c(c(-c3n[nH]c4ccccc34)c1)CCN2]

To a stirred solution of tert-butyl 4-(1H-indazol-3-yl)-2,3-dihydroindole-1-carboxylate (90 mg, 0.27 mmol, 1.0 equiv) in DCM (2.0 mL) was added TFA (0.50 mL) dropwise at room temperature. The resulting mixture was stirred for 1.0 h at room temperature. The resulting mixture was concentrated under vacuum. 3-(2,3-dihydro-1H-indol-4-yl)-1H-indazole (90 mg, crude) was obtained as a yellow oil.

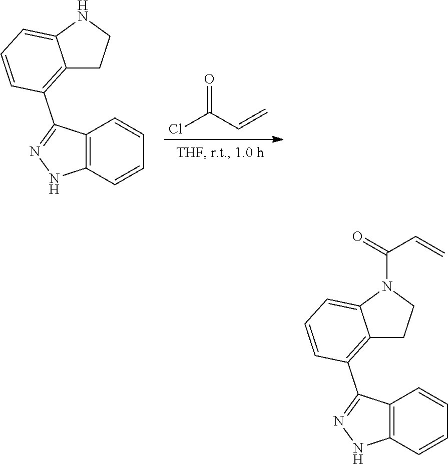

To a stirred solution of 3-(2,3-dihydro-1H-indol-4-yl)-1H-indazole (80 mg, 0.34 mmol, 1.0 equiv) in THE (1.5 mL) was added acryloyl chloride (37 mg, 0.41 mmol, 1.2 equiv) at 0° C. The resulting mixture was stirred for 1.0 h at room temperature, and subsequently filtered. The filtration in DMF (3.0 mL) was purified by Prep-HPLC: Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 25% B to 55% B in 7 min, 55% B; wavelength: 254 nm; RT1(min): 6.87. The fractions were combined and lyophilized, in which 1-[4-(1H-indazol-3-yl)-2,3-dihydroindol-1-yl]prop-2-en-1-one (25 mg, 26%) was obtained as white solid.

LC/MS: mass calcd. for C₁₈H₁₅N₃O: 289.12, found: 290.15 [M+H]⁺.

¹H NMR (300 MHz, DMSO) δ: 13.31 (s, 1H), 8.25 (d, J=6.9 Hz, 1H), 7.93 (d, J=8.1 Hz, 1H), 7.52-7.62 (m, 2H), 7.34-7.44 (m, 2H), 7.20 (t, J=7.8 Hz, 1H), 6.74-6.83 (m, 1H), 6.33 (d, J=16.5 Hz, 1H), 5.83 (d, J=10.5 Hz, 1H), 4.28 (t, J=8.1 Hz, 2H), 3.42 (t, J=8.1 Hz, 2H).

### Example 8. Synthesis of 1-[6-(1H-indazol-3-yl)-2,3-dihydro-1,4-benzoxazin-4-yl]prop-2-en-1-one (Compound 13)

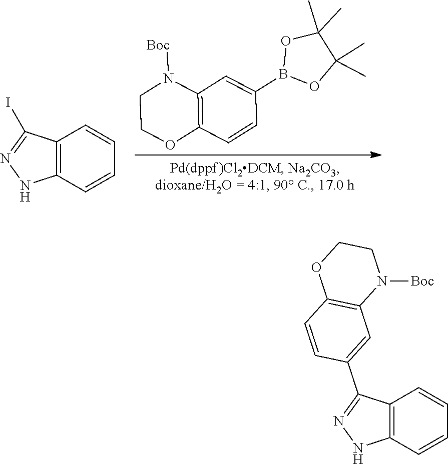

To a solution of 3-iodo-1H-indazole (120 mg, 0.49 mmol, 1.0 equiv) in 1,4-dioxane (4.0 mL) and H₂O (1.0 mL), tert-butyl 6-(4,4,5,5-tetramethyl-1,3,2-dioxaborolan-2-yl)-2,3-dihydro-1,4-benzoxazine-4-carboxylate (210 mg, 0.59 mmol, 1.2 equiv), Pd(dppf)Cl₂·CH₂Cl₂ (80 mg, 0.10 mmol, 0.20 equiv) and Na₂CO₃ (261 mg, 2.5 mmol, 5.0 equiv) was added and stirred at 90° C. for 17 h under N₂ atmosphere. The reaction mixture was filtered, and the filtrate was concentrated. The residue was purified by silica gel column chromatography, and eluted with PE/EtOAc (1:1) to afford tert-butyl 6-(1H-indazol-3-yl)-2,3-dihydro-1,4-benzoxazine-4-carboxylate (120 mg, 63%) as a white solid.

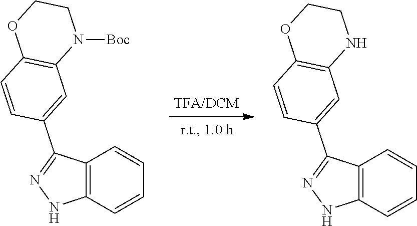 [SMILES: CC(C)(C)OC(=O)N1CCOc2ccc(-c3n[nH]c4ccccc34)cc21 ; c1ccc2c(-c3ccc4c(c3)NCCO4)n[nH]c2c1]

To a stirred solution of tert-butyl 6-(1H-indazol-3-yl)-2,3-dihydro-1,4-benzoxazine-4-carboxylate (120 mg, 0.34 mmol, 1.0 equiv) in DCM (2.0 mL), TFA (0.50 mL) was added dropwise at room temperature. The resulting mixture was stirred for 1.0 h at room temperature. The resulting mixture was concentrated under vacuum and 6-(1H-indazol-3-yl)-3,4-dihydro-2H-1,4-benzoxazine (120 mg, crude) was obtained as a yellow oil.

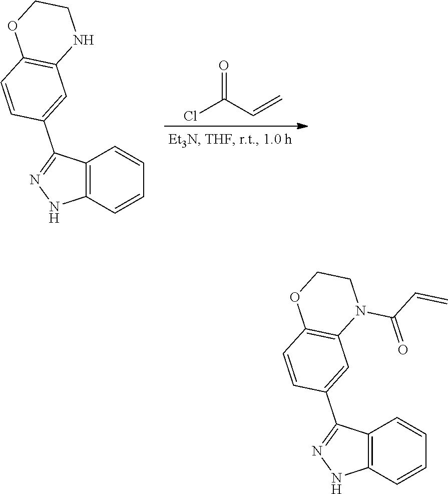

To a stirred solution of 6-(1H-indazol-3-yl)-3,4-dihydro-2H-1,4-benzoxazine (100 mg, 0.40 mmol, 1.0 equiv) in THE (2.0 mL), acryloyl chloride (43 mg, 0.48 mmol, 1.2 equiv) at 0° C. was added. The resulting mixture was stirred for 1.0 h at room temperature. The reaction mixture was filtered and the filtration in DMF (3.0 mL) was purified by Prep-HPLC: Column: XBridge Prep OBD C18 Column, 30*150 mm, 5 μm; Mobile Phase A: Water (0.05% TFA), Mobile Phase B: ACN; Flow rate: 60 mL/min; Gradient: 25% B to 55% B in 9 min, 55% B; wavelength: 254 nm; RT1(min): 7. The fractions were combined and lyophilized, in which 1-[6-(1H-indazol-3-yl)-2,3-dihydro-1,4-benzoxazin-4-yl]prop-2-en-1-one (25 mg, 20%) was obtained as a white solid.

LC/MS: mass calcd. For C₁₈H₁₅N₃O₂: 305.12, found: 306.10 [M+H]⁺.

¹H NMR (300 MHz, DMSO) δ: 13.10 (s, 1H), 8.10 (s, 1H), 8.00 (d, J=8.1 Hz, 1H), 7.70 (d, J=8.4 Hz, 1H), 7.57 (d, J=8.4 Hz, 1H), 7.39 (t, J=7.2 Hz, 1H), 7.20 (t, J=7.2 Hz, 1H), 7.06 (d, J=8.7 Hz, 1H), 6.86-6.95 (m, 1H), 6.33 (d, J=14.7 Hz, 1H), 5.88 (d, J=10.2 Hz, 1H), 4.36 (t, J=4.5 Hz, 2H), 4.01 (t, J=4.5 Hz, 2H).

### Example 9. Synthesis of (2E)-1-[6-(1H-indazol-3-yl)-2,3-dihydroindol-1-yl]but-2-en-1-one (Compound 9)

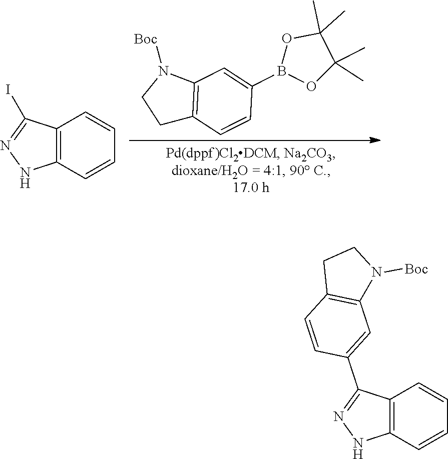

To a solution of 3-iodo-1H-indazole (2.0 g, 8.2 mmol, 1.0 equiv) in 1,4-dioxane (16 mL) and H₂O (4.0 mL), tert-butyl 6-(4,4,5,5-tetramethyl-1,3,2-dioxaborolan-2-yl)-2,3-dihydroindole-1-carboxylate (2.8 g, 8.2 mmol, 1.0 equiv), Pd(dppf)Cl₂·CH₂Cl₂ (1.3 g, 1.7 mmol, 0.20 equiv) and Na₂CO₃ (4.3 g, 41 mmol, 5.0 equiv) was added, with stirring at 90° C. overnight under N₂ atmosphere. The reaction mixture was filtered, and the filtrate was concentrated. The residue was purified by silica gel column chromatography, and eluted with PE/EtOAc(1:1) to afford tert-butyl 6-(1H-indazol-3-yl)-2,3-dihydroindole-1-carboxylate (2.1 g, 62%) as an orange solid.

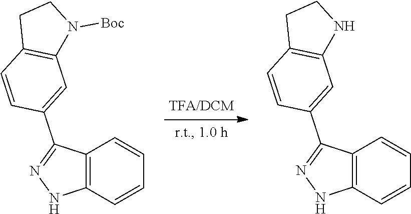 [SMILES: CC(C)(C)OC(=O)N1CCc2ccc(-c3n[nH]c4ccccc34)cc21 ; c1ccc2c(-c3ccc4c(c3)NCC4)n[nH]c2c1]

To a stirred solution of tert-butyl 6-(1H-indazol-3-yl)-2,3-dihydroindole-1-carboxylate (1.0 g, 3.0 mmol, 1.0 equiv) in DCM (10 mL) was added TFA (2.5 mL) dropwise at room temperature. The resulting mixture was stirred for 1.0 h at room temperature. The resulting mixture was concentrated under vacuum and 3-(2,3-dihydro-1H-indol-6-yl)-1H-indazole (1.0 g, crude) was obtained as a brown solid.

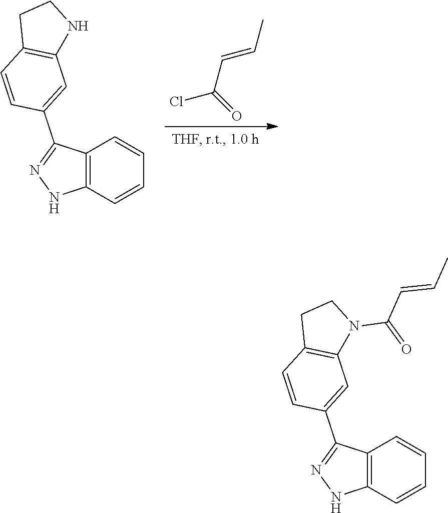

To a stirred solution of 3-(2,3-dihydro-1H-indol-6-yl)-1H-indazole (100 mg, 0.42 mmol, 1.0 equiv) in THE (10 mL), 2-butenoyl chloride (47 mg, 0.44 mmol, 1.1 equiv) was added dropwise at 0° C. with stirring for 1.0 h at room temperature. After reaction, the resulting mixture was concentrated under reduced pressure. The mixture was purified by reverse phase column directly with the following conditions: Column: XBridge Shield RP18 OBD Column, 30*150 mm, 5 μm; Mobile Phase A: Water(10 mmol/L NH₄HCO₃), Mobile Phase B: ACN; Flow rate: 60 mL/min; Gradient: 30% B to 46% B in 10 min, 46% B; wavelength: 254 nm; RT1(min): 9.68; Number Of Runs: 0. The fractions were combined and concentrated, resulting in (2E)-1-[6-(1H-indazol-3-yl)-2,3-dihydroindol-1-yl]but-2-en-1-one (23.80 mg, 18.%) as a white solid.

LC/MS: mass calcd. For C₁₉H₁₇N₃O: 303.14, found: 304.05 [M+H]⁺.

¹H NMR (300 MHz, DMSO-d₆) δ: 13.20 (s, 1H), 8.86 (s, 1H), 8.02 (d, J=8.4 Hz, 1H), 7.58-7.66 (m, 2H), 7.35-7.46 (m, 2H), 7.19-7.24 (m, 1H), 6.86-6.98 (m, 1H), 6.49 (d, J=15.0 Hz, 1H), 4.25 (t, J=8.4 Hz, 2H), 3.21 (t, J=8.4 Hz, 2H), 1.94 (d, J=6.8 Hz, 3H).

### Example 10. Synthesis of 2-fluoro-1-[6-(1H-indazol-3-yl)-2,3-dihydroindol-1-yl]prop-2-en-1-one (Compound 8)

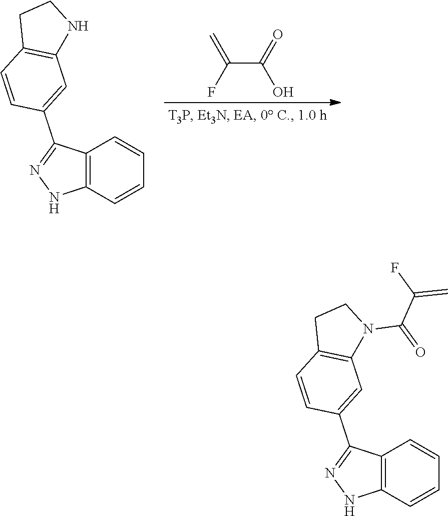

To a stirred solution of 3-(2,3-dihydro-1H-indol-6-yl)-1H-indazole (100 mg, 0.43 mmol, 1.0 equiv) and 2-fluoroacrylic acid (120 mg, 1.30 mmol, 3.0 equiv) in EA (1.0 mL), TEA (350 μL, 2.6 mmol, 6.0 equiv) and T₃P (340 μL, 1.3 mmol, 3.0 equiv) was added in dropwise at 0° C. The resulting mixture was stirred for 1.0 h at room temperature, and quenched by H₂O (10 mL). The mixture was extracted with EtOAc (3×10 mL) and the organic layers were combined and washed with brine (1×5 mL), dried over anhydrous Na₂SO₄. After filtration, the filtrate was concentrated under reduced pressure and the residue was dissolved in DMF (2 mL) and purified by Prep-HPLC: Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 33% B to 60% B in 7 min, 60% B; wavelength: 254 nm; RT1(min): 6.53. The fractions were combined and lyophilized directly. This resulted in 2-fluoro-1-[6-(1H-indazol-3-yl)-2,3-dihydroindol-1-yl]prop-2-en-1-one (41 mg, 31%) as off-white solid.

LC/MS: mass calcd. For C₁₈H₁₄FN₃O: 307.11, found: 308.15 [M+H]⁺.

¹H NMR (300 MHz, DMSO-d6) δ: 13.23 (s, 1H), 8.67 (s, 1H), 8.03 (d, J=8.1 Hz, 1H), 7.74 (d, J=7.5 Hz, 1H), 7.61 (d, J=8.4 Hz, 1H), 7.39-7.46 (m, 2H), 7.23 (t, J=7.5 Hz, 1H), 5.44-5.66 (m, 2H), 4.29-4.35 (m, 2H), 3.23 (t, J=7.8 Hz, 2H).

### Example 11. Synthesis of (2E)-4-(dimethylamino)-1-[6-(1H-indazol-3-yl)-2,3-dihydroindol-1-yl]but-2-en-1-one (Compound 6)

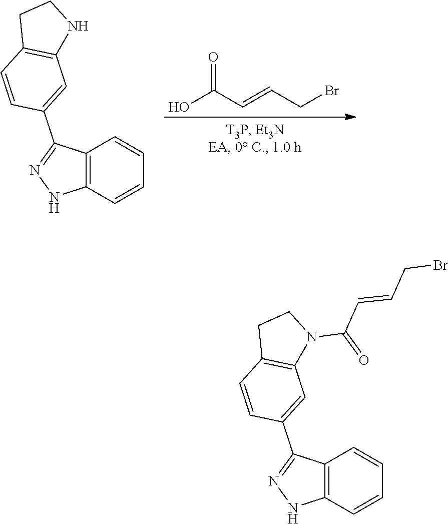 [SMILES: c1ccc2c(-c3ccc4c(c3)NCC4)n[nH]c2c1 ; O=C(O)/C=C/CBr ; O=C(/C=C/CBr)N1CCc2ccc(-c3n[nH]c4ccccc34)cc21]

To a solution of (2E)-4-bromobut-2-enoic acid (110 mg, 0.62 mmol, 0.75 equiv), 3-(2,3-dihydro-1H-indol-6-yl)-1H-indazole (200 mg, 0.84 mmol, 1.0 equiv) and Et₃N (520 mg, 5.1 mmol, 6.0 equiv) in EA (10 mL), T₃P (810 mg, 2.5 mmol, 3.0 equiv) was added at 0° C. The mixture was stirred at 0° C. for 1.0 h, and subsequently poured into ice water (10 mL) and extracted by EA (3×10 mL). The combined organic layer was concentrated and the crude (150 mg) was used in the next step.

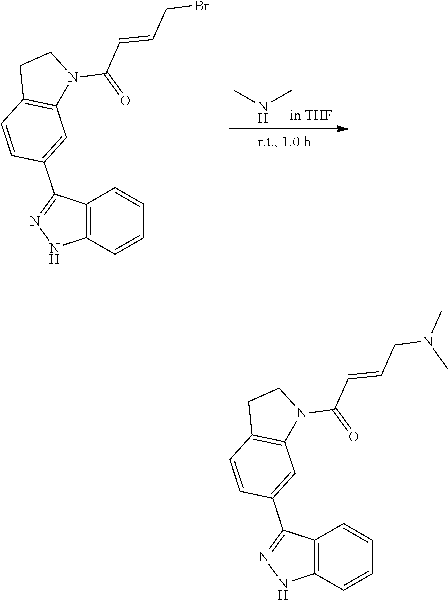

To a stirred solution of (2E)-4-bromo-1-[6-(1H-indazol-3-yl)-2,3-dihydroindol-1-yl]but-2-en-1-one (150 mg, 0.39 mmol, 1.0 equiv) in THE (2.0 mL), dimethylamine (2M in THF) (1.0 mL, 2.0 mmol, 5.0 equiv) was added dropwise at 0° C. The resulting mixture was stirred for 1.0 h at room temperature.

After reaction, the resulting mixture was concentrated under reduced pressure. The mixture was purified by reverse phase column directly with the following conditions: Column: Xselect CSH C18 OBD Column 30*150 mm 5 μm; Mobile Phase A: Water (0.05% TFA), Mobile Phase B: ACN; Flow rate: 60 mL/min; Gradient: 5% B to 35% B in 10 min, 35% B; wavelength: 254 nm; RT1(min): 9.20; Number Of Runs: 0. The fraction were combined and concentrated, resulting in (2E)-4-(dimethylamino)-1-[6-(1H-indazol-3-yl)-2,3-dihydroindol-1-yl]but-2-en-1-one (27 mg, 20%) as a white solid.

LC/MS: mass calcd. For C₂₁H₂₂N₄O: 346.18, found: 347.10 [M+H]⁺.

¹H NMR (400 MHz, DMSO-d₆) δ: 8.87 (s, 1H), 8.03 (d, J=8.8 Hz, 1H), 7.70 (d, J=7.6 Hz, 1H), 7.61 (d, J=8.4 Hz, 1H), 7.41-7.45 (m, 2H), 7.22-7.26 (m, 1H), 6.80-6.92 (m, 2H), 4.31 (t, J=8.4 Hz, 2H), 3.99 (d, J=6.4 Hz, 2H), 3.27 (t, J=8.4 Hz, 2H), 2.84 (s, 6H).

### Example 12. Synthesis of (2E)-4-[6-(1H-indazol-3-yl)-2,3-dihydroindol-1-yl]-4-oxobut-2-enenitrile (Compound 5)

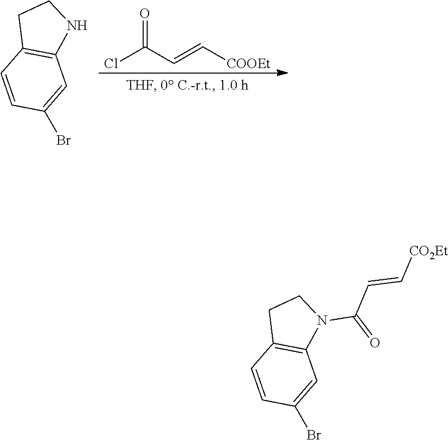 [SMILES: Brc1ccc2c(c1)NCC2 ; CCOC(=O)/C=C/C(=O)Cl ; CCOC(=O)/C=C/C(=O)N1CCc2ccc(Br)cc21]

To a stirred solution of 6-bromo-2,3-dihydro-1H-indole (1.0 g, 5.10 mmol, 1.0 equiv) in THE (10 mL), ethyl (2E)-4-chloro-4-oxobut-2-enoate (0.98 g, 6.1 mmol, 1.2 equiv) was added at 0° C. The resulting mixture was stirred for 1.0 h at room temperature and the residue was purified by silica gel column chromatography, and eluted with PE/EtOAc (3:1) to afford ethyl (2E)-4-(6-bromo-2,3-dihydroindol-1-yl)-4-oxobut-2-enoate (1.50 g, 91%) to yield a yellow green solid.

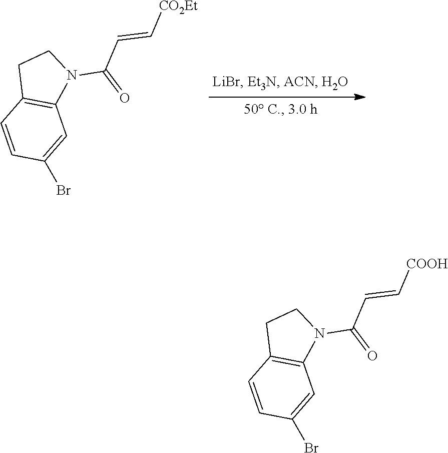 [SMILES: CCOC(=O)/C=C/C(=O)N1CCc2ccc(Br)cc21 ; O=C(O)/C=C/C(=O)N1CCc2ccc(Br)cc21]

To a stirred solution of ethyl (2E)-4-(6-bromo-2,3-dihydroindol-1-yl)-4-oxobut-2-enoate (500 mg, 1.5 mmol, 1.0 equiv) in ACN (5.0 mL) and H₂O (0.10 mL), lithium bromide (1300 mg, 15 mmol, 10 equiv) and Et₃N (1600 mg, 15 mmol, 10 equiv) was added at room temperature. The resulting mixture was stirred for 3.0 h at 50° C., and was concentrated under reduced pressure. The residue was dissolved in H₂O (10 mL) and acidified to pH 3-5 with 2 M HCl. The precipitated solids were collected by filtration and washed with H₂O (3×2.00 mL), and dried under vacuum to yield (2E)-4-(6-bromo-2,3-dihydroindol-1-yl)-4-oxobut-2-enoic acid (420 mg, 90%) as a yellow solid.

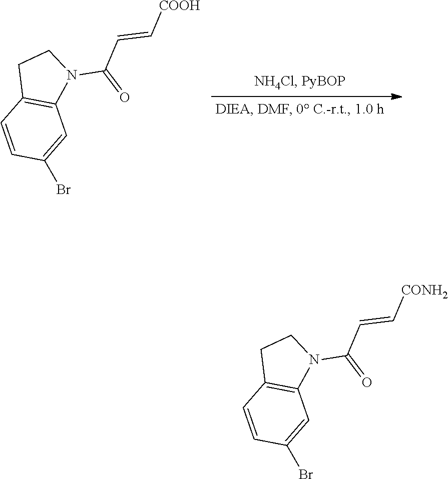 [SMILES: O=C(O)/C=C/C(=O)N1CCc2ccc(Br)cc21 ; NC(=O)/C=C/C(=O)N1CCc2ccc(Br)cc21]

To a stirred solution of (2E)-4-(6-bromo-2,3-dihydroindol-1-yl)-4-oxobut-2-enoic acid (400 mg, 1.4 mmol, 1.0 equiv) in DMF (5.0 mL), NH₄Cl (72 mg, 1.4 mmol, 1.0 equiv), PyBOP (1100 mg, 2.0 mmol, 1.5 equiv) and DIEA (0.71 mL, 4.1 mmol, 3.0 equiv) was added at 0° C. The resulting mixture was stirred for 1.0 h at room temperature. The reaction was poured into ice/water (15 mL) and the precipitated solids were collected by filtration and washed with H₂O (3×5.00 mL). The solids were then dried under vacuum to yield (2E)-4-(6-bromo-2,3-dihydroindol-1-yl)-4-oxobut-2-enamide (350 mg, 82%) as a yellow solid.

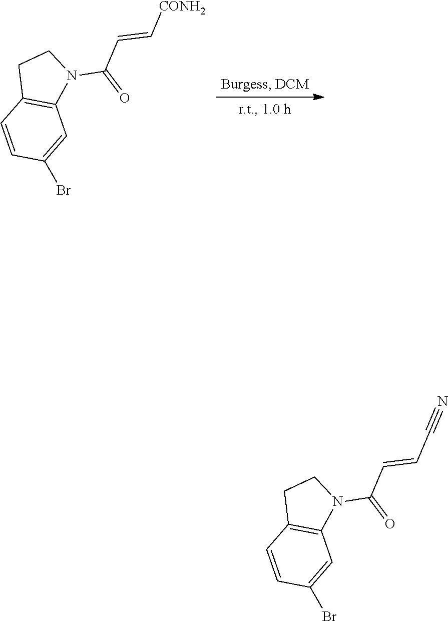 [SMILES: NC(=O)/C=C/C(=O)N1CCc2ccc(Br)cc21 ; N#C/C=C/C(=O)N1CCc2ccc(Br)cc21]

To a stirred solution of (2E)-4-(6-bromo-2,3-dihydroindol-1-yl)-4-oxobut-2-enamide (330 mg, 1.1 mmol, 1.0 equiv) in DCM (8.0 mL), Burgess reagent (800 mg, 3.4 mmol, 3.0 equiv) was added at room temperature. The resulting mixture was stirred for 1.0 h at room temperature and subsequently concentrated under vacuum. The residue was purified by silica gel column chromatography, and eluted with PE/EtOAc (1:1) to yield (2E)-4-(6-bromo-2,3-dihydroindol-1-yl)-4-oxobut-2-enenitrile (280 mg, 86%) as a yellow solid.

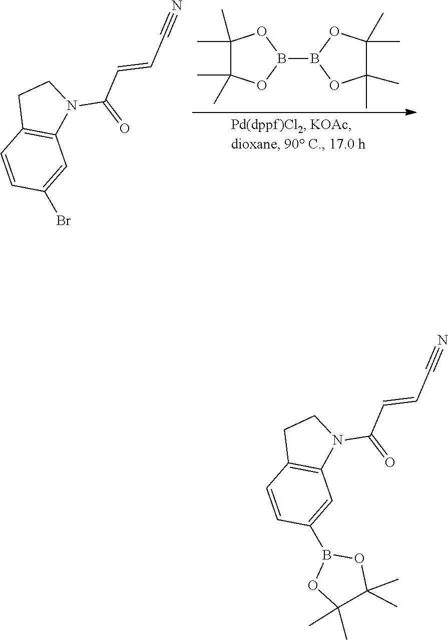

To a solution of (2E)-4-(6-bromo-2,3-dihydroindol-1-yl)-4-oxobut-2-enenitrile (260 mg, 0.94 mmol, 1.0 equiv) in 1,4-dioxane (3.0 mL), bis(pinacolato)diboron (360 mg, 1.4 mmol, 1.5 equiv), Pd(dppf)Cl₂·CH₂Cl₂ (150 mg, 0.19 mmol, 0.20 equiv) and KOAc (180 mg, 1.9 mmol, 2.0 equiv) was added at room temperature. The reaction was stirred at 90° C. for 17 h under N₂ atmosphere, in which the reaction was filtered, and the filtrate was concentrated. The residue was purified by silica gel column chromatography, eluted with PE/EtOAc (3:1) to yield (2E)-4-oxo-4-[6-(4,4,5,5-tetramethyl-1,3,2-dioxaborolan-2-yl)-2,3-dihydroindol-1-yl]but-2-enenitrile (100 mg, 33%) as a yellow green solid.

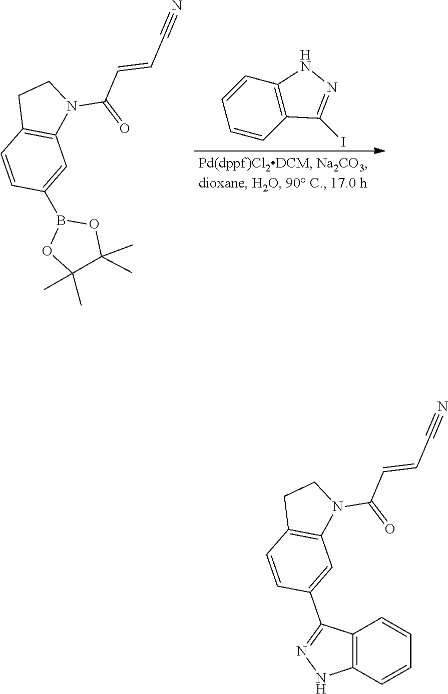 [SMILES: CC1(C)OB(c2ccc3c(c2)N(C(=O)/C=C/C#N)CC3)OC1(C)C ; Ic1n[nH]c2ccccc12 ; N#C/C=C/C(=O)N1CCc2ccc(-c3n[nH]c4ccccc34)cc21]

To a solution of (2E)-4-oxo-4-[6-(4,4,5,5-tetramethyl-1,3,2-dioxaborolan-2-yl)-2,3-dihydroindol-1-yl]but-2-enenitrile (60 mg, 0.19 mmol, 1.0 equiv) in 1,4-dioxane (2.0 mL) and H₂O (0.50 mL), 3-iodo-1H-indazole (45 mg, 0.19 mmol, 1.0 equiv), Pd(dppf)Cl₂·CH₂Cl₂ (30 mg, 0.04 mmol, 0.20 equiv) and Na₂CO₃ (98 mg, 0.93 mmol, 5.0 equiv) was added at room temperature. The reaction was stirred at 90° C. overnight under N₂ atmosphere and filtered. The filtration in DMF (3.0 mL) was purified by Prep-HPLC: Column: XBridge Prep Phenyl OBD Column, 19*250 mm, 5 μm; Mobile Phase A: Water (10 mmol/L NH₄HCO₃+0.1% NH₃·H₂O), Mobile Phase B: ACN; Flow rate: 25 mL/min; Gradient: 40% B to 70% B in 7 min, 70% B; wavelength: 254 nm; RT1(min): 5. The fractions were combined and lyophilized to yield (2E)-4-[6-(1H-indazol-3-yl)-2,3-dihydroindol-1-yl]-4-oxobut-2-enenitrile (6.9 mg, 12%) was obtained as yellow green solid.

LC/MS: mass calcd. For C₁₉H₁₄N₄O: 314.12, found: 315.05 [M+H]⁺.

¹H NMR (300 MHz, DMSO) δ: 13.23 (s, 1H), 8.86 (s, 1H), 8.02 (d, J=8.1 Hz, 1H), 7.66-7.75 (m, 2H), 7.60 (d, J=8.4 Hz, 1H), 7.38-7.44 (m, 2H), 7.22 (t, J=7.2 Hz, 1H), 6.75 (d, J=15.9 Hz, 1H), 4.37 (t, J=8.4 Hz, 2H), 3.25 (t, J=8.4 Hz, 2H).

### Example 13. Synthesis of 1-[2-methyl-6-{1H-pyrazolo [3,4-b]pyridin-3-yl}-2,3-dihydroindol-1-yl]prop-2-en-1-one (Compound 15)

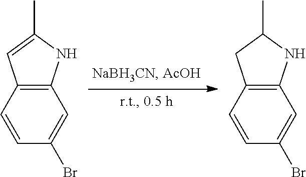

To a stirred solution of 6-bromo-2-methyl-1H-indole (1.5 g, 7.1 mmol, 1.0 equiv) in AcOH (30 mL), NaBH₃CN (2.2 g, 36 mmol, 5.0 equiv) was added at 0° C., with stirring for 0.5 h at room temperature. The reaction mixture was poured into water (30 mL) and extracted by EA (3×30 mL), the organic phases were combined and washed by H₂O (1×30 mL) and NaCl (1×30 mL) and dried over anhydrous Na₂SO₄. The filtrate was concentrated under reduced pressure and the residue was purified by silica gel column chromatography, eluted with PE/EtOAc (5:1) to afford 6-bromo-2-methyl-2,3-dihydro-1H-indole (1.3 g, 84%) as a white solid.

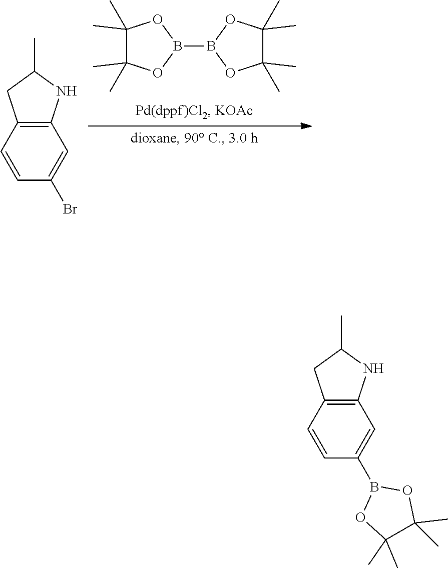

To a solution of 6-bromo-2-methyl-2,3-dihydro-1H-indole (1.3 g, 6.0 mmol, 1.0 equiv) in 1,4-dioxane (10 mL), bis(pinacolato)diboron (2.3 g, 9.0 mmol, 1.5 equiv), Pd(dppf)Cl₂·CH₂Cl₂ (0.98 g, 1.2 mmol, 0.20 equiv) and KOAc (1.2 g, 12 mmol, 2.0 equiv) was added at room temperature. Then the reaction was stirred at 90° C. for 3.0 h under N₂ atmosphere and subsequently filtered and the filtrate was concentrated. The residue was purified by silica gel column chromatography, and eluted with PE/EtOAc (5:1) to yield 2-methyl-6-(4,4,5,5-tetramethyl-1,3,2-dioxaborolan-2-yl)-2,3-dihydro-1H-indole (1.4 g, 90%) as a white solid.

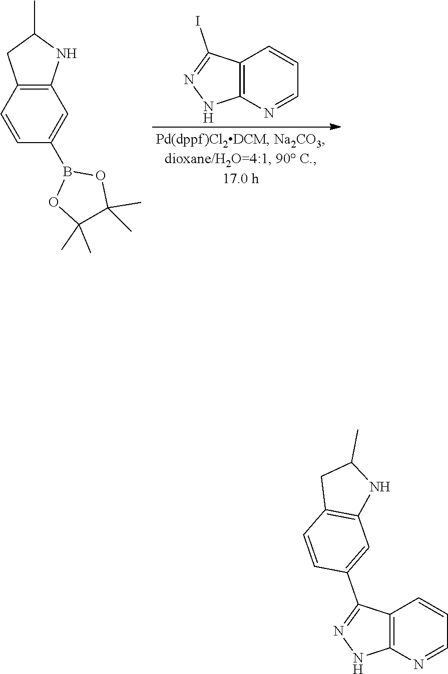 [SMILES: CC1Cc2ccc(B3OC(C)(C)C(C)(C)O3)cc2N1 ; Ic1n[nH]c2ncccc12 ; CC1Cc2ccc(-c3n[nH]c4ncccc34)cc2N1]

To a solution of 2-methyl-6-(4,4,5,5-tetramethyl-1,3,2-dioxaborolan-2-yl)-2,3-dihydro-1H-indole (1.4 g, 5.3 mmol, 1.0 equiv) in 1,4-dioxane (8.0 mL) and H₂O (2.0 mL), 3-iodo-1H-pyrazolo[3,4-b]pyridine (1.4 g, 5.8 mmol, 1.1 equiv), Pd(dppf)Cl₂·CH₂Cl₂ (0.86 g, 1.1 mmol, 0.20 equiv) and Na₂CO₃ (2.8 g, 26 mmol, 5.0 equiv) was added at room temperature. Then the reaction was stirred at 90° C. for 17 h under N₂ atmosphere. The reaction mixture was filtered, and the filtrate was concentrated. The residue was purified by silica gel column chromatography, and eluted with PE/EtOAc (1:1) to yield 2-methyl-6-{1H-pyrazolo[3,4-b]pyridin-3-yl}-2,3-dihydro-1H-indole (370 mg, 52%) as a white solid.

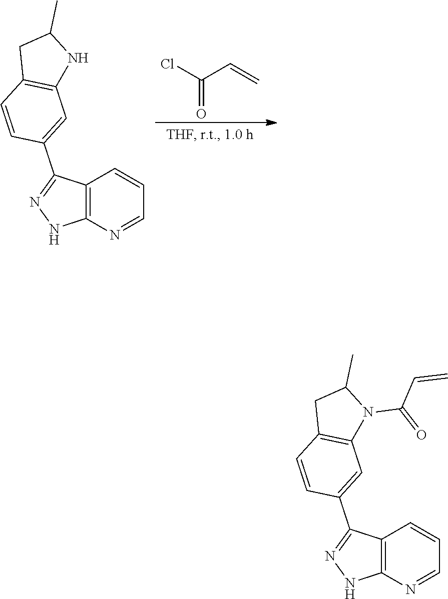

To a stirred solution of 2-methyl-6-{1H-pyrazolo[3,4-b]pyridin-3-yl}-2,3-dihydro-1H-indole (200 mg, 0.80 mmol, 1.0 equiv) in THE (5.0 mL), acryloyl chloride (87 mg, 0.96 mmol, 1.2 equiv) was added at 0° C. The resulting mixture was stirred for 1.0 h at room temperature. The resulting mixture was concentrated under vacuum and purified by reverse phase column directly with the following conditions: column: C18 silica gel; mobile phase, ACN in water (0.05% TFA), 40% to 50% gradient in 30 min; detector, UV 254 nm. The fractions were combined and concentrated to yield 170 mg of 1-[2-methyl-6-{1H-pyrazolo [3,4-b]pyridin-3-yl}-2,3-dihydroindol-1-yl]prop-2-en-1-one (70%) as an orange solid.

LC/MS: mass calcd. For C₁₈H₁₆N₄O: 304.13, found: 305.15 [M+H]⁺.

¹H NMR (400 MHz, DMSO) δ: 13.74 (brs, 1H), 8.85 (s, 1H), 8.58 (s, 1H), 8.48 (d, J=8.0 Hz, 1H), 7.72 (d, J=8.0 Hz, 1H), 7.43 (d, J=8.0 Hz, 1H), 7.29-7.33 (m, 1H), 6.83-6.90 (m, 1H), 6.39 (d, J=16.8 Hz, 1H), 5.88 (d, J=10.5 Hz, 1H), 4.88-4.91 (m, 1H), 3.44-3.50 (m, 1H), 2.81-2.89 (m, 1H), 1.26 (s, 3H).

### Example 14. Synthesis of 1-[(2S)-2-methyl-6-{1H-pyrazolo [3,4-b]pyridin-3-yl}-2,3-dihydroindol-1-yl]prop-2-en-1-one (Compounds 15-A and 15-B)

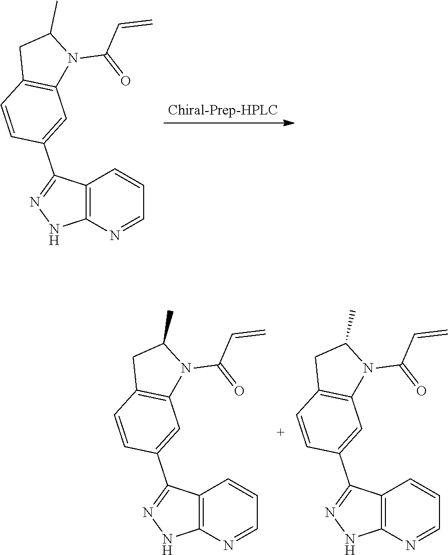 [SMILES: C=CC(=O)N1c2cc(-c3n[nH]c4ncccc34)ccc2CC1C ; C=CC(=O)N1c2cc(-c3n[nH]c4ncccc34)ccc2C[C@H]1C ; C=CC(=O)N1c2cc(-c3n[nH]c4ncccc34)ccc2C[C@@H]1C]

150 mg of 1-(2-methyl-6-{1H-pyrazolo[3,4-b]pyridin-3-yl}-2,3-dihydroindol-1-yl)prop-2-en-1-one in MeOH (3.0 mL) was purified by silica gel column chromatography: column: CHIRAL ART Amylose-SA, 2*25 cm, 5 μm; Mobile Phase A: Hex(0.5% 2M NH₃-MeOH)—HPLC, Mobile Phase B: EtOH—HPLC; Flow rate: 20 mL/min; Gradient: 30% B to 30% B in 15 min; wavelength: 220/254 nm; RT1 for isomer A (min): 6.6; RT2 for isomer B (min): 8.3; sample solvent: EtOH—HPLC; injection volume: 1 mL. Absolute stereochemistry of isomer A and isomer B were not determined. After lyophilization, both isomers were obtained as white solids (yield: 31 mg, 13% and 31 mg, 13%).

### Data for Compound 15-A:

LC/MS: mass calcd. For C₁₈H₁₆N₄O: 304.13, found: 305.15 [M+H]⁺.

¹H NMR (400 MHz, DMSO) δ: 13.79 (s, 1H), 8.85 (s, 1H), 8.58 (d, J=6.0 Hz, 1H), 8.48 (d, J=8.0 Hz, 1H), 7.73 (d, J=7.6 Hz, 1H), 7.43 (d, J=8.0 Hz, 1H), 7.29-7.32 (m, 1H), 6.83-6.90 (m, 1H), 6.39 (d, J=16.4 Hz, 1H), 5.88 (d, J=10.5 Hz, 1H), 4.88-4.92 (m, 1H), 3.44-3.50 (m, 1H), 2.77-2.81 (m, 1H), 1.27 (s, 3H).

### Data for Compound 15-B

LC/MS: mass calcd. For C₁₈H₁₆N₄O: 304.13, found: 305.15 [M+H]⁺.

¹H NMR (400 MHz, DMSO) δ: 13.79 (s, 1H), 8.85 (s, 1H), 8.58 (d, J=6.0 Hz, 1H), 8.48 (d, J=8.0 Hz, 1H), 7.73 (d, J=7.6 Hz, 1H), 7.43 (d, J=8.0 Hz, 1H), 7.29-7.32 (m, 1H), 6.83-6.90 (m, 1H), 6.39 (d, J=16.4 Hz, 1H), 5.88 (d, J=10.5 Hz, 1H), 4.88-4.92 (m, 1H), 3.44-3.50 (m, 1H), 2.77-2.81 (m, 1H), 1.27 (s, 3H).

### Example 15. Synthesis of 1-(7-fluoro-6-{1H-pyrazolo[3,4-b]pyridin-3-yl}-2,3-dihydroindol-1-yl)prop-2-en-1-one (Compound 17)

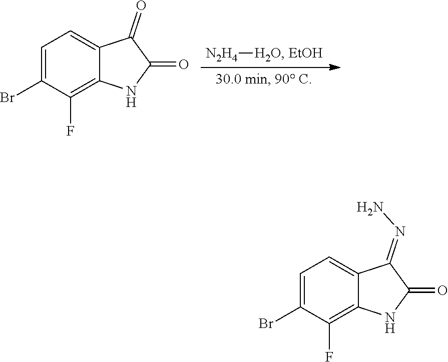

To a stirred solution of 6-bromo-7-fluoro-1H-indole-2,3-dione (3.0 g, 12 mmol, 1.0 equiv) in EtOH (60 mL), NH₂NH₂·H₂O (0.60 mL, 12 mmol, 1.0 equiv) was added at room temperature. The resulting mixture was stirred for 30 min at 90° C. and the precipitated solids were collected by filtration and washed with EtOH (3×10 mL). The solids were purified by silica gel column chromatography, eluted with DCM/MeOH(15:1), to yield (3E)-6-bromo-7-fluoro-3-hydrazinylidene-1H-indol-2-one (2.2 g, 69%) as a yellow solid.

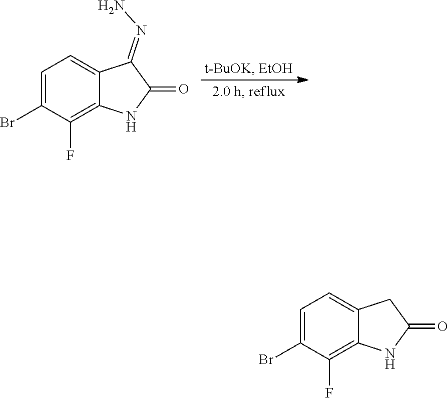

To a solution of (3E)-6-bromo-7-fluoro-3-hydrazinylidene-1H-indol-2-one (1.8 g, 7.0 mmol, 1.0 equiv) in EtOH (40 mL), t-BuOK (2.4 g, 21 mmol, 3.0 equiv) was added at room temperature. The mixture was refluxed under nitrogen atmosphere for 2.0 h before pouring into water/ice. The mixture was acidified to pH=2 with diluted HCl (2M). The precipitated solids were collected by filtration and washed with H₂O (3×10.00 mL), dried under vacuum to afford 6-bromo-7-fluoro-1,3-dihydroindol-2-one (1.4 g, 76%) as a yellow solid.

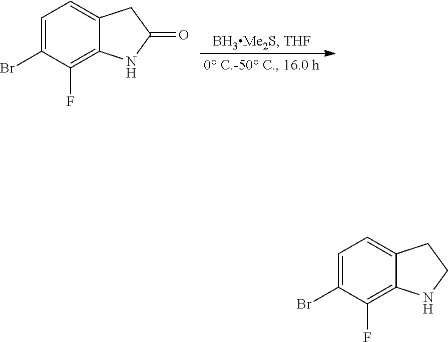

To a solution of 6-bromo-7-fluoro-1,3-dihydroindol-2-one (900 mg, 3.9 mmol, 1.0 equiv) in THE (8.0 mL), NaBH₄ (440 mg, 12 mmol, 3.0 equiv) and BF₃·Et₂O (1700 mg, 12 mmol, 3.0 equiv) was added at 0° C. The reaction was stirred at 50° C. for 16 h, and the pH value of the mixture was adjusted to pH 7-8 with NaHCO₃ (aq.) at 0° C. The mixture was extracted by EA (3×5 mL), the organic phases were combined and washed with H₂O (1×5 mL) and NaCl (1×5 mL) and dried over anhydrous Na₂SO₄. The filtrate was concentrated under reduced pressure, and the residue was purified by silica gel column chromatography, eluted with PE/EtOAc (1:1) to afford 6-bromo-7-fluoro-2,3-dihydro-1H-indole (810 mg, 81%), as a yellow solid.

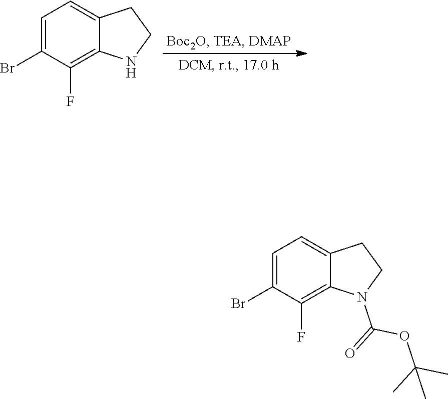

To a stirred solution of 6-bromo-7-fluoro-2,3-dihydro-1H-indole (700 mg, 3.20 mmol, 1.0 equiv) in DCM (10 mL), Boc₂O (710 mg, 3.2 mmol, 1.0 equiv), Et₃N (980 mg, 9.7 mmol, 3.0 equiv) and DMAP (40 mg, 0.32 mmol, 0.10 equiv) was added at 0° C. The resulting mixture was stirred for 17 h at room temperature. The reaction mixture was poured into ice/water (10 mL). The reaction mixture was extracted with DCM (3×20 mL). The combined organic phase was washed with H₂O (1×10 mL) and NaCl (1×10 mL), and dried over anhydrous Na₂SO₄. The filtrate was concentrated under reduced pressure, and the residue was purified by silica gel column chromatography, eluted with PE/EtOAc (10:1), to yield tert-butyl 6-bromo-7-fluoro-2,3-dihydroindole-1-carboxylate (530 mg, 48%) as a yellow solid.

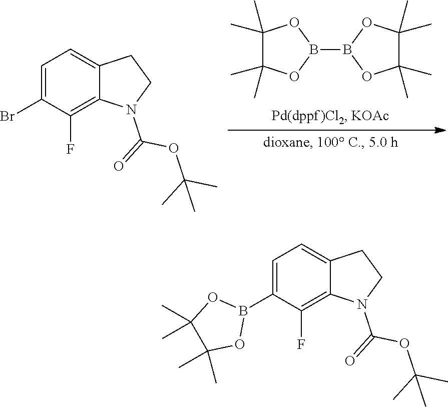

To a stirred solution of tert-butyl 6-bromo-7-fluoro-2,3-dihydroindole-1-carboxylate (500 mg, 1.6 mmol, 1.0 equiv) in 1,4-dioxane (8.0 mL), bis(pinacolato)diboron (600 mg, 2.4 mmol, 1.5 equiv) and Pd(dppf)Cl₂CH₂Cl₂ (260 mg, 0.32 mmol, 0.20 equiv) was added under nitrogen atmosphere. KOAc (310 mg, 3.2 mmol, 2.0 equiv) was added and the resulting mixture was stirred for 5.0 h at 100° C. under nitrogen atmosphere. The reaction mixture was filtered, and the filtration was concentrated and purified by silica gel column chromatography, eluted with PE/EtOAc (1:5), to yield tert-butyl 7-fluoro-6-(4,4,5,5-tetramethyl-1,3,2-dioxaborolan-2-yl)-2,3-dihydroindole-1-carboxylate (470 mg, 63%) as a yellow solid.

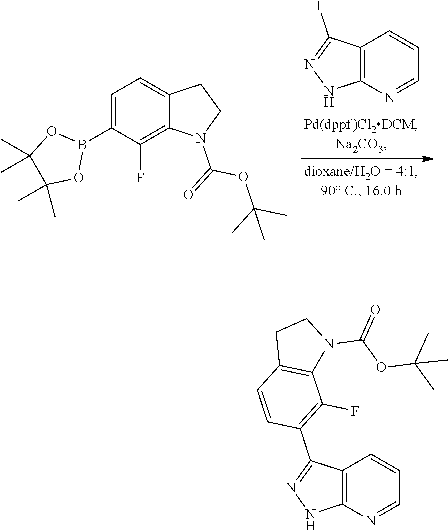 [SMILES: CC(C)(C)OC(=O)N1CCc2ccc(B3OC(C)(C)C(C)(C)O3)c(F)c21 ; Ic1n[nH]c2ncccc12 ; CC(C)(C)OC(=O)N1CCc2ccc(-c3n[nH]c4ncccc34)c(F)c21]

To a solution of tert-butyl-7-fluoro-6-(4,4,5,5-tetramethyl-1,3,2-dioxaborolan-2-yl)-2,3-dihydroindole-1-carboxylate (460 mg, 1.3 mmol, 1.0 equiv) in 1,4-dioxane (4.0 mL) and H₂O (1.0 mL), 3-iodo-1H-pyrazolo[3,4-b]pyridine (370 mg, 1.5 mmol, 1.2 equiv), Pd(dppf)Cl₂·CH₂Cl₂ (210 mg, 0.25 mmol, 0.20 equiv) and Na₂CO₃ (670 mg, 6.3 mmol, 5.0 equiv) was added at room temperature. Then the reaction was stirred at 90° C. for 16 h under nitrogen atmosphere. The reaction mixture was filtered and the filtrate was concentrated. The residue was purified by silica gel column chromatography, eluted with PE/EtOAc (1:1), to yield tert-butyl 7-fluoro-6-{1H-pyrazolo[3,4-b]pyridin-3-yl}-2,3-dihydroindole-1-carboxylate (380 mg, 84%) as a white solid.

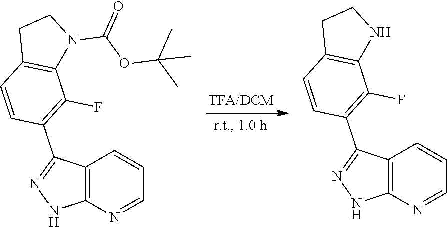 [SMILES: CC(C)(C)OC(=O)N1CCc2ccc(-c3n[nH]c4ncccc34)c(F)c21 ; Fc1c(-c2n[nH]c3ncccc23)ccc2c1NCC2]

To a stirred solution of tert-butyl 7-fluoro-6-{1H-pyrazolo[3,4-b]pyridin-3-yl}-2,3-dihydroindole- 1-carboxylate (190 mg, 0.54 mmol, 1.0 equiv) in DCM (5.0 mL), TFA (1.0 mL) was added dropwise at room temperature. The resulting mixture was stirred for 1.0 h at room temperature. The resulting mixture was concentrated under vacuum. 7-fluoro-6-{1H-pyrazolo[3,4-b]pyridin-3-yl}-2,3-dihydro-1H-indole (190 mg, crude) was obtained as a yellow oil.

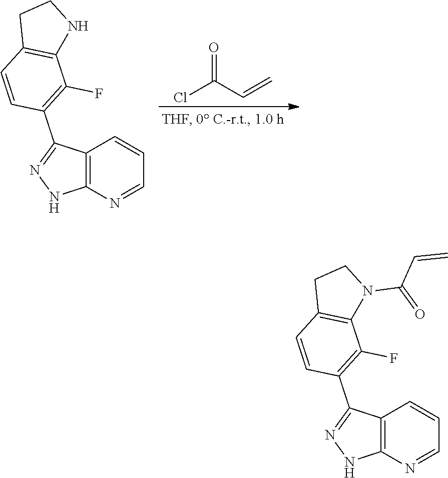

To a stirred solution of 7-fluoro-6-{1H-pyrazolo[3,4-b]pyridin-3-yl}-2,3-dihydro-1H-indole (130 mg, 0.51 mmol, 1.0 equiv) in THE (5.0 mL), acryloyl chloride (42 mg, 0.46 mmol, 0.90 equiv) was added at 0° C. The resulting mixture was stirred for 1.0 h at room temperature. The reaction mixture was concentrated and the residue was dissolved in DMF (3.0 mL) and filtered. The filtrate was purified by Prep-HPLC: column: XBridge Prep Phenyl OBD Column, 19*250 mm, 5 μm; Mobile Phase A: Water (10 mmol/L NH₄HCO₃+0.1% NH₃·H₂O), Mobile Phase B: MeOH—HPLC; Flow rate: 25 mL/min; Gradient: 44% B to 74% B in 15 min, 74% B; wavelength: 254 nm; RT1(min): 12. The fractions were combined and lyophilized, and 1-(7-fluoro-6-{1H-pyrazolo[3,4-b]pyridin-3-yl}-2,3-dihydroindol-1-yl)prop-2-en-1-one (20 mg, 12%) was yielded as white solid.

LC/MS: mass calcd. For C₁₇H₁₃FN₄O: 308.11, found: 309.10 [M+H]⁺.

¹H NMR (300 MHz, DMSO) δ: 13.96 (s, 1H), 8.58 (d, J=6.0 Hz, 1H), 8.20-8.24 (m, 1H), 7.54 (t, J=6.3 Hz, 1H), 7.26-7.31 (m, 2H), 6.66-6.76 (m, 1H), 6.30 (d, J=16.8 Hz, 1H), 5.86 (d, J=10.5 Hz, 1H), 4.25 (t, J=7.8 Hz, 2H), 3.20 (t, J=7.8 Hz, 2H).

### Example 16. Synthesis of 1-(ethenesulfonyl)-6-{1H-pyrazolo[3,4-b]pyridin-3-yl}-2,3-dihydroindole (Compound 16)

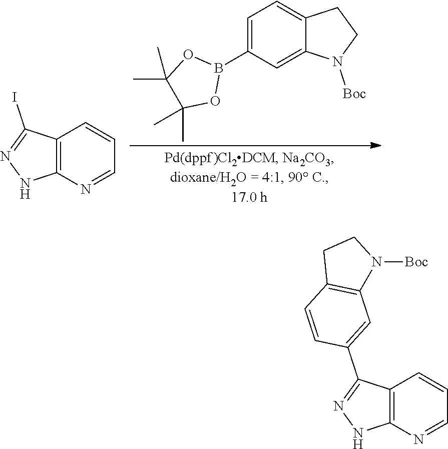

To a solution of 3-iodo-1H-pyrazolo[3,4-b]pyridine (3.0 g, 12 mmol, 1.0 equiv) in 1,4-dioxane (80 mL) and H₂O (20 mL), tert-butyl 6-(4,4,5,5-tetramethyl-1,3,2-dioxaborolan-2-yl)-2,3-dihydroindole-1-carboxylate (5.9 g, 17 mmol, 1.4 equiv), Pd(dppf)Cl₂·CH₂Cl₂ (2.0 g, 2.5 mmol, 0.20 equiv) and Na₂CO₃ (6.5 g, 61 mmol, 5.0 equiv) was added with stirring at 90° C. for 17 h under nitrogen atmosphere. The reaction mixture was filtered, and the filtrate was concentrated. The residue was purified by silica gel column chromatography, eluted with PE/EA (10:1), to yield tert-butyl 6-{1H-pyrazolo[3,4-b]pyridin-3-yl}-2,3-dihydroindole-1-carboxylate (1.6 g, 35%) as a yellow oil.

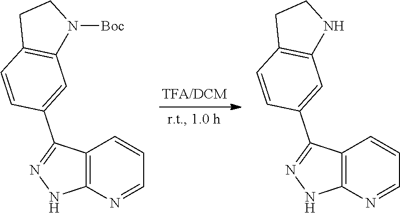 [SMILES: CC(C)(C)OC(=O)N1CCc2ccc(-c3n[nH]c4ncccc34)cc21 ; c1cnc2[nH]nc(-c3ccc4c(c3)NCC4)c2c1]

To a stirred solution of tert-butyl 6-{1H-pyrazolo[3,4-b]pyridin-3-yl}-2,3-dihydroindole-1-carboxylate (660 mg, 2.0 mmol, 1.0 equiv) in DCM (8.0 mL), TFA (2.0 mL) was added dropwise at room temperature. The resulting mixture was stirred for 1.0 h at room temperature. The resulting mixture was concentrated under vacuum to afford 6-{1H-pyrazolo[3,4-b]pyridin-3-yl}-2,3-dihydro-1H-indole (660 mg, crude) as a yellow oil.

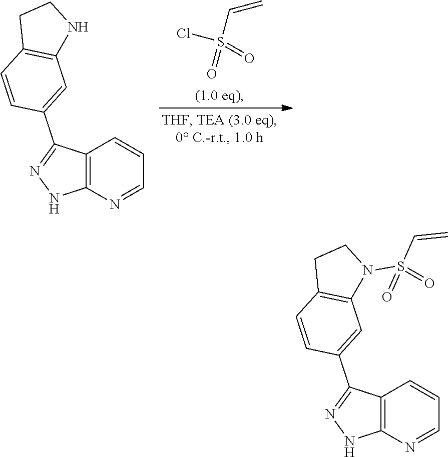

To 6-{1H-pyrazolo[3,4-b]pyridin-3-yl}-2,3-dihydro-1H-indole (200 mg, 0.85 mmol, 1.0 equiv) in THE (10 mL), TEA (260 mg, 2.6 mmol, 3.0 equiv) was added. This was followed by the addition of ethenesulfonyl chloride (110 mg, 0.85 mmol, 1.0 equiv) at 0° C. The resulting mixture was stirred at 25° C. for 1.0 hours. The reaction was quenched with ice/water (20 mL) and extracted with DCM (3×20 mL).

The combined extracts were dried over anhydrous Na₂SO₄, and the solid was filtered out and concentrated. The crude was purified by Prep-HPLC with the following conditions: Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 27% B to 57% B in 7 min, 57% B; wavelength: 254 nm; RT1(min): 5.4. After lyophilization, 1-(ethenesulfonyl)-6-{1H-pyrazolo[3,4-b]pyridin-3-yl}-2,3-dihydroindole was yielded as a yellow solid (20 mg, 7.3% yield).

LC/MS: mass calcd. For C₁₆H₁₄N₄O₂S: 326.08, found: 327.15 [M+H]⁺.

¹H NMR (300 MHz, DMSO) δ:13.83 (brs, 1H), 8.58 (d, J=4.5 Hz, 1H), 8.45 (d, J=8.4 Hz, 1H), 7.89 (s, 1H), 7.69 (d, J=7.8 Hz, 1H), 7.41 (d, J=7.8 Hz, 1H), 7.29-7.33 (m, 1H), 6.92-7.01 (m, 1H), 6.19-6.32 (m, 2H), 3.99 (t, J=8.4 Hz, 2H), 3.16 (t, J=8.4 Hz, 2H).

### Example 17. Synthesis of 1-[6-(1H-indazol-3-yl)-2,3-dihydroindol-1-yl]prop-2-en-1-one (Compound 14)

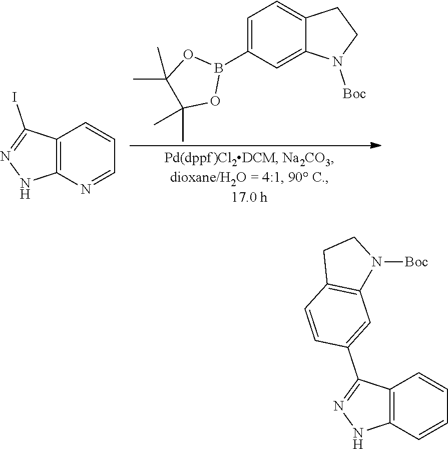

To a solution of 3-iodo-1H-indazole (500 mg, 2.1 mmol, 1.0 equiv) in 1,4-dioxane (10 mL) and H₂O (2.5 mL), tert-butyl 6-(4,4,5,5-tetramethyl-1,3,2-dioxaborolan-2-yl)-2,3-dihydroindole-1-carboxylate (850 mg, 2.5 mmol, 1.2 equiv), Pd(dppf)Cl₂·CH₂Cl₂ (170 mg, 0.21 mmol, 0.10 equiv) and Na₂CO₃ (1100 mg, 10 mmol, 5.0 equiv) was added. The reaction was stirred at 90° C. for 17 h. The reaction mixture was filtered, and the filtrate was concentrated. The residue was purified by silica gel column chromatography, eluted with DCM/MeOH(40:1), to yield tert-butyl 6-(1H-indazol-3-yl)-2,3-dihydroindole-1-carboxylate (710 mg, 98%) as a yellow oil.

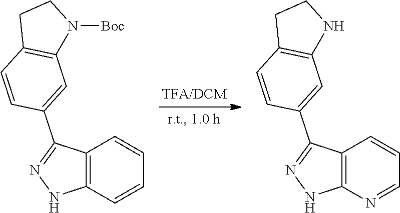 [SMILES: CC(C)(C)OC(=O)N1CCc2ccc(-c3n[nH]c4ccccc34)cc21 ; c1cnc2[nH]nc(-c3ccc4c(c3)NCC4)c2c1]

To a stirred solution of tert-butyl 6-(1H-indazol-3-yl)-2,3-dihydroindole-1-carboxylate (550 mg, 1.6 mmol, 1.0 equiv) in DCM (5.0 mL), TFA (1.3 mL) was added dropwise at room temperature. The resulting mixture was stirred for 1.0 h at room temperature. The reaction mixture was concentrated. 500 mg crude was obtained. 100 mg was purified by Prep-HPLC directly with the following conditions: Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 28% B to 58% B in 7 min, 58% B; wavelength: 254 nm; RT1(min): 5.2. The fractions were combined and lyophilized to yield 3-(2,3-dihydro-1H-indol-6-yl)-1H-indazole (5.1 mg) as a white solid.

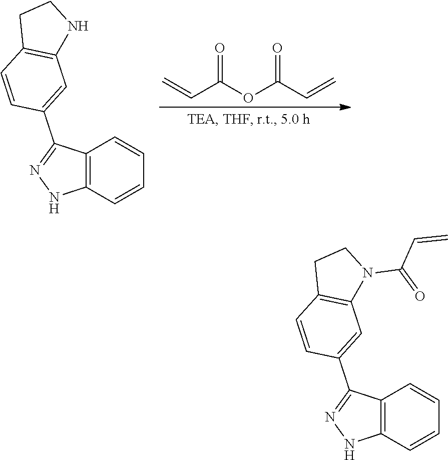

To a stirred solution of 3-(2,3-dihydro-1H-indol-6-yl)-1H-indazole (70 mg, 0.30 mmol, 1.0 equiv) in THE (3.0 mL), prop-2-enoyl prop-2-enoate (110 mg, 0.89 mmol, 3.0 equiv) and Et₃N (240 mg, 2.4 mmol, 8.0 equiv) was added in portions at 0° C. The resulting mixture was stirred for 5.0 h at room temperature. The reaction mixture was concentrated and residue was dissolved in DMF (1.0 mL) and purified by Prep-HPLC with the following conditions: Column: XBridge Prep OBD C18 Column, 30*150 mm, 5 μm; Mobile Phase A: Water (0.1% FA), Mobile Phase B: ACN; Flow rate: 60 mL/min; Gradient: 25% B to 55% B in 7 min, 55% B; Wavelength: 254 nm; RT1(min): 6.75. The fractions were combined and lyophilized. 1-[6-(1H-indazol-3-yl)-2,3-dihydroindol-1-yl]prop-2-en-1-one (5.9 mg, 6.7%) was obtained as a white solid.

LC/MS: mass calcd. For C₁₈H₁₅N₃O: 289.12, found: 290.10 [M+H]⁺.

¹H NMR (300 MHz, DMSO) 5:13.22 (s, 1H), 8.88 (s, 1H), 8.03 (d, J=8.1 Hz, 1H), 7.58-7.69 (m, 2H), 7.37-7.43 (m, 2H), 7.23 (t, J=7.5 Hz, 1H), 6.75-6.84 (m, 1H), 6.35 (d, J=16.5 Hz, 1H), 5.85 (d, J=10.5 Hz, 1H), 4.29 (t, J=8.1 Hz, 2H), 3.23 (t, J=8.1 Hz, 2H).

### Example 18. Synthesis of 3-[1-(prop-2-enoyl)-2,3-dihydroindol-6-yl]-1H-indazole-6-carbonitrile (Compound 18)

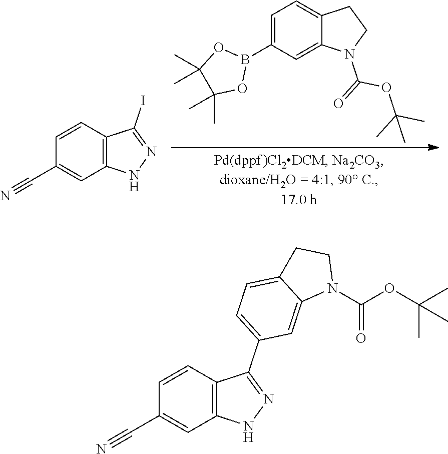

To a solution of 3-iodo-1H-indazole-6-carbonitrile (500 mg, 1.9 mmol, 1.0 equiv) in 1,4-dioxane (8.0 mL) and H₂O (2.0 mL), tert-butyl 6-(4,4,5,5-tetramethyl-1,3,2-dioxaborolan-2-yl)-2,3-dihydroindole-1-carboxylate (770 mg, 2.2 mmol, 1.2 equiv), Pd(dppf)Cl₂·CH₂Cl₂ (300 mg, 0.37 mmol, 0.20 equiv) and Na₂CO₃ (980 mg, 9.3 mmol, 5.0 equiv) was added. The resulting mixture was stirred for 17 h at 90° C. After filtration, the filtrate was concentrated under reduced pressure. The residue was purified by TLC-Plate with PE/EtOAc (10:1) to afford tert-butyl 6-(6-cyano-1H-indazol-3-yl)-2,3-dihydroindole-1-carboxylate (450 mg, 57%) as a yellow solid.

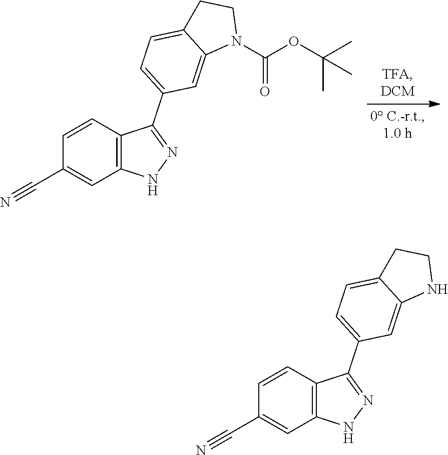 [SMILES: CC(C)(C)OC(=O)N1CCc2ccc(-c3n[nH]c4cc(C#N)ccc34)cc21 ; N#Cc1ccc2c(-c3ccc4c(c3)NCC4)n[nH]c2c1]

To a stirred solution of tert-butyl 6-(6-cyano-1H-indazol-3-yl)-2,3-dihydroindole-1-carboxylate (450 mg, 1.3 mmol, 1.0 equiv) in DCM (4.0 mL), TFA (1.0 mL) was added dropwise at room temperature. The resulting mixture was stirred for 1.0 h at room temperature. The resulting mixture was concentrated under vacuum. 3-(2,3-dihydro-1H-indol-6-yl)-1H-indazole-6-carbonitrile (450 mg, crude) was obtained as a yellow oil.

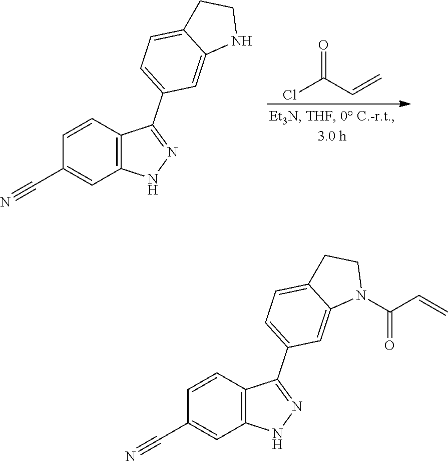

To a stirred solution of 3-(2,3-dihydro-1H-indol-6-yl)-1H-indazole-6-carbonitrile (200 mg, 0.77 mmol, 1.0 equiv) in THF (5.0 mL), Et₃N (160 mg, 1.5 mmol, 2.0 equiv) and acryloyl chloride (69 mg, 0.77 mmol, 1.0 equiv) was added dropwise at 0° C. The resulting mixture was stirred for 3.0 h at room temperature. The reaction mixture was concentrated and residue was dissolved in DMF (1.0 mL) and purified by Prep-HPLC with the following conditions: Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 33% B to 63% B in 7 min, 63% B; wavelength: 254 nm; RT1(min): 6. The fraction were combined and lyophilized. 3-[1-(prop-2-enoyl)-2,3-dihydroindol-6-yl]-1H-indazole-6-carbonitrile (19 mg, 7.8%) was obtained as a white solid.

LC/MS: mass calcd. For C₁₉H₁₄N₄O: 314.12, found: 315.10 [M+H]⁺.

¹H NMR (300 MHz, DMSO) δ:13.70 (s, 1H), 8.85 (s, 1H), 8.17-8.23 (m, 2H), 7.56-7.69 (m, 2H), 7.41 (d, J=7.8 Hz, 1H), 6.75-6.84 (m, 1H), 6.35 (d, J=16.8 Hz, 1H), 5.84 (t, J=10.5 Hz, 1H), 4.90 (t, J=8.1 Hz, 2H), 3.24 (t, J=8.1 Hz, 2H).

### Example 19. Synthesis of (2E)-4-[6-(1H-indazol-3-yl)-2,3-dihydroindol-1-yl]-4-oxobut-2-enoate (Compound 7)

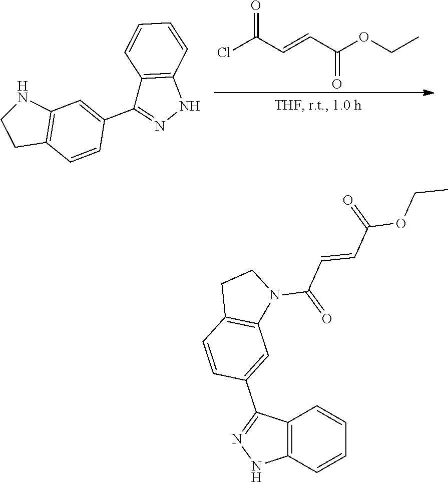

To a stirred solution of 3-(2,3-dihydro-1H-indol-6-yl)-1H-indazole (70 mg, 0.30 mmol, 1.0 equiv) in THF (1.5 mL), ethyl (2E)-4-chloro-4-oxobut-2-enoate (58 mg, 0.36 mmol, 1.2 equiv) was added at 0° C. The resulting mixture was stirred for 1.0 h at room temperature and filtered and the filtration in DMF (3.0 mL) was purified by Prep-HPLC: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 40% B to 70% B in 7 min, 70% B; wavelength: 254 nm; RT1(min): 6. The fractions were combined and lyophilized, yielding ethyl (2E)-4-[6-(1H-indazol-3-yl)-2,3-dihydroindol-1-yl]-4-oxobut-2-enoate (12 mg, 10%) as a yellow green solid.

LC/MS: mass calcd. For C₂₁H₁₉N₃O₃: 361.14, found: 362.15 [M+H]⁺.

¹H NMR (300 MHz, DMSO) δ: 13.22 (s, 1H), 8.88 (s, 1H), 8.02 (d, J=8.1 Hz, 1H), 7.72 (d, J=8.4 Hz, 1H), 7.60 (d, J=8.4 Hz, 1H), 7.39-7.46 (m, 3H), 7.23 (t, J=7.5 Hz, 1H), 6.78 (d, J=15.3 Hz, 1H), 4.38 (t, J=8.4 Hz, 2H), 4.21-4.28 (m, 2H), 3.22-3.32 (m, 2H), 1.28 (t, J=7.2 Hz, 3H).

### Example 20. In Vitro pharmacology of Compounds 1-18

A study was conducted to determine the pharmacology of Compounds 1-18 in vitro in a plurality of biological matrices. Compounds 1-18 were prepared into proper concentrations of DMSO solution. The compound stock was then serially diluted into 10 concentrations by 3-fold dilution in 384 pp-plate. 5 nL of compound stock was transferred to a 384 plate. The compound stock was then diluted to 0.74 nM using a solution, which contained 0.79 mL of water, 0.2 mL of 5× Buffer, 2 μL of DTT, 6.66 μL of 7.5% BSA, and 5 μL of 10 mM ATP, to form an MKK7 mixture. The MKK7 mixture was then pre-warmed and 2.5 μL was added to each well designated for the “Test compound” and “High control” well, 2.5 μL solution 1 to each well designated for the “Low control” well. The well was incubated for 30 min and centrifuged at room temperature. Following centrifugation, 5 μL of ADD-Glo Reagent was added to each well and incubated for 60 min after centrifugation at room temperature. 10 μL of ADP-Glo Detection was then added to each well and incubated for 30 min after centrifugation in dark at room temperature. IC₅₀ values were calculated based on the remaining activity (%) using read conversion ratio (CR).

$$\% Remaining Activity = 100 \% - 100 \% \times \frac{{S}_{H C} - {S}_{sample}}{{S}_{H C} - {S}_{L C}}$$

The results of the study are provided in Table 2.

TABLE 2

| Compound | IC₅₀ (nM) |
|---|---|
| 1 | 4.8 |
| 2 | 92 |
| 3 | 30 |
| 4 | 23 |
| 5 | 70 |
| 6 | 180 |
| 7 | 3900 |
| 8 | 840 |
| 9 | 1100 |
| 10 | 550 |
| 11 | 1400 |
| 12 | 160 |
| 13 | 140 |
| 14 | 19 |
| 15 | 440 |
| 15-B | 160 |
| 16 | 7.7 |
| 17 | 88 |
| 18 | 6.0 |

### Example 21. In Vitro Competitive Binding Assay of Compounds 1-18

Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 111× stocks in 100% DMSO. Kds were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each well has a final volume of 0.02 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The kinase concentration in the eluates was measured by qPCR.

An 11-point 3-fold serial dilution of each test compound was prepared in 100% DMSO at 100× final test concentration and subsequently diluted to 1× in the assay (final DMSO concentration=1%). Most Kds were determined using a compound top concentration=30,000 nM. If the initial Kd determined was <0.5 nM (the lowest concentration tested), the measurement was repeated with a serial dilution starting at a lower top concentration. A Kd value reported as 40,000 nM indicates that the Kd was determined to be >30,000 nM.

Binding constants (Kds) were calculated with a standard dose-response curve using the Hill equation:

$$Response = Background + \frac{Sign a l - B a c k g r o u n d}{1 + ( \frac{K {d}^{Hill Slope}}{{Dose}^{Hill Slope}} )}$$

The hill slope was set to −1. Curves were fitted using a non-linear least square fit with the levenberg-marquardt algorithm. The results of the study are provided in Table 3.

TABLE 3

| Compound | Kd (nM) |
|---|---|
| 4 | 7.8 |
| 12 | 71 |

### OTHER EMBODIMENTS

While the invention has been described in connection with specific embodiments thereof, it will be understood that it is capable of further modifications and this application is intended to cover any variations, uses, or adaptations of the invention following, in general, the principles of the disclosure and including such departures from the invention that come within known or customary practice within the art to which the disclosure pertains and may be applied to the essential features hereinbefore set forth, and follows in the scope of the claims. Other embodiments are within the claims.

1. A compound of formula (I): 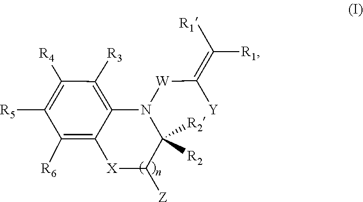
or a pharmaceutically acceptable salt thereof, wherein:
W is C(O) or S(O)₂, wherein one of R₁ or R₁′ is H, and the other R₁ or R₁′ is H; cyano; optionally substituted C₁-C₄ alkyl; CH₂N(Rₐ)₂, where each Rₐ is independently H or optionally substituted C₁-C₄ alkyl; or C(O)ORₐ′, wherein Rₐ′ is optionally substituted C₁-C₄ alkyl or H;
Y is halo or H;
each of R₂ and R₂′ is independently H, optionally substituted C₁-C₄ alkyl or optionally substituted C₃-C₆ cycloalkyl;
Z, if present, is C₁-C₄ alkyl;
n is 0 or 1;
X is C(R_{b})₂, O, or NR_{b}′;
each of R_{b} and R_{b}′ is independently H or optionally substituted C₁-C₄ alkyl;
each of R₃ and R₅ is independently H, halo, optionally substituted C₁-C₄ alkyl, optionally substituted C₁-C₄ alkoxy, optionally substituted C₁-C₄ haloalkyl, optionally substituted C₁-C₄ haloalkoxy, or cyano;
one of R₄ and R₆ is H, halo, optionally substituted C₁-C₄ alkyl, optionally substituted C₁-C₄ alkoxy, optionally substituted C₁-C₄ haloalkyl, optionally substituted C₁-C₄ haloalkoxy, or cyano; and
the remaining R₄ or R₆ is: 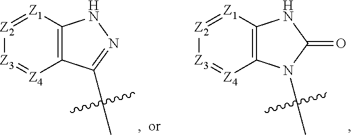
wherein
each of Z₁, Z₂, Z₃, and Z₄ is independently CR_{c} or N, wherein R_{c} is H, halo, optionally substituted C₁-C₄ alkyl, optionally substituted C₁-C₄ alkoxy, optionally substituted C₁-C₄ haloalkyl, optionally substituted C₁-C₄ haloalkoxy, or cyano.

2. The compound of claim 1, wherein R₄ or R₆ is

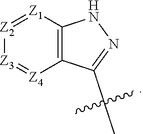

3. The compound of claim 2, wherein the compound is a compound of formula (I-2): 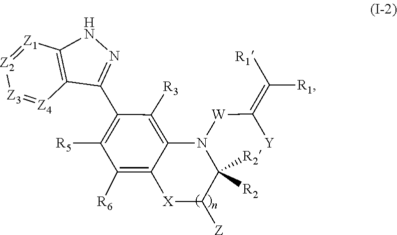
or a pharmaceutically acceptable salt thereof.

4. The compound of claim 2, wherein the compound is a compound of formula (I-3):

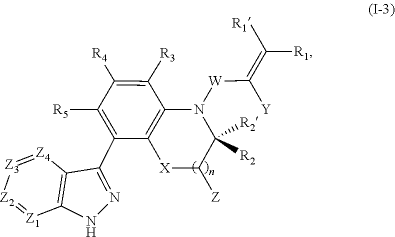

5. The compound of claim 1, wherein R₄ or R₆ is

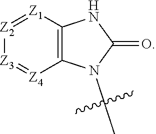

6. The compound of claim 5, wherein the compound has the structure of formula (I-4): 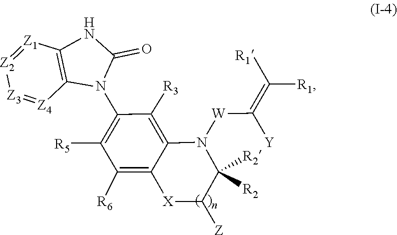
or a pharmaceutically acceptable salt thereof.

7. The compound of claim 5, wherein the compound has the structure of formula (I-5): 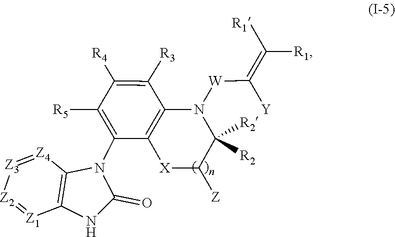
or a pharmaceutically acceptable salt thereof.

8. The compound of claim 3 or 6, wherein R₆ is H.

9. The compound of claim 4 or 7, wherein R₄ is H.

10. The compound of any one of claims 1-9, wherein Z₁ is CR_{c}.

11. The compound of claim 10, wherein Z₁ is CH.

12. The compound of claim 10, wherein Z₁ is CR_{c}, wherein R_{c} is optionally substituted C₁-C₄ alkyl.

13. The compound of claim 12, wherein Z₁ is CCH₃.

14. The compound of any one of claims 1-9, wherein Z₁ is CR_{c}, wherein R_{c} is cyano.

15. The compound of any one of claims 1-9, wherein Z₁ is N.

16. The compound of any one of claims 1-15, wherein Z₂ is CR_{c}.

17. The compound of claim 16, wherein Z₂ is CH.

18. The compound of claim 16, wherein Z₂ is CR_{c}, wherein R_{c} is optionally substituted C₁-C₄ alkyl.

19. The compound of claim 18, wherein Z₂ is CCH₃.

20. The compound of claim 16, wherein Z₂ is CR_{c}, and R_{c} is cyano.

21. The compound of any one of claims 1-15, wherein Z₂ is N.

22. The compound of any one of claims 1-21, wherein Z₃ is CR_{c}.

23. The compound of claim 22, wherein Z₃ is CH.

24. The compound of claim 22, wherein Z₃ is CR_{c}, wherein R_{c} is optionally substituted C₁-C₄ alkyl.

25. The compound of claim 24, wherein Z₃ is CCH₃.

26. The compound of claim 22, wherein Z₃ is CR_{c}, and R_{c} is cyano.

27. The compound of any one of claims 1-21, wherein Z₃ is N.

28. The compound of any one of claims 1-27, wherein Z₄ is CR_{c}.

29. The compound of claim 28, wherein Z₄ is CH.

30. The compound of claim 28, wherein Z₄ is CR_{c}, wherein R_{c} is optionally substituted C₁-C₄ alkyl.

31. The compound of claim 30, wherein Z₄ is CCH₃.

32. The compound of claim 28, wherein Z₄ is CR_{c}, and R_{c} is cyano.

33. The compound of any one of claims 1-27, wherein Z₄ is N.

34. The compound of any one of claims 1-33, wherein W is C(O).

35. The compound of any one of claims 1-33, wherein W is S(O)₂.

36. The compound of any one of claims 1-35, wherein R₁ is cyano.

37. The compound of any one of claims 1-35, wherein R₁ is CH₂N(Rₐ)₂.

38. The compound of claim 37, wherein each Rₐ is optionally substituted C₁-C₄ alkyl.

39. The compound of claim 38, wherein each Rₐ is methyl.

40. The compound of any one of claims 1-35, wherein R₁ is C(O)ORₐ′.

41. The compound of claim 40, wherein Rₐ′ is optionally substituted C₁-C₄ alkyl.

42. The compound of claim 41, wherein Rₐ′ is ethyl.

43. The compound of any one of claims 1-35, wherein R₁ is optionally substituted C₁-C₄ alkyl.

44. The compound of claim 43, wherein R₁ is methyl.

45. The compound of any one of claims 1-44, wherein Y is halo.

46. The compound of claim 45, wherein Y is fluoro.

47. The compound of any one of claims 1-44, wherein Y is H.

48. The compound of any one of claims 1-47, wherein R₂ is H.

49. The compound of any one of claims 1-47, wherein R₂ is optionally substituted C₁-C₄ alkyl.

50. The compound of claim 49, wherein R₂ is methyl.

51. The compound of any one of claims 1-47, wherein R₂ is optionally substituted C₃-C₆ cycloalkyl.

52. The compound of any one of claims 1-51, wherein R₂′ is H.

53. The compound of any one of claims 1-51, wherein R₂′ is optionally substituted C₁-C₄ alkyl.

54. The compound of claim 53, wherein R₂′ is methyl.

55. The compound of any one of claims 1-51, wherein R₂′ is optionally substituted C₃-C₆ cycloalkyl.

56. The compound of any one of claims 1-55, wherein n is 1.

57. The compound of any one of claims 1-55, wherein n is 0.

58. The compound of any one of claims 1-57, wherein X is C(R_{b})₂.

59. The compound of claim 58, wherein each R_{b} is H.

60. The compound of claim 58, wherein each R_{b} is optionally substituted C₁-C₄ alkyl.

61. The compound of any one of claims 1-57, wherein X is O.

62. The compound of any one of claims 1-58, wherein X is NR_{b}′.

63. The compound of claim 62, wherein R_{b}′ is H.

64. The compound of claim 62, wherein R_{b}′ is optionally substituted C₁-C₄ alkyl.

65. The compound of claim any one of claims 1-64, wherein R₃ is H.

66. The compound of any one of claims 1-65, wherein R₅ is H.

67. A compound of structure: 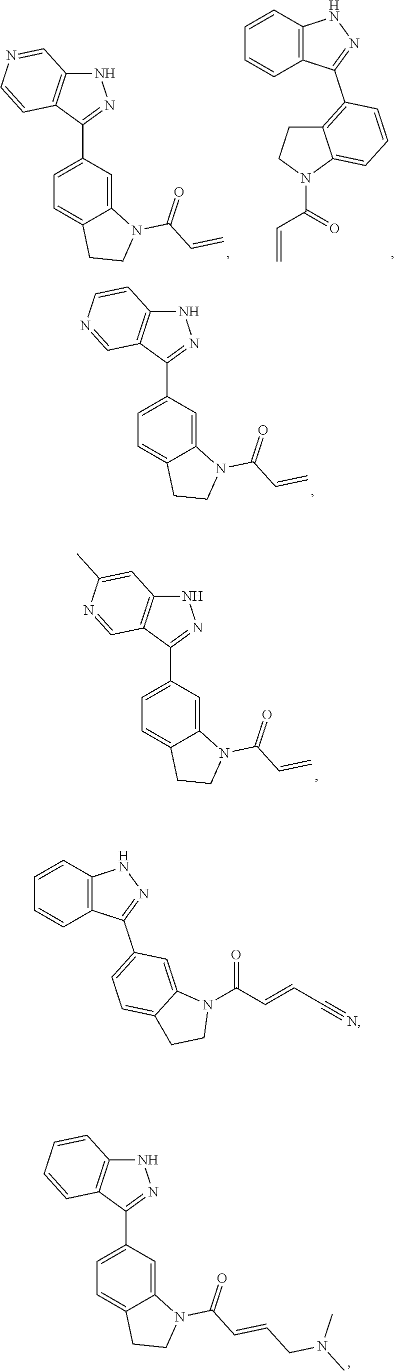 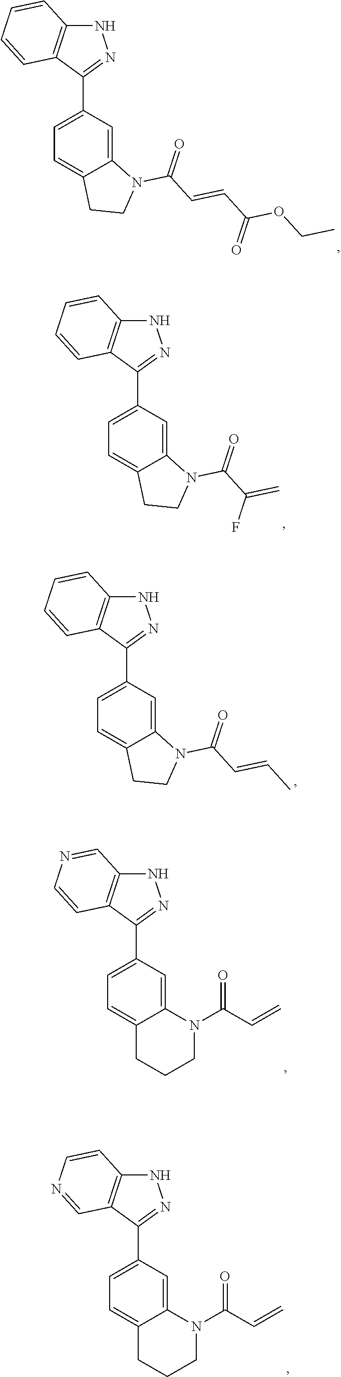 [SMILES: CCOC(=O)/C=C/C(=O)N1CCc2ccc(-c3n[nH]c4ccccc34)cc21 ; C=C(F)C(=O)N1CCc2ccc(-c3n[nH]c4ccccc34)cc21 ; C/C=C/C(=O)N1CCc2ccc(-c3n[nH]c4ccccc34)cc21 ; C=CC(=O)N1CCCc2ccc(-c3n[nH]c4cnccc34)cc21 ; C=CC(=O)N1CCCc2ccc(-c3n[nH]c4ccncc34)cc21] 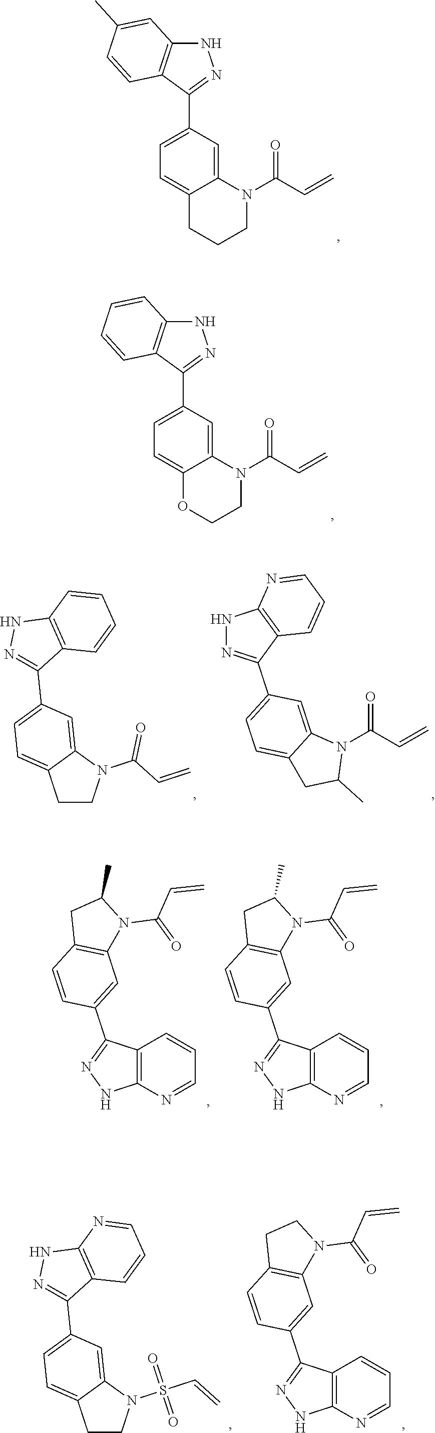 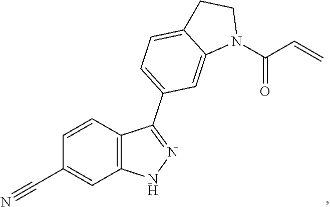 [SMILES: C=CC(=O)N1CCc2ccc(-c3n[nH]c4cc(C#N)ccc34)cc21]
or a pharmaceutically acceptable salt thereof.

68. A pharmaceutical composition comprising a compound of any one of claims 1-67 or a pharmaceutically acceptable salt thereof, and a pharmaceutically acceptable excipient.

69. A method for inhibiting an MKK7 enzyme, comprising administering to a subject in need thereof a therapeutically effective amount of a compound of any one of claims 1-67 or a pharmaceutically acceptable salt thereof.

70. A method for increasing survival or reducing death or degeneration of a damaged or degenerating neuron, the method comprising contacting the damaged or degenerating neuron by administering to a subject in need thereof a therapeutically effective amount of a compound of any one of claims 1-67 or a pharmaceutically acceptable salt thereof.

71. A method for treating an inflammatory disorder or cancer by administering to a subject in need thereof a therapeutically effective amount of a compound of any one of claims 1-67 or a pharmaceutically acceptable salt thereof.

72. The method of claim 71, wherein the inflammatory disorder or cancer is neuroblastoma, glioblastoma, or intraocular inflammation.

73. A method for treating a disorder associated with MKK7-related pathway regulation, comprising administering to a subject in need thereof a therapeutically effective amount of a compound of any one of claims 1-67 or a pharmaceutically acceptable salt thereof.

74. The method of claim 73, wherein the disorder is associated with an MKK7 enzyme.

75. The method of claim 73 or 74, wherein the disorder is a neurodegenerative disease, a neurotraumatic disorder, a neurodevelopmental disorder, or an affective disorder.

76. The method of claim 73 or 74, wherein the disorder is amyotrophic lateral sclerosis (ALS), glaucoma, chemotherapy-induced peripheral neuropathy, Alzheimer's disease, Parkinson's disease, Huntington's disease, multiple sclerosis (MS), optic neuropathy, neuroblastoma, glioblastoma, lysosomal storage disorders, traumatic brain injury, spinal cord injury, spinal cord crush, optic nerve injury, or a combination thereof.
